(19) United States
(12) Patent Application Publication
(10) Pub. No.: US 2022/0289680 A1
(43) Pub. Date: Sep. 15, 2022

(54) METHODS FOR MAKING DIMERIC NAPHTHALIMIDES AND SOLID STATE FORMS OF THE SAME

(71) Applicant: Alucent Biomedical, Inc., Salt Lake City, UT (US)

(72) Inventors: Kevin S. WARNER, Salt Lake City, UT (US); Denis Viktorovich AREFYEV, Florence, SC (US); Michael P. CRUSKIE, Florence, SC (US); Chaminda Priyapushpa GAMAGE, Florence, SC (US); Katherine Michelle THOMAS, Lamar, SC (US)

(21) Appl. No.: 17/418,296

(22) PCT Filed: Dec. 26, 2019
(86) PCT No.: PCT/US19/68611

§ 371 Date: Jun. 25, 2021

Related U.S. Application Data
(60) Provisional application No. 62/785,477, filed on Dec. 27, 2018.

(51) Int. Cl. C07D 221/14

(52) U.S. Cl. CPC C07D 221/14; C07B 2200/13

(57) ABSTRACT

The present disclosure provides methods of preparing Compound of Formula (I), solid state forms of the same and compositions comprising the same. Also disclosed herein are methods of preparing a diacetate salt of Compound of Formula (I) and pharmaceutical compositions comprising the same.

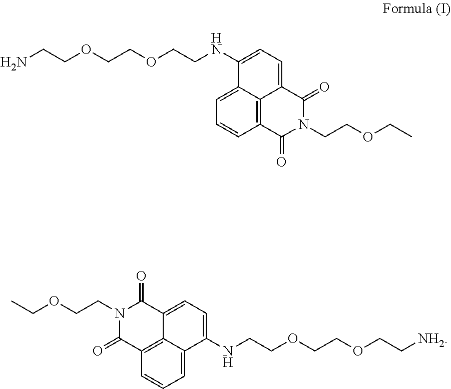

The present disclosure provides novel methods for making a dimeric naphthalimide, compositions comprising the same, solid state forms of the same, methods for using the same, methods for improving the synthetic yield of the dimeric naphthalimide, and methods of reducing impurities in a composition comprising the dimeric naphthalimide.

Certain dimeric naphthalimide compounds have been previously disclosed. See, e.g., U.S. Pat. No. 6,410,505 B2. For example, a dimeric naphthalimide compound, 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione), also known as 10-8-10 dimer, 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbix(oxy-2,1-ethanediyl)]bis[6-((2-[2-(2-aminoethoxy)ethoxy]ethyl)amino)-1H-benzo[de]isoquinoline-1,3(2H)-dione]; and 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9Cl), and herein referred to as Compound of Formula (I), had been disclosed. Id.

The present disclosure provides solid state forms (i.e., Form 1, Form 2, and non-crystalline) of the Compound of Formula (I):

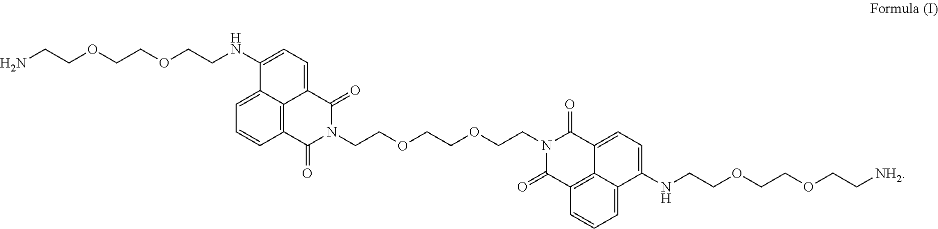 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

It was surprisingly and unpredictably discovered that the Compound of Formula (I) exists in multiple solid state forms. It was also surprisingly and unpredictably discovered that methods for making Compound of Formula (I) comprising at least one crystallization and/or at least one purification other than crystallization as disclosed herein may result in a Compound of Formula (I) of improved purity relative to a Compound of Formula (I) prepared by a synthetic method that does not comprise at least one crystallization and/or at least one purification other than crystallization.

In some embodiments, provided herein is a method for making Form 1 of 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione) (“Compound of Formula (I)”) comprising isolating Form 1 from a mixture comprising Compound of Formula (I) and at least one solvent.

In some embodiments, provided herein is a method for making a Compound of Formula (I) comprising slurrying Compound of Formula (I) in a solvent mixture comprising at least one first solvent chosen from acetonitrile, isopropanol, acetone, ethanol, and tetrahydrofuran; and water.

In some embodiments, provided herein is a method of improving the synthetic yield of Compound of Formula (I) comprising slurrying Compound of Formula (I) in a solvent mixture comprising a first solvent chosen from acetonitrile, isopropanol, acetone, ethanol, and tetrahydrofuran; and water.

In some embodiments, provided herein is a method of reducing process impurities in a composition comprising Compound of Formula (I), comprising slurrying Compound of Formula (I) in a solvent mixture comprising at least one first solvent chosen from acetonitrile, ethanol, methylethylketone, and tetrahydrofuran; and water.

In some embodiments, provided herein is a method for making a diacetate salt of Compound of Formula (I) comprising isolating the diacetate salt of Compound of Formula (I) from a mixture comprising the diacetate salt of Compound of Formula (I), activated carbon, and at least one solvent.

In some embodiments, provided herein is a pharmaceutical composition comprising a diacetate salt of Compound of Formula (I) formed from combining the diacetate salt of Compound of Formula (I) produced according to a method described herein or a diacetate salt of Compound of Formula (I) as described herein and at least one additional component chosen from pharmaceutically acceptable carriers, pharmaceutically acceptable vehicles, and pharmaceutically acceptable excipients.

In some embodiments, provided herein is Form 2 of Compound of Formula (I).

In some embodiments, provided herein is a method for making Form 2 of Compound of Formula (I) comprising isolating Form 2 from a mixture comprising Compound of Formula (I) and at least one first solvent.

In some embodiments, provided herein is a method for making Form 2 of Compound of Formula (I) comprising slurrying Compound of Formula (I) in a solvent mixture comprising acetone and water.

In some embodiments, provided herein is a pharmaceutical composition comprising Compound of Formula (I) formed from combining Form 2 of the Compound of Formula (I) as described herein and at least one additional component chosen from pharmaceutically acceptable carriers, pharmaceutically acceptable vehicles, and pharmaceutically acceptable excipients.

### BRIEF DESCRIPTION OF DRAWINGS

FIG. 1 shows an X-ray powder diffraction (XRPD) pattern of a non-crystalline solid sample of a Compound of Formula (I) isolated from a mixture of Compound of Formula (I), 1,1,1,3,3,3-hexafluoro-2-propanol (solvent), and acetone (anti-solvent) after being cooled from 60° C. to −15° C. over 5 days.

FIG. 2 shows an XRPD pattern of Form 1 of a Compound of Formula (I) isolated from a mixture of a Compound of Formula (I), 1,1,1,3,3,3-hexafluoro-2-propanol (solvent), and acetonitrile (anti-solvent) after being cooled from 60° C. to −15° C. over 5 days.

FIG. 3 shows an XRPD pattern of a non-crystalline solid sample of a Compound of Formula (I) isolated from a mixture of a Compound of Formula (I), 1,1,1,3,3,3-hexafluoro-2-propanol (solvent), and 1,4-dioxane (anti-solvent) after being cooled from 60° C. to −15° C. over 5 days.

FIG. 4 shows an XRPD pattern of Form 1 of Compound of Formula (I) isolated from a mixture of a Compound of Formula (I), 1,1,1,3,3,3-hexafluoro-2-propanol (solvent), and ethanol (anti-solvent) after being cooled from 60° C. to −15° C. over 5 days.

FIG. 5 shows an XRPD pattern of a non-crystalline solid sample of a Compound of Formula (I) isolated from a mixture of a Compound of Formula (I), 1,1,1,3,3,3-hexafluoro-2-propanol (solvent) and ethyl acetate (anti-solvent) after being cooled from 60° C. to −15° C. over 5 days.

FIG. 6 shows an XRPD pattern of Form 1 of a Compound of Formula (I) isolated from a mixture of a Compound of Formula (I), 1,1,1,3,3,3-hexafluoro-2-propanol (solvent) and methanol (anti-solvent) after being cooled from 60° C. to −15° C. over 5 days.

FIG. 7 shows an XRPD pattern of Form 1 of a Compound of Formula (I) isolated from a mixture of a Compound of Formula (I), 1,1,1,3,3,3-hexafluoro-2-propanol (solvent) and methyl ethyl ketone (anti-solvent) after being cooled from 60° C. to −15° C. over 5 days.

FIG. 8 shows an XRPD pattern of a non-crystalline sample of a Compound of Formula (I) isolated from a mixture of a Compound of Formula (I), 1,1,1,3,3,3-hexafluoro-2-propanol (solvent) and 2-methyl-tetrahydrofuran (anti-solvent) after being cooled from 60° C. to −15° C. over 5 days.

FIG. 9 shows an XRPD pattern of a non-crystalline solid sample of a Compound of Formula (I) isolated from a mixture of a Compound of Formula (I), 1,1,1,3,3,3-hexafluoro-2-propanol (solvent) and 2-propanol (anti-solvent) after being cooled from 60° C. to −15° C. over 5 days.

FIG. 10 shows an XRPD pattern of a non-crystalline solid sample of a Compound of Formula (I) isolated from a mixture of a Compound of Formula (I), 1,1,1,3,3,3-hexafluoro-2-propanol (solvent) and tetrahydrofuran (anti-solvent) after being cooled from 60° C. to −15° C. over 5 days.

FIG. 11 shows an XRPD pattern of Form 1 of a Compound of Formula (I) isolated from a mixture of a Compound of Formula (I), 1,1,1,3,3,3-hexafluoro-2-propanol (solvent) and toluene (anti-solvent) after being cooled from 60° C. to −15° C. over 5 days.

FIG. 12 shows an XRPD pattern of a non-crystalline solid sample of a Compound of Formula (I) isolated from a mixture of a Compound of Formula (I), 1,1,1,3,3,3-hexafluoro-2-propanol (solvent) and water (anti-solvent) after being cooled from 60° C. to −15° C. over 5 days.

FIG. 13 shows an XRPD pattern of Form 1 of a Compound of Formula (I) isolated from a mixture of a Compound of Formula (I), 2,2,2-trifluoroethanol (solvent), and acetone (anti-solvent) after being cooled from 60° C. to −15° C. over 5 days.

FIG. 14 shows an XRPD pattern of Form 1 of a Compound of Formula (I) isolated from a mixture of a Compound of Formula (I), 2,2,2-trifluoroethanol (solvent), and acetonitrile (anti-solvent) after being cooled from 60° C. to −15° C. over 5 days.

FIG. 15 shows an XRPD pattern of a non-crystalline solid sample of a Compound of Formula (I) isolated from a mixture of a Compound of Formula (I), 2,2,2-trifluoroethanol (solvent), and 1,4-dioxane (anti-solvent) after being cooled from 60° C. to −15° C. over 5 days.

FIG. 16 shows an XRPD pattern of Form 1 of a Compound of Formula (I) isolated from a mixture of a Compound of Formula (I), 2,2,2-trifluoroethanol (solvent), and ethanol (anti-solvent) after being cooled from 60° C. to −15° C. over 5 days.

FIG. 17 shows an XRPD pattern of Form 1 of a Compound of Formula (I) isolated from a mixture of a Compound of Formula (I), 2,2,2-trifluoroethanol (solvent), and ethyl acetate (anti-solvent) after being cooled from 60° C. to −15° C. over 5 days.

FIG. 18 shows an XRPD pattern of Form 1 of a Compound of Formula (I) isolated from a mixture of a Compound of Formula (I), 2,2,2-trifluoroethanol (solvent), and methanol (anti-solvent) after being cooled from 60° C. to −15° C. over 5 days.

FIG. 19 shows an XRPD pattern of Form 1 of a Compound of Formula (I) isolated from a mixture of a Compound of Formula (I), 2,2,2-trifluoroethanol (solvent), and methyl ethyl ketone (anti-solvent) after being cooled from 60° C. to −15° C. over 5 days.

FIG. 20 shows an XRPD pattern of Form 1 of a Compound of Formula (I) isolated from a mixture of a Compound of Formula (I), 2,2,2-trifluoroethanol (solvent), and 2-methyl-tetrahydrofuran (anti-solvent) after being cooled from 60° C. to −15° C. over 5 days.

FIG. 21 shows an XRPD pattern of Form 1 of a Compound of Formula (I) isolated from a mixture of a Compound of Formula (I), 2,2,2-trifluoroethanol (solvent), and 2-propanol (anti-solvent) after being cooled from 60° C. to −15° C. over 5 days.

FIG. 22 shows an XRPD pattern of Form 1 of a Compound of Formula (I) isolated from a mixture of a Compound of Formula (I), 2,2,2-trifluoroethanol (solvent), and tetrahydrofuran (anti-solvent) after being cooled from 60° C. to −15° C. over 5 days.

FIG. 23 shows an XRPD pattern of Form 1 of a Compound of Formula (I) isolated from a mixture of a Compound of Formula (I), 2,2,2-trifluoroethanol (solvent), and toluene (anti-solvent) after being cooled from 60° C. to −15° C. over 5 days.

FIG. 24 shows an XRPD pattern of a non-crystalline solid sample of a Compound of Formula (I) isolated from a mixture of a Compound of Formula (I), 2,2,2-trifluoroethanol (solvent), and water (anti-solvent) after being cooled from 60° C. to −15° C. over 5 days.

FIG. 25 shows an XRPD pattern of Form 1 of a Compound of Formula (I) isolated from a mixture of a Compound of Formula (I) and 1,1,1,1,3,3,3-hexafluoro-2-propanol after evaporation of the solvent at room temperature.

FIG. 26 shows an XRPD pattern of Form 1 of a Compound of Formula (I) isolated from a mixture of a Compound of Formula (I) and 2,2,2-trifluoroethanol after evaporation of the solvent at room temperature.

FIG. 27 shows an XRPD pattern of a non-crystalline solid of a Compound of Formula (I) isolated from a slurry of a Compound of Formula (I) in acetone at room temperature after seven days.

FIG. 28 shows an XRPD pattern of Form 1 of a Compound of Formula (I) isolated from a slurry of a Compound of Formula (I) in acetone at 55° C. after seven days.

FIG. 29 shows an XRPD pattern of Form 1 of a Compound of Formula (I) isolated from a slurry of a Compound of Formula (I) in acetonitrile at room temperature after seven days.

FIG. 30 shows an XRPD pattern of Form 1 of a Compound of Formula (I) isolated from a slurry of a Compound of Formula (I) in acetonitrile at 55° C. after seven days.

FIG. 31 shows an XRPD pattern of Form 1 of a Compound of Formula (I) isolated from a slurry of a Compound of Formula (I) in chloroform at room temperature after seven days.

FIG. 32 shows an XRPD pattern of Form 1 of a Compound of Formula (I) isolated from a slurry of a Compound of Formula (I) in dichloromethane at room temperature after seven days.

FIG. 33 shows an XRPD pattern of Form 1 of a Compound of Formula (I) isolated from a slurry of a Compound of Formula (I) in 1,4-dioxane at room temperature after seven days.

FIG. 34 shows an XRPD pattern of Form 1 of a Compound of Formula (I) isolated from a slurry of a Compound of Formula (I) in N,N-dimethylformamide at room temperature after seven days.

FIG. 35 shows an XRPD pattern of Form 1 of a Compound of Formula (I) isolated from a slurry of a Compound of Formula (I) in N,N-dimethylformamide at 55° C. after seven days.

FIG. 36 shows an XRPD pattern of Form 1 of a Compound of Formula (I) isolated from a slurry of a Compound of Formula (I) in dimethylsulfoxide at room temperature after seven days.

FIG. 37 shows an XRPD pattern of Form 1 of a Compound of Formula (I) isolated from a slurry of a Compound of Formula (I) in ethanol at room temperature after seven days.

FIG. 38 shows an XRPD pattern of Form 1 of a Compound of Formula (I) isolated from a slurry of a Compound of Formula (I) in ethanol at 55° C. after seven days.

FIG. 39 shows an XRPD pattern of Form 1 of a Compound of Formula (I) isolated from a slurry of a Compound of Formula (I) in ethyl acetate at room temperature after seven days.

FIG. 40 shows an XRPD pattern of Form 1 of a Compound of Formula (I) isolated from a slurry of a Compound of Formula (I) in ethyl acetate at 55° C. after seven days.

FIG. 41 shows an XRPD pattern of Form 1 of a Compound of Formula (I) isolated from a slurry of a Compound of Formula (I) in diethyl ether at room temperature after seven days.

FIG. 42 shows an XRPD pattern of Form 1 of a Compound of Formula (I) isolated from a slurry of a Compound of Formula (I) in diethyl ether at 40° C. after seven days.

FIG. 43 shows an XRPD pattern of Form 1 of a Compound of Formula (I) isolated from a slurry of a Compound of Formula (I) in methanol at room temperature after seven days.

FIG. 44 shows an XRPD pattern of Form 1 of a Compound of Formula (I) isolated from a slurry of a Compound of Formula (I) in methanol at 55° C. after seven days.

FIG. 45 shows an XRPD pattern of Form 1 of a Compound of Formula (I) isolated from a slurry of a Compound of Formula (I) in methyl ethyl ketone at room temperature after seven days.

FIG. 46 shows an XRPD pattern of Form 1 of a Compound of Formula (I) isolated from a slurry of a Compound of Formula (I) in methyl ethyl ketone at 55° C. after seven days.

FIG. 47 shows an XRPD pattern of Form 1 of a Compound of Formula (I) isolated from a slurry of a Compound of Formula (I) in 2-methyl-tetrahydrofuran at room temperature after seven days.

FIG. 48 shows an XRPD pattern of Form 1 of a Compound of Formula (I) isolated from a slurry of a Compound of Formula (I) in 2-propanol at room temperature after seven days.

FIG. 49 shows an XRPD pattern of Form 1 of a Compound of Formula (I) isolated from a slurry of a Compound of Formula (I) in tetrahydrofuran at room temperature after seven days.

FIG. 50 shows an XRPD pattern of Form 1 of a Compound of Formula (I) isolated from a slurry of a Compound of Formula (I) in tetrahydrofuran at 55° C. after seven days.

FIG. 51 shows an XRPD pattern of Form 1 of a Compound of Formula (I) isolated from a slurry of a Compound of Formula (I) in toluene at room temperature after seven days.

FIG. 52 shows an XRPD pattern of Form 1 of a Compound of Formula (I) isolated from a slurry of a Compound of Formula (I) in water at room temperature after seven days.

FIG. 53 shows an XRPD pattern of Form 1 of a Compound of Formula (I) isolated from a slurry of a Compound of Formula (I) in water at 55° C. after seven days.

FIG. 54 shows an XRPD pattern of Form 2 of a Compound of Formula (I) isolated from a slurry of a Compound of Formula (I) in acetone/water (95/5, v/v) at room temperature after seven days.

FIG. 55 shows an XRPD pattern of Form 2 of a Compound of Formula (I) isolated from a slurry of a Compound of Formula (I) in acetone/water (95/5, v/v) at 55° C. after seven days.

FIG. 56 shows an XRPD pattern of Form 1 of a Compound of Formula (I) isolated from a slurry of a Compound of Formula (I) in acetonitrile/water (95/5, v/v) at room temperature after seven days.

FIG. 57 shows an XRPD pattern of Form 1 of a Compound of Formula (I) isolated from a slurry of a Compound of Formula (I) in ethanol/water (95/5, v/v) at room temperature after seven days.

FIG. 58 shows an XRPD pattern of Form 1 of a Compound of Formula (I) isolated from a slurry of a Compound of Formula (I) in tetrahydrofuran/water (95/5, v/v) at room temperature after seven days.

FIG. 59 shows an XRPD pattern of Form 1 of a Compound of Formula (I) isolated after heating a sample of a Compound of Formula (I) at 145° C. for one minute.

FIG. 60 shows an overlay of XRPD patterns of samples of Compound of Formula (I) obtained from: an acetone/water slurry (trace A); an acetonitrile slurry (trace B); ether slurry (trace C); 2,2,2-trifluoroethanol evaporation (trace D); and synthetic method similar to the method of Example 4 (trace E).

FIG. 61 shows a dynamic scanning calorimetry (DSC) trace of Form 1 of a Compound of Formula (I), which shows endotherms at 139.1° C. and 191.6° C.

FIG. 62 shows a dynamic vapor sorption (DVS) trace of Form 1 of a Compound of Formula (I), which shows 0.6% weight loss upon drying at 5% relative humidity; 13.04% weight gain when cycling from 5% to 95% relative humidity; and 13.12% weight loss when cycling from 95% to 5% relative humidity.

FIG. 63 shows an XRPD pattern of Form 1 of a Compound of Formula (I) after the sample was subjected to DVS analysis.

FIG. 64 shows an upfield expansion of a solution phase proton nuclear magnetic resonance (¹H-NMR) spectrum of Form 1 of a Compound of Formula (I) dissolved in hexa-deuterated dimethylsulfoxide (DMSO-d₆).

FIG. 65 shows a downfield expansion of a solution phase ¹H-NMR spectrum of Form 1 of a Compound of Formula (I) dissolved in DMSO-d₆.

FIG. 66 shows a DSC trace and a thermogravimetric analysis (TGA) trace of a solid sample of Form 2 of a Compound of Formula (I). The DSC trace shows endotherms at 75.4° C. and 181.3° C.; the TGA trace shows a loss of approximately 9.2% sample weight when heating from room temperature to 100° C.

FIG. 67 shows a DVS trace of a solid sample of Form 2 of a Compound of Formula (I), which shows 1.44% weight loss upon drying at 5% relative humidity; 7.2% weight gain when cycling from 5% to 95% relative humidity; and 14.63% weight loss when cycling from 95% to 5% relative humidity.

FIG. 68 shows an XRPD pattern of a sample of Form 2 of a Compound of Formula (I) after a sample of Form 2 of a Compound of Formula (I) was subjected to DVS analysis.

FIG. 69 shows an XRPD pattern of a sample of Form 2 of a Compound of Formula (I) after a sample of Form 2 of a Compound of Formula (I) was heated at 80° C. for 20 minutes.

FIG. 70 shows an upfield expansion of a solution phase ¹H-NMR spectrum of a sample of Form 2 of a Compound of Formula (I) dissolved in DMSO-de.

FIG. 71 shows a downfield expansion of a solution phase ¹H-NMR spectrum of a sample of Form 2 of a Compound of Formula (I) dissolved in DMSO-d₆.

FIG. 72 shows an XRPD pattern of a sample of Form 1 of a Compound of Formula (I) synthesized by a method similar to the method of Example 4.

FIG. 73 shows a DSC trace and a TGA trace of Form 1 of a Compound of Formula (I) synthesized according to Example 3. The DSC trace shows endotherms at 140.8° C. and 192.0° C.; the TGA trace shows a loss of approximately 1.2% sample weight when heating from room temperature to 180° C.

FIG. 74 shows a DVS trace of a solid sample of Form 1 of a Compound of Formula (I) synthesized according to Example 3, which shows 0.66% weight loss upon drying at 5% relative humidity; 16.36% weight gain when cycling from 5% to 95% relative humidity; and 15.51% weight loss when cycling from 95% to 5% relative humidity.

FIG. 75 shows an XRPD pattern of a sample of Form 1 of a Compound of Formula (I) synthesized according to Example 3 after a sample of Form 1 of a Compound of Formula (I) was subjected to DVS analysis.

FIGS. 76A and 76B show optical microscopic depictions of a sample of Form 1 of a Compound of Formula (I) synthesized according Example 3 at 10× magnification.

FIGS. 77A and 77B show optical microscopic depictions of a sample of Form 1 of a Compound of Formula (I) synthesized according to Example 3 at 10× magnification.

FIG. 78 shows an upfield expansion of a solution phase ¹H-NMR spectrum of a sample of Form 1 of a Compound of Formula (I) synthesized according to Example 3, dissolved in DMSO-d₆.

FIG. 79 shows a downfield expansion of a solution phase ¹H-NMR spectrum of a sample of Form 1 of a Compound of Formula (I) synthesized according to Example 3, dissolved in DMSO-d₆.

FIG. 80 shows a solution phase ¹H-NMR spectrum of a sample of a Compound of Formula (I) synthesized according to Example 4.

FIG. 81 shows a solution phase ¹H-NMR spectrum of a sample of a diacetate salt of a Compound of Formula (I).

As used herein, the following definitions shall apply unless otherwise indicated.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “Compound of Formula (I)” includes one or more of the conformational forms of the compound. Unless stated otherwise, compounds depicted herein coexisting with tautomeric forms are within the scope of the disclosure. Additionally, unless stated otherwise, structures depicted herein are also meant to include compounds that differ only in the presence of one or more isotopically enriched atoms. For example, compounds having the depicted structures except for the replacement of hydrogen by deuterium or tritium, or the replacement of a carbon atom by ¹³C- or ¹⁴C-enriched carbon atom are within the scope of this disclosure.

The Compound of Formula (I) may be described by the structure:

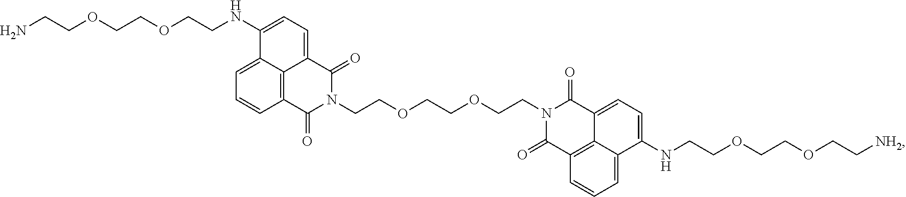 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

by the chemical names 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione); 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbix(oxy-2,1-ethanediyl)]bis[6-({2-[2-(2-aminoethoxy)ethoxy]ethyl}amino)-1H-benzo[de]isoquinoline-1,3(2H)-dione]; or 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9Cl), or by the Chemical Abstract Services (CAS) Registry No. 438200-66-9.

“Solid state form” as used herein encompasses non-crystalline, low-crystalline, and crystalline forms. In some embodiments, the solid state form of the Compound of Formula (I) is Form 1. In some embodiments, the solid state form of the Compound of Formula (I) is Form 2. In some embodiments, the solid state form of the Compound of Formula (I) is non-crystalline. The solid state forms can be identified and distinguished from each other by one or more analytical tests and/or physical properties such as, for example, X-ray powder diffraction (XRPD) diffractograms, single crystal structure, heat flow information from differential scanning calorimetry (DSC), absorption-desorption plots from dynamic vapor sorption (DVS), and/or thermodynamic stability. One of ordinary skill in the art will understand, however, that results from such analytical techniques may vary due to experimental error, such as by ±10%. For example, there may be variation in the intensities and/or peak positions in XRPD diffractograms even for the same crystalline form. Thus, those of ordinary skill in the art will understand that the signal maximum values in XRPD diffractograms (in degrees two-theta) referred to herein generally mean that value reported ±0.2 degrees two-theta of the reported value, an art-recognized variance.

A “signal” as used herein refers to a point in the XRPD pattern where the intensity, as measured in counts, is at a local maximum. One of ordinary skill in the art would recognize that one or more signals in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

As used herein, “a signal at . . . degrees two-theta,” “a signal at [a] two-theta value[ ] of . . . ” and/or “a signal at at least . . . two-theta value(s) chosen from . . . ” refer to X-ray reflection positions as measured and observed in X-ray powder diffraction experiments (degrees two-theta).

As used herein, the term “solvate” refers to a solid state form comprising one or more molecules of a compound of the present disclosure and, incorporated into the crystal lattice, one or more molecules of a solvent or solvents in stoichiometric or nonstoichiometric amounts.” Form 2 of the Compound of Formula (I) is an acetone solvate.

“Process impurities”, as used herein, refers to undesired chemical entities resulting form, e.g., undesired reaction pathways. In some embodiments, the presence and amount of process impurities is determined by LC.

“Isolating” or “isolation” as herein refer to separating a first component (e.g., Form 1) from other components. These terms encompass partial separation, i.e., where the first component is separated from some, but not all, of the other components. In other words, the concentration of the first component relative to other components increases when partially separated.

As used herein, an “increased synthetic yield” refers to a higher yield of a desired compound compared to, e.g., the synthetic yield of the same compound in a different reaction. Yield for a particular product of a reaction sequence can be determined by dividing the amount of the material obtained by the theoretical yield of the particular product.

“Pharmaceutically acceptable” as used herein to modify a carrier, vehicle, and/or excipient, refers to a nontoxic carrier, vehicle, and/or excipient, respectively, that does not destroy the pharmacological activity of the compound with which it is formulated.

As used herein, an X-ray powder diffractogram is “substantially as shown” in one or more of the figures herein when it is the same as that in the figure(s) taking into account possible variations in peak positions due to experimental variances and also due to measurement conditions employed, but not taking into account the magnitude (quantitative or relative) intensity of the peaks.

As used herein, the term “LC” means liquid chromatography and includes “HPLC” and “UPLC”, which refer to high performance liquid chromatography and ultra performance liquid chromatography, respectively.

Form 1 of Compound of Formula (I)

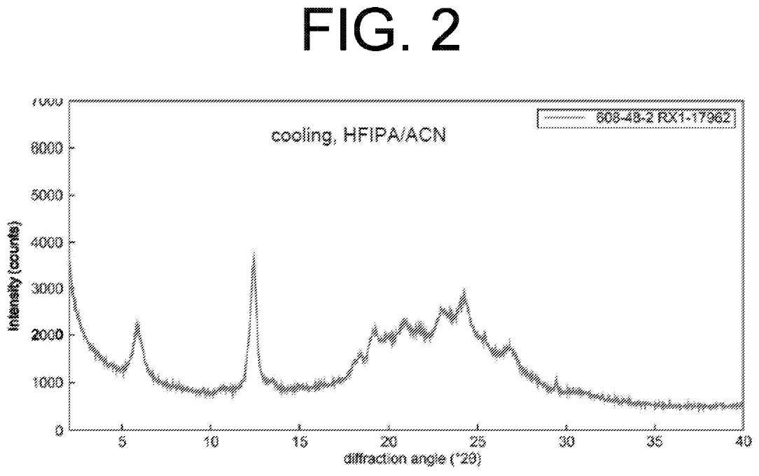

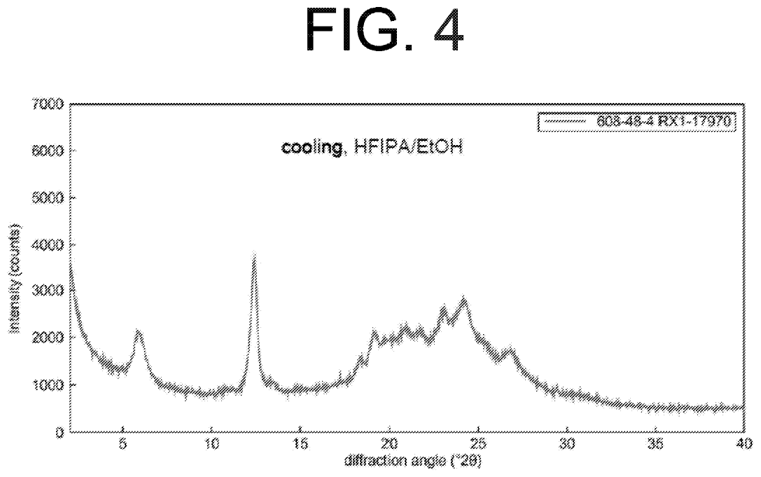

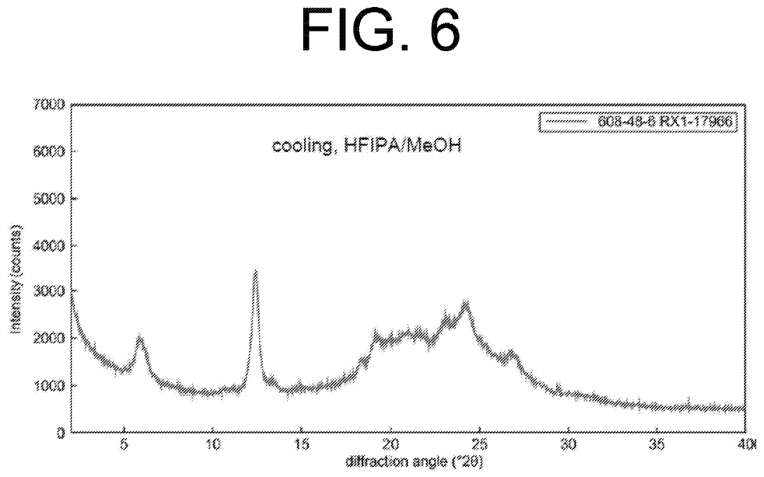

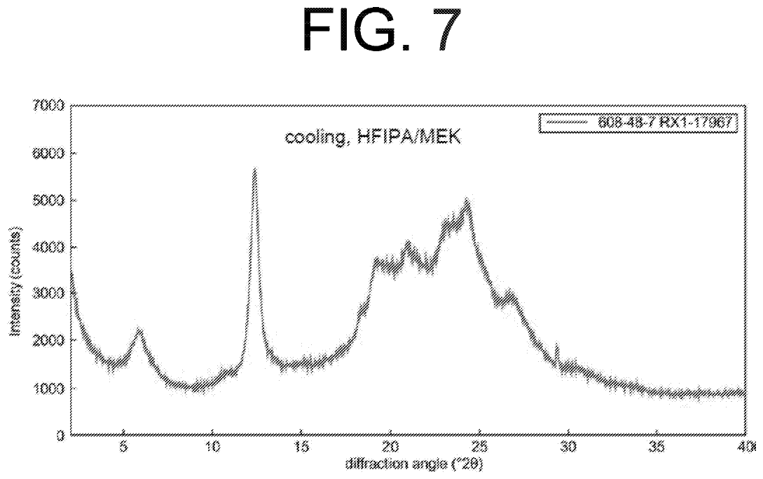

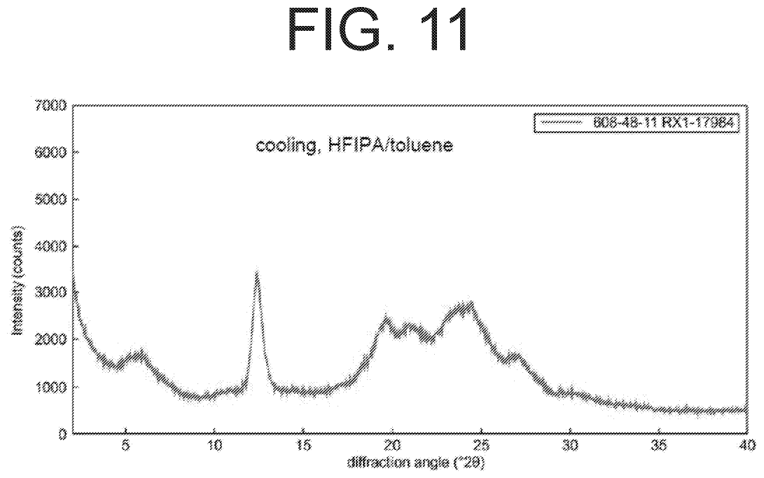

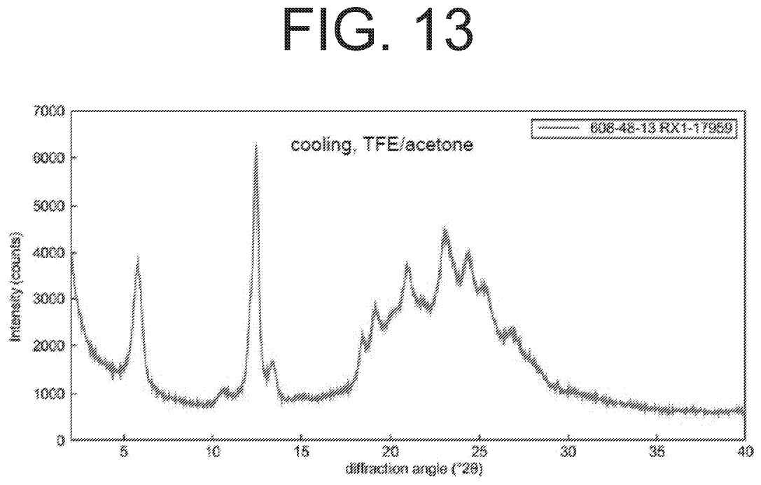

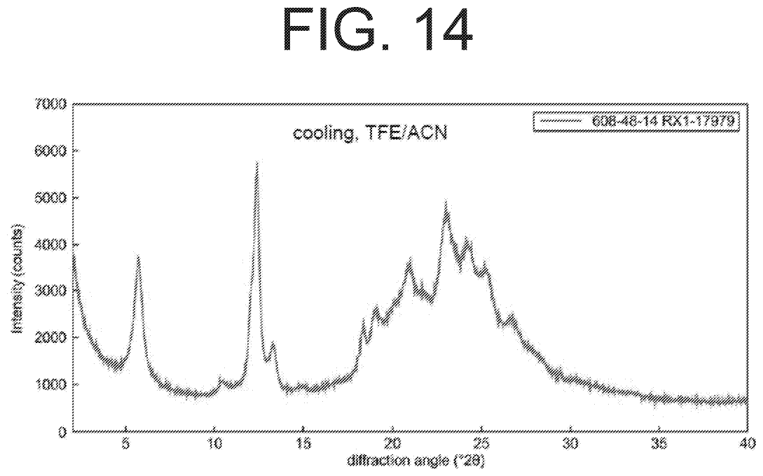

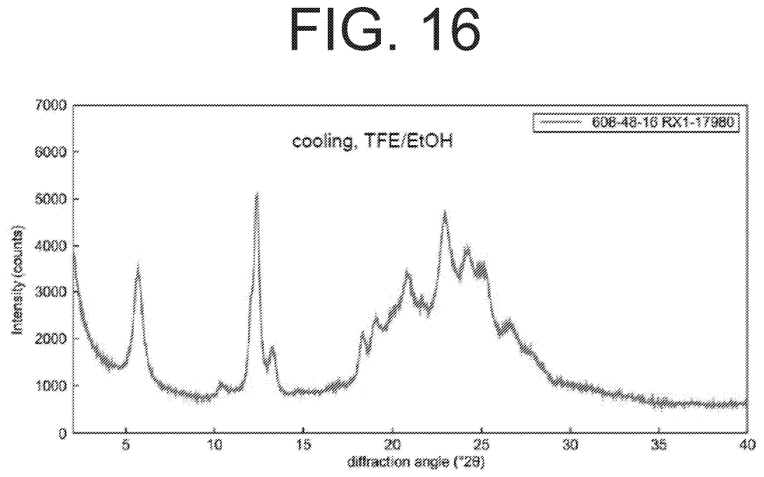

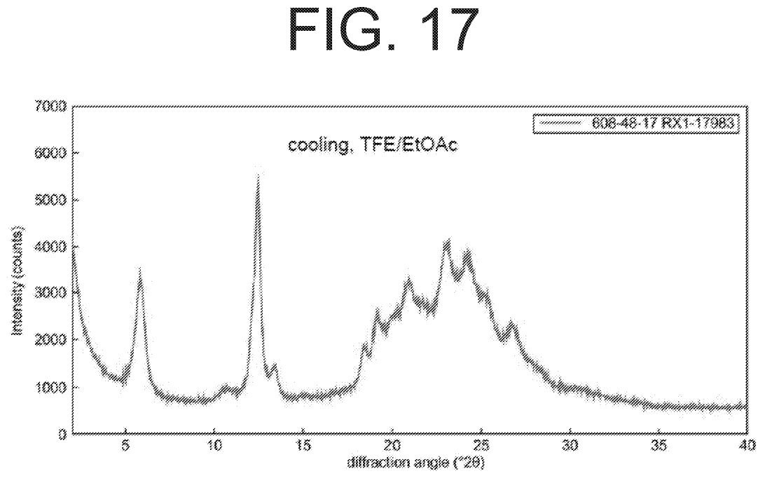

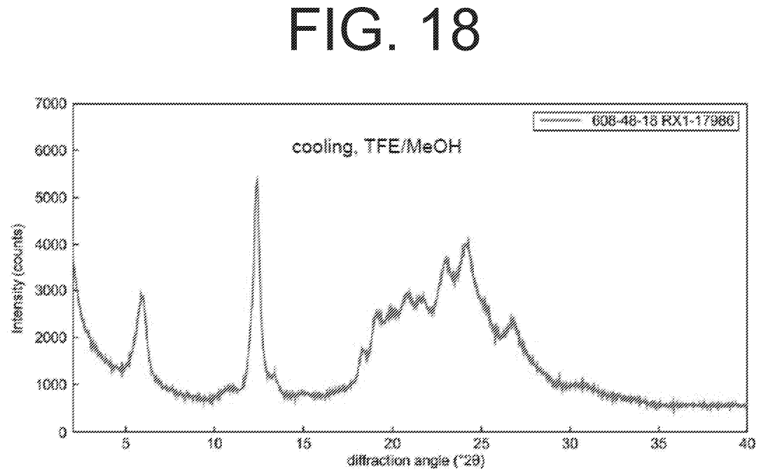

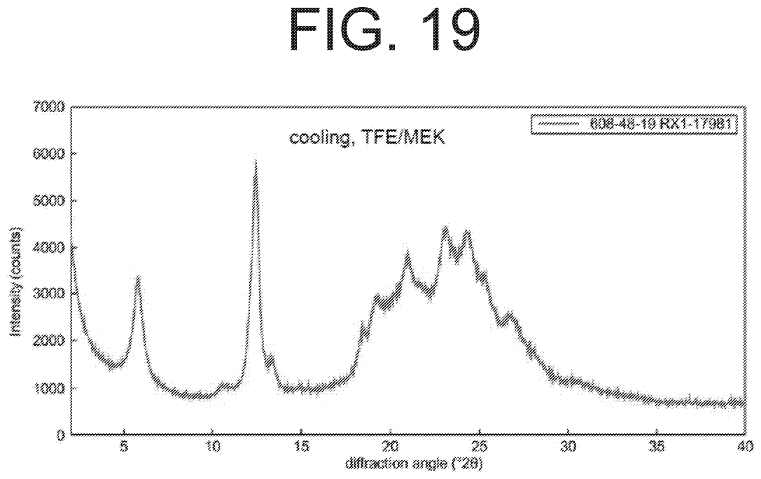

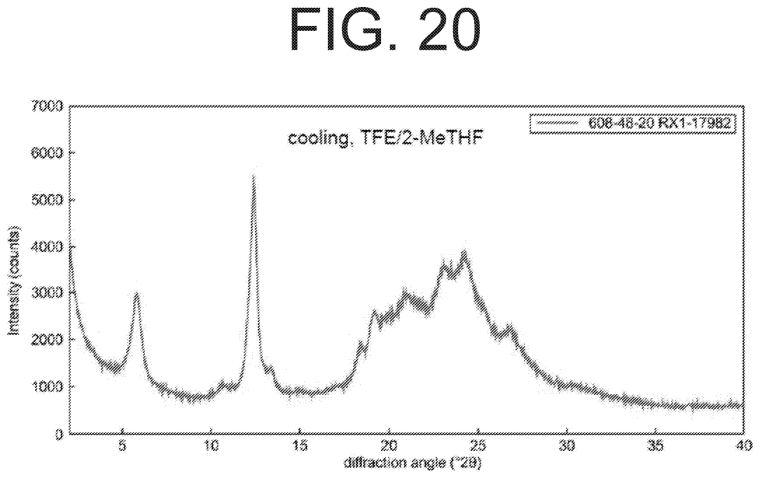

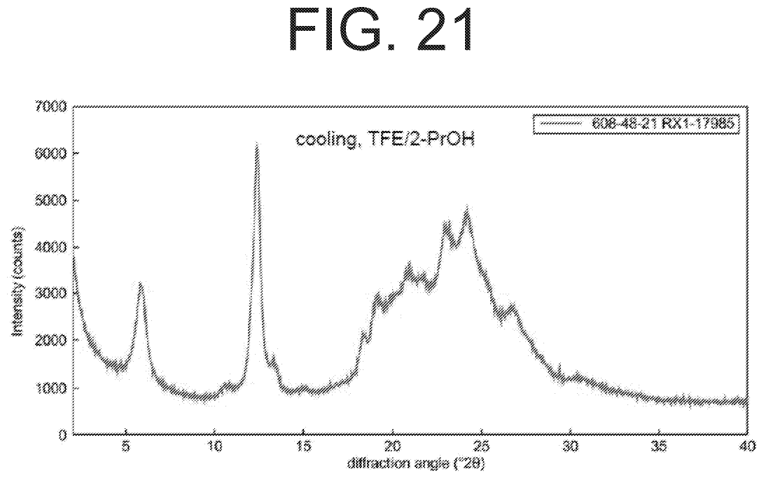

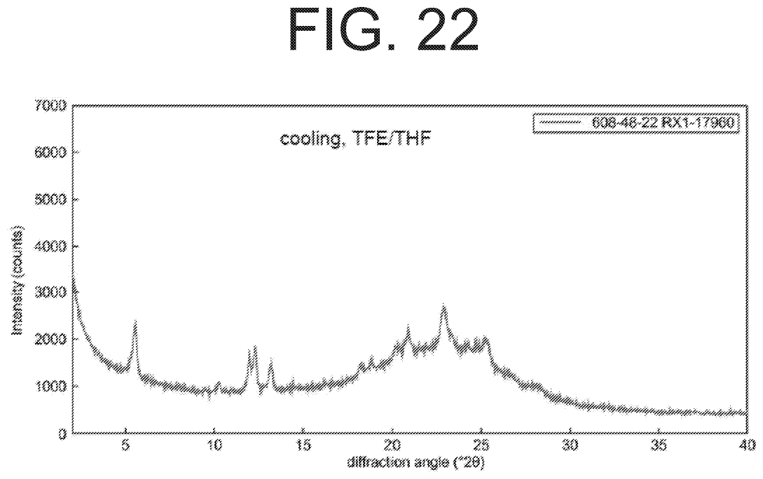

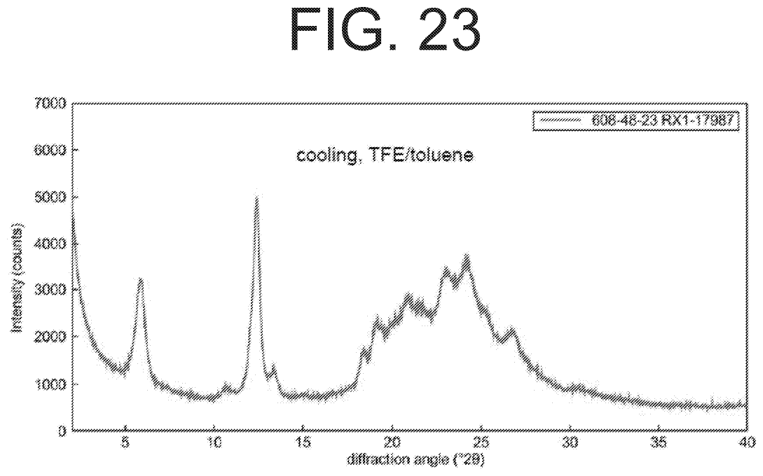

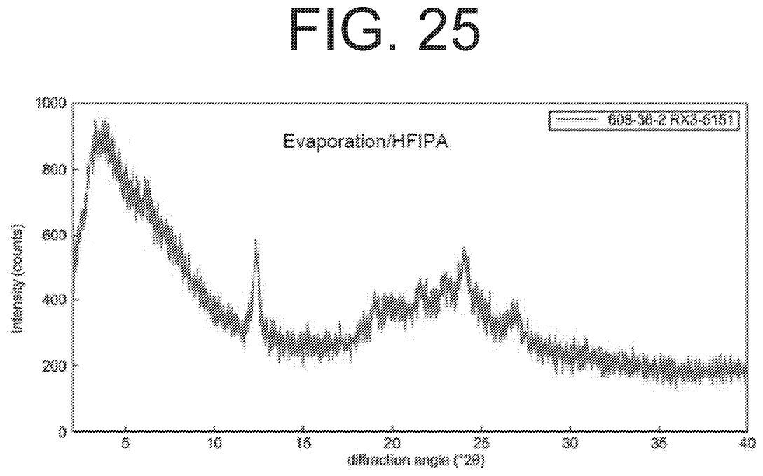

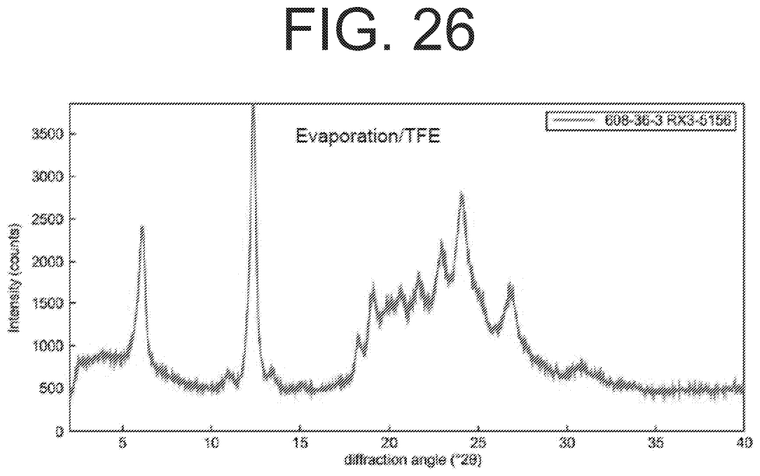

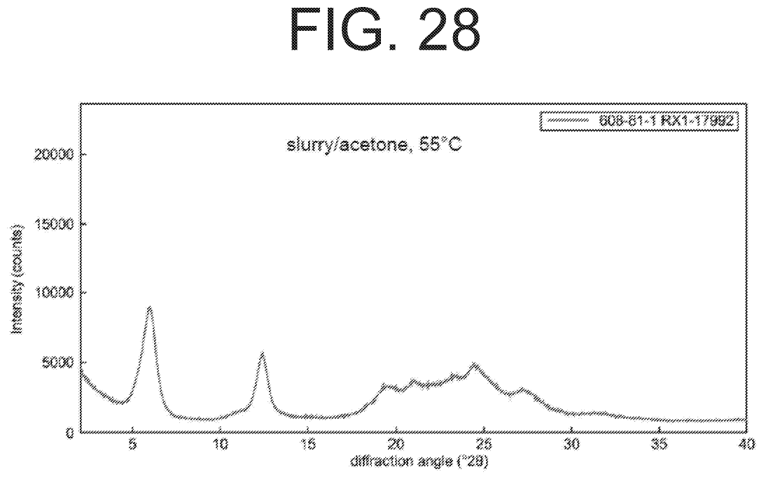

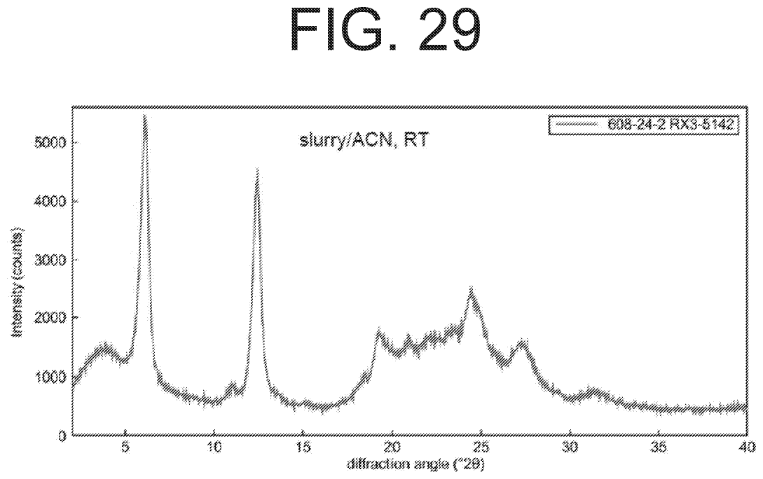

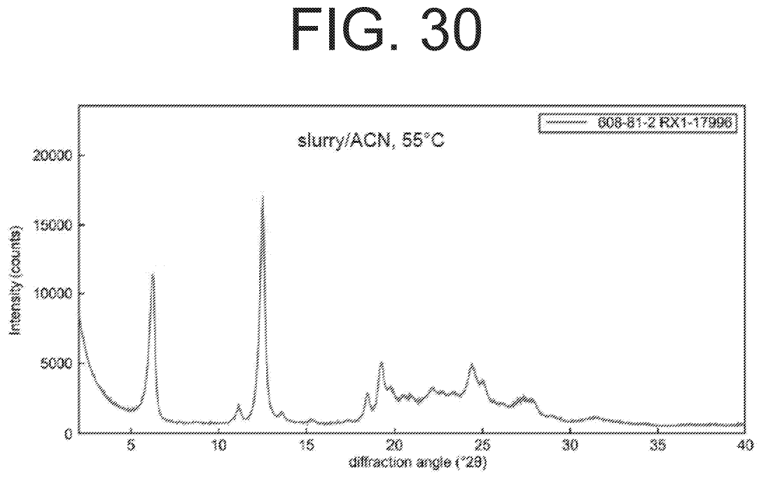

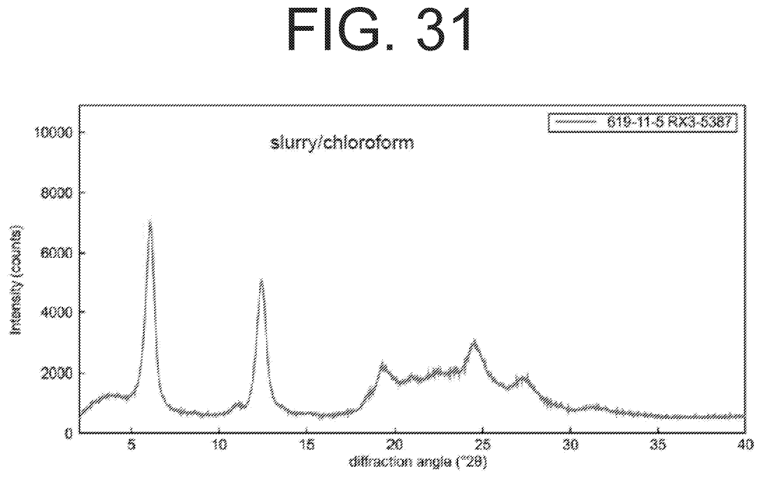

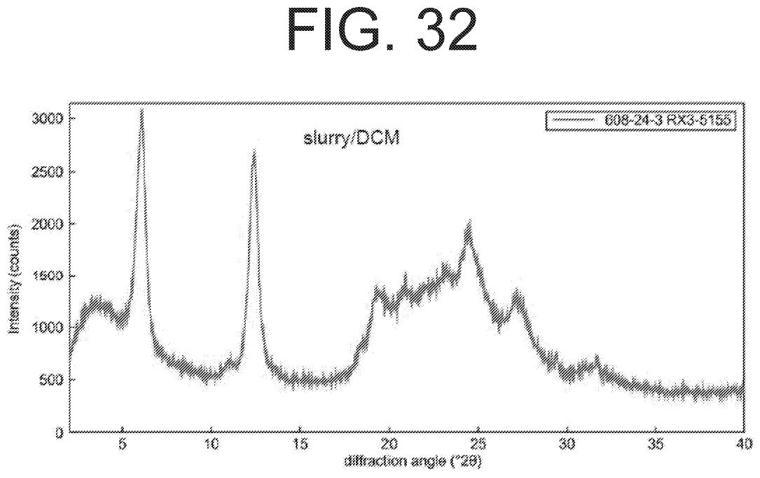

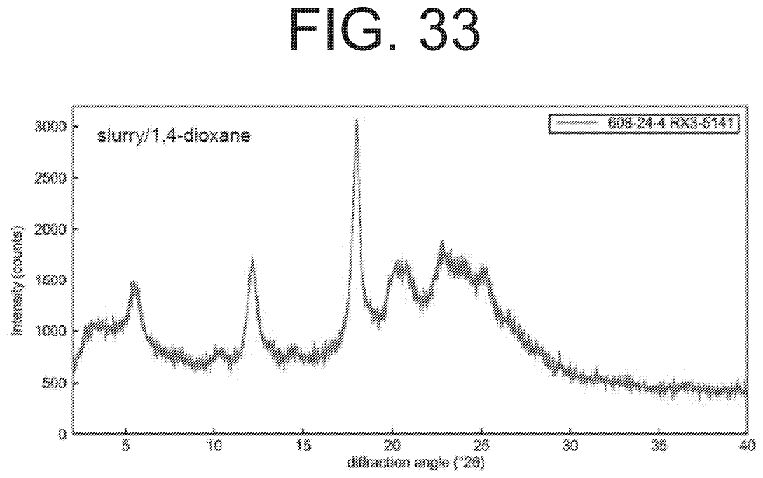

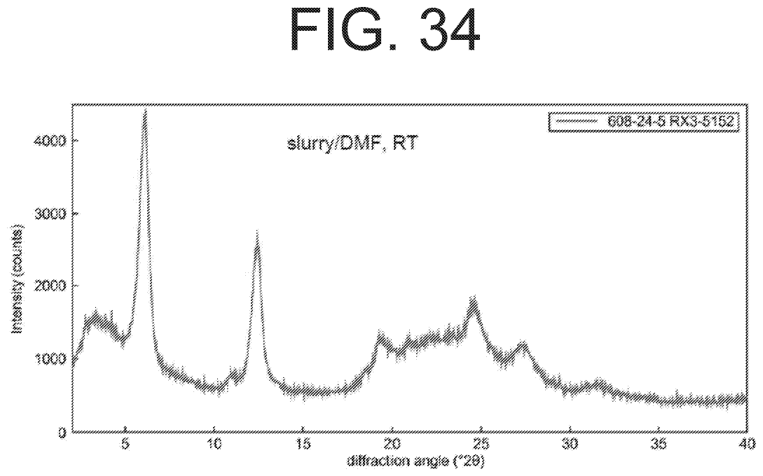

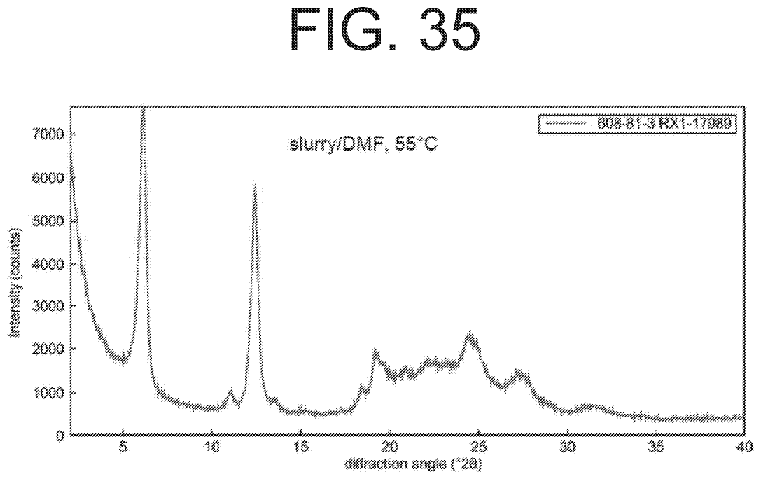

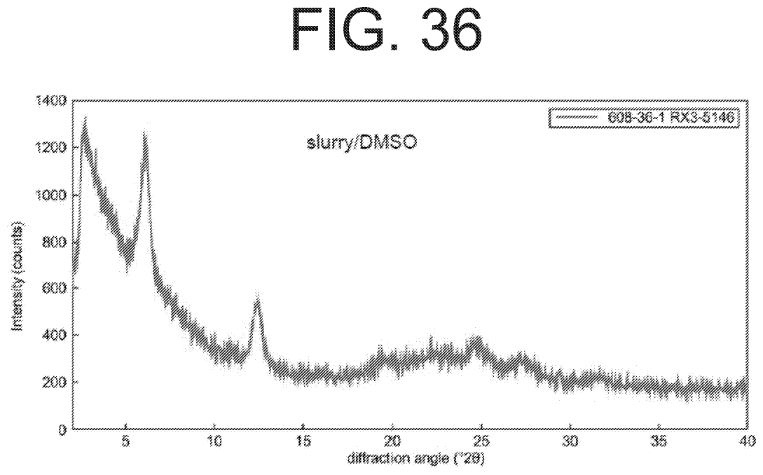

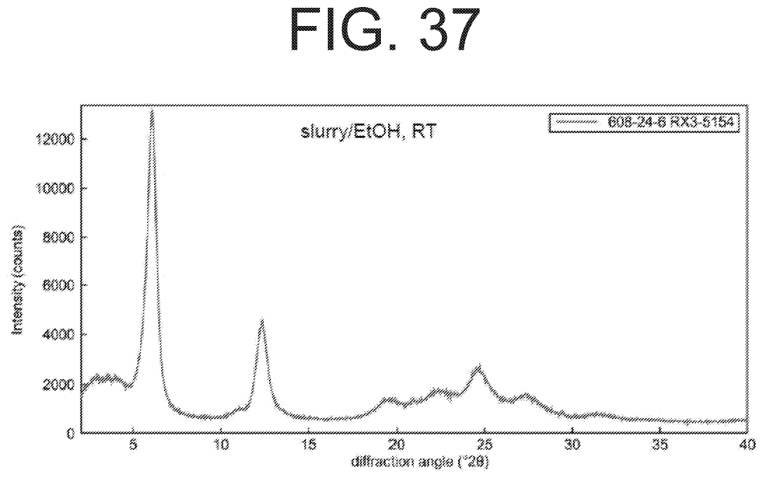

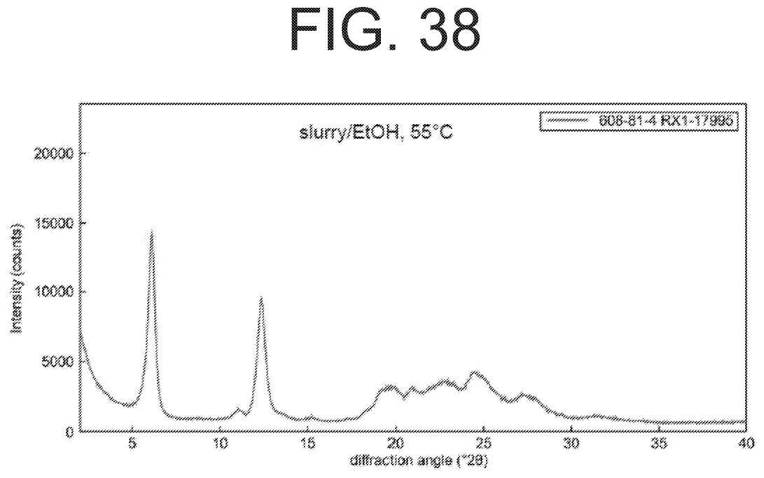

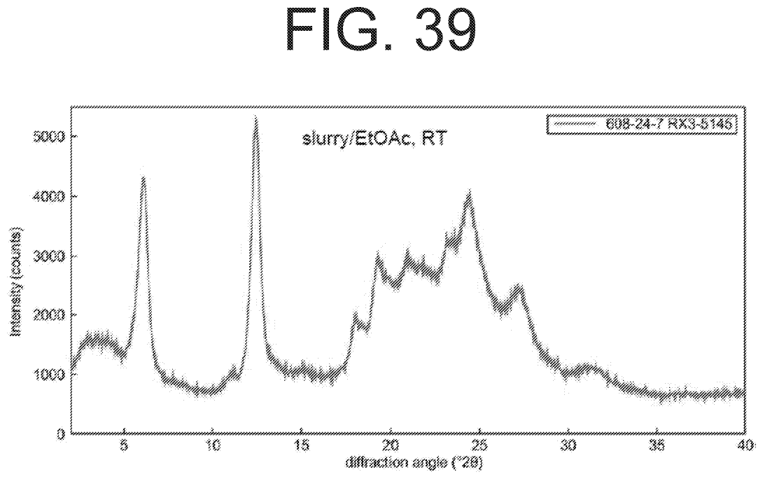

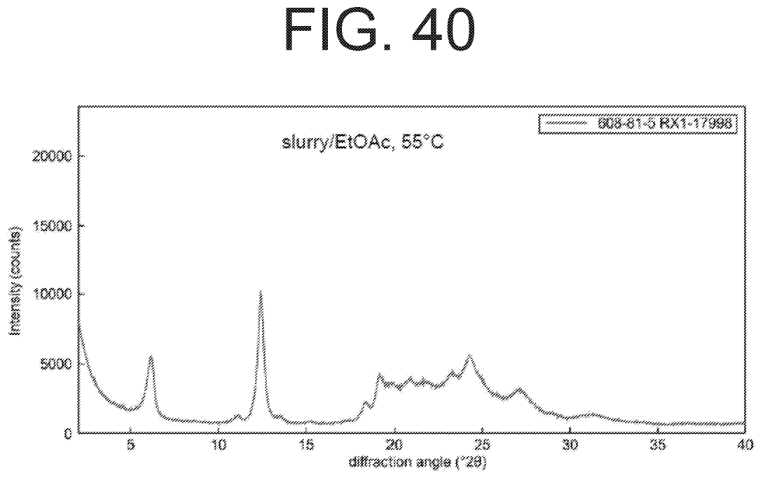

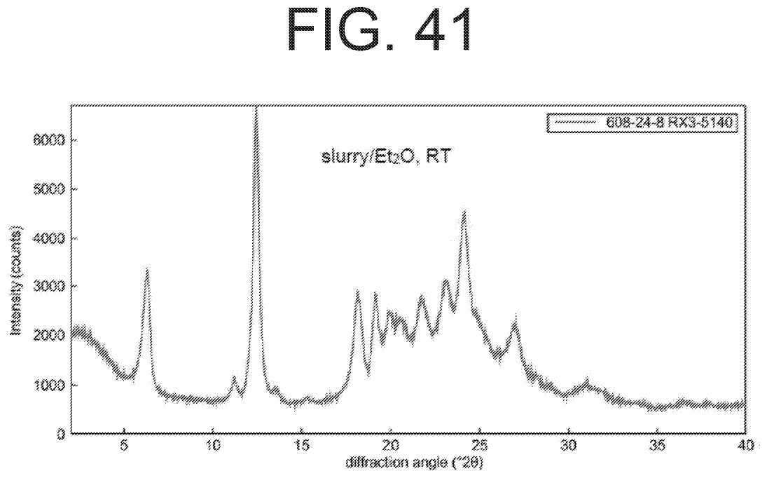

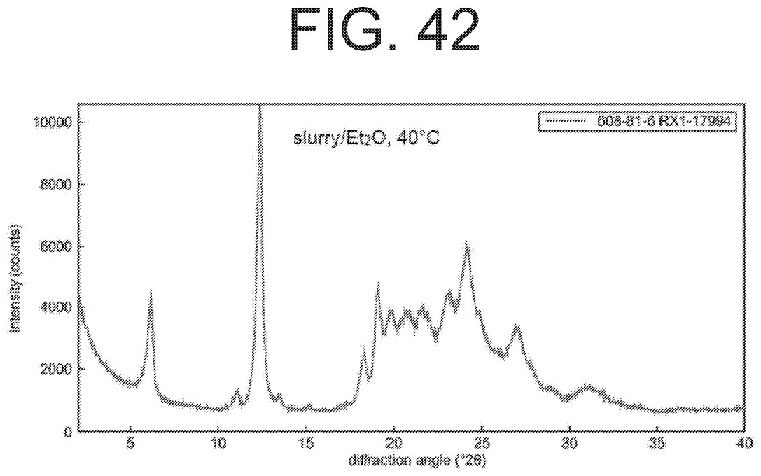

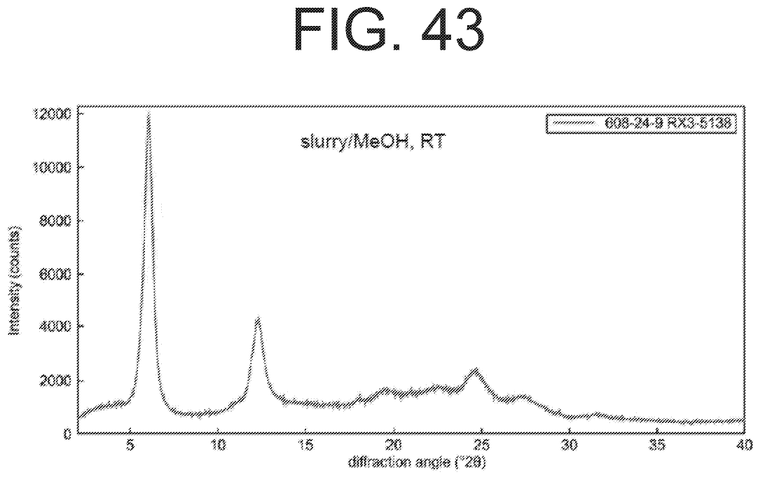

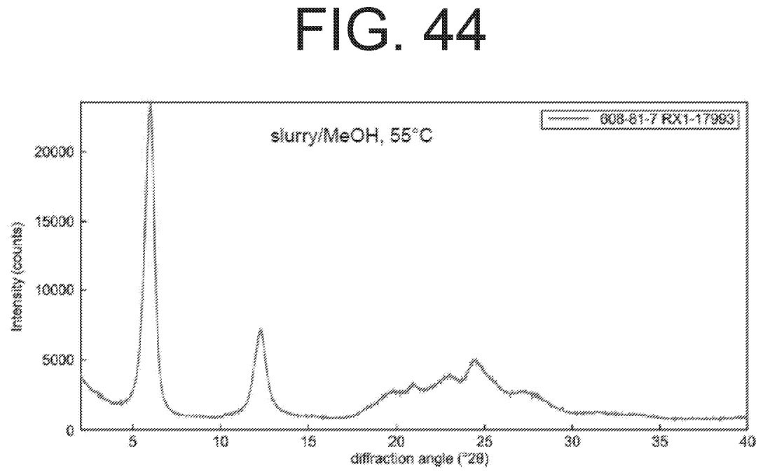

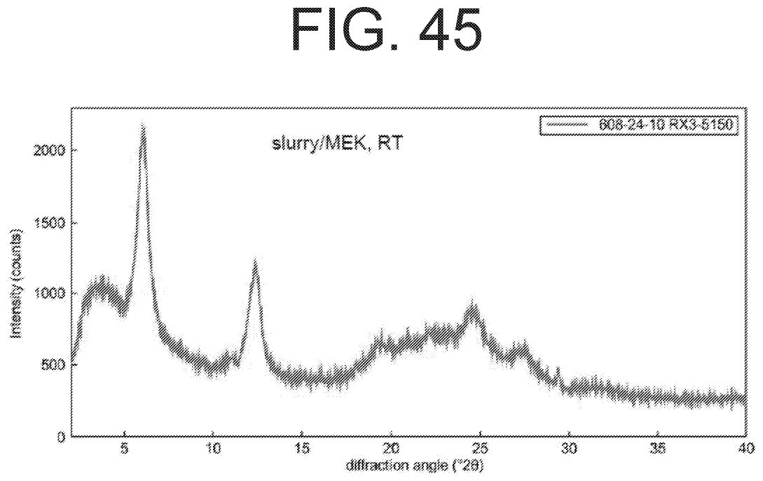

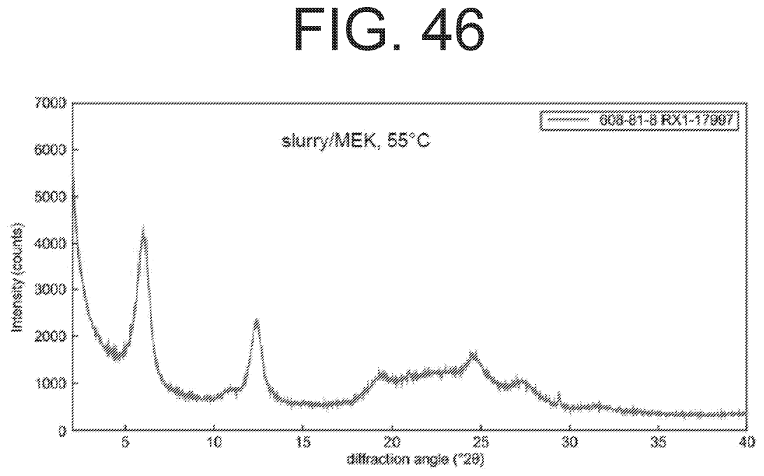

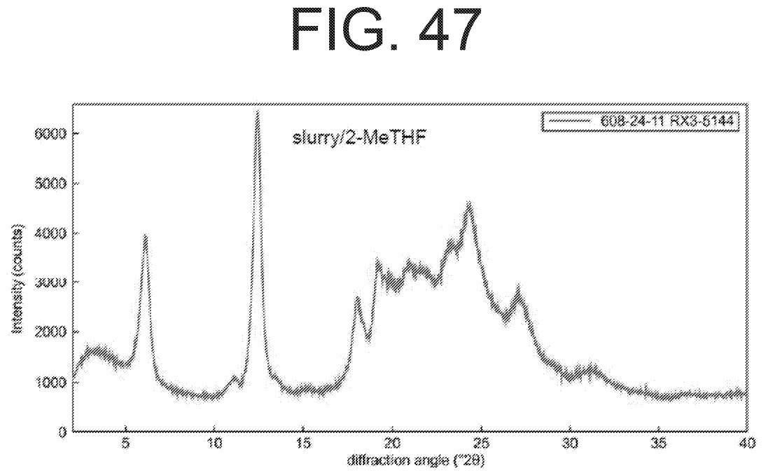

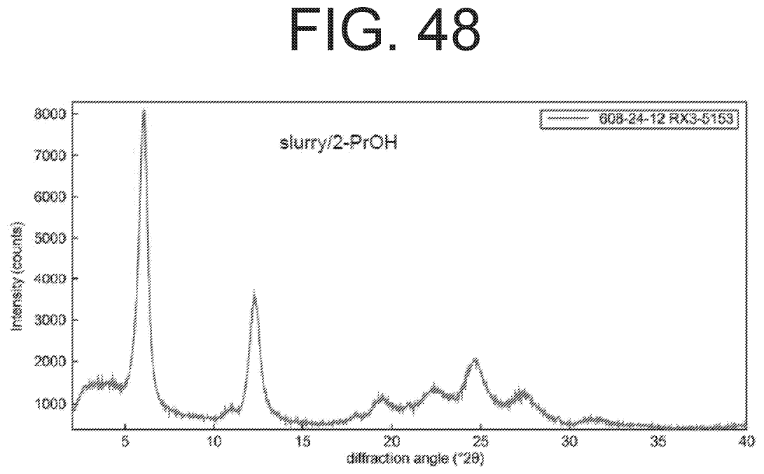

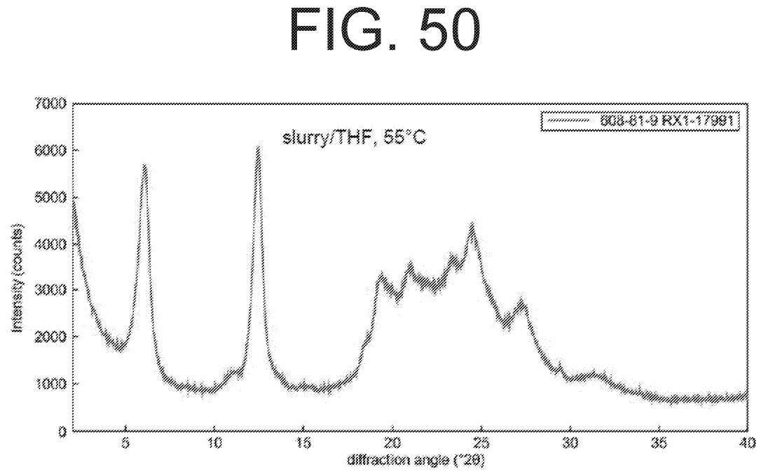

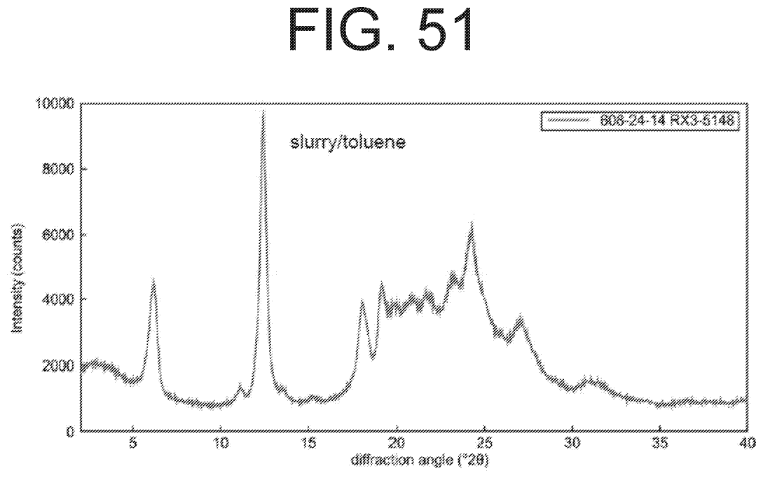

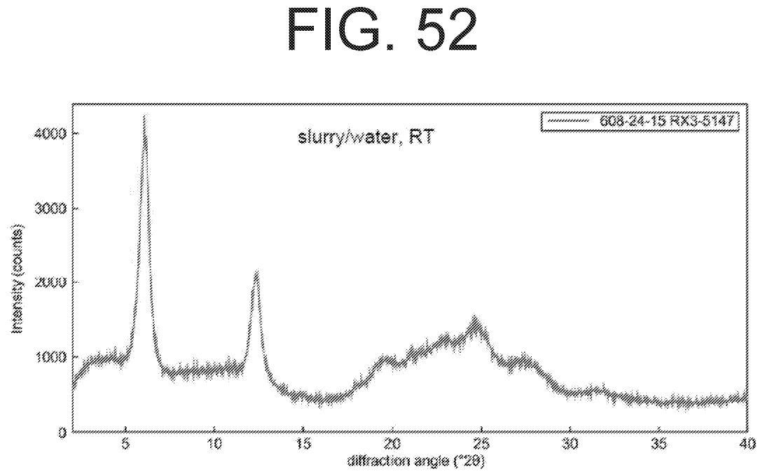

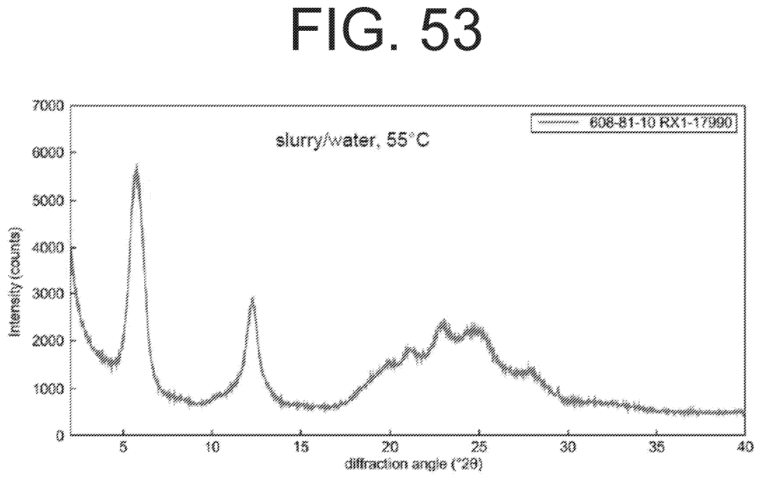

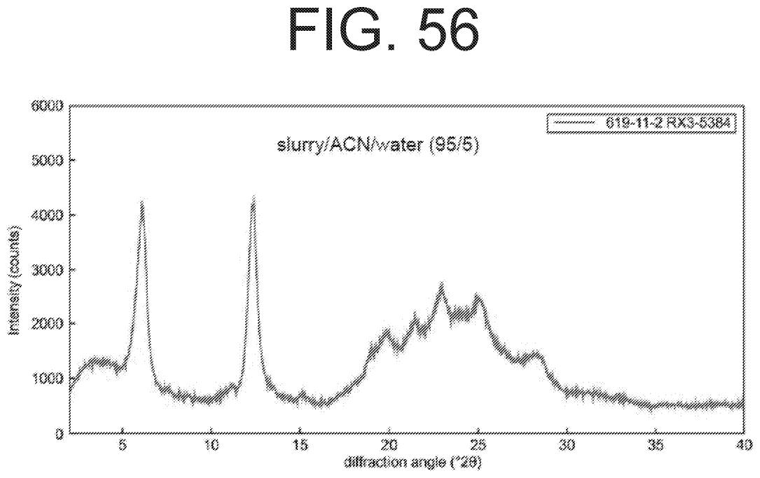

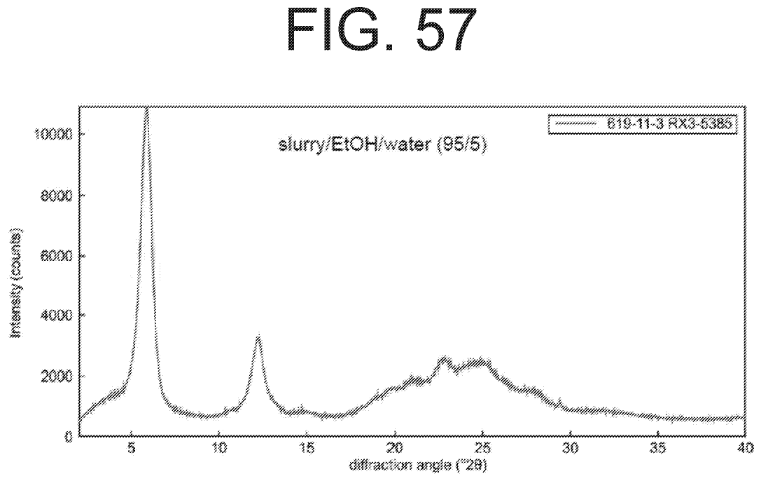

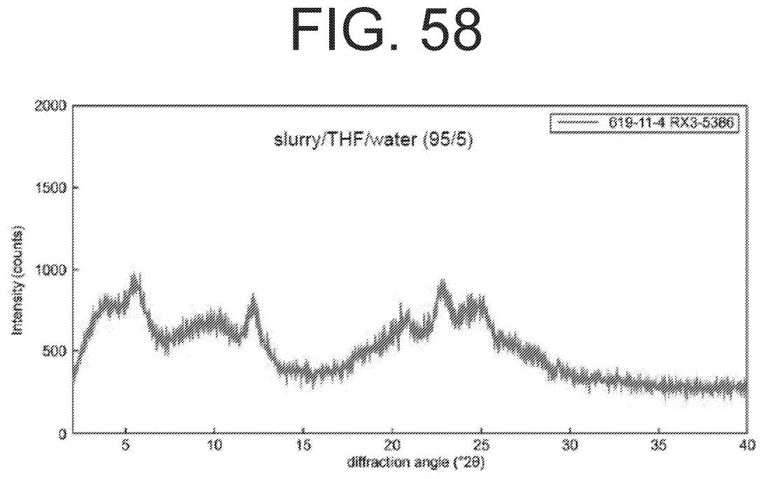

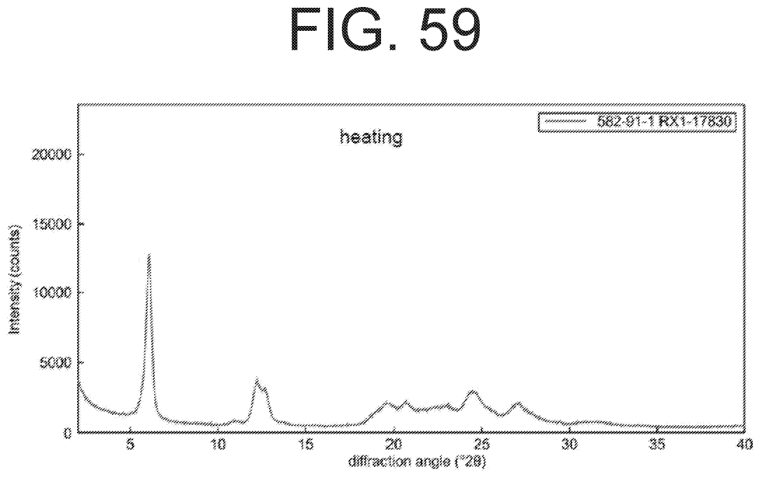

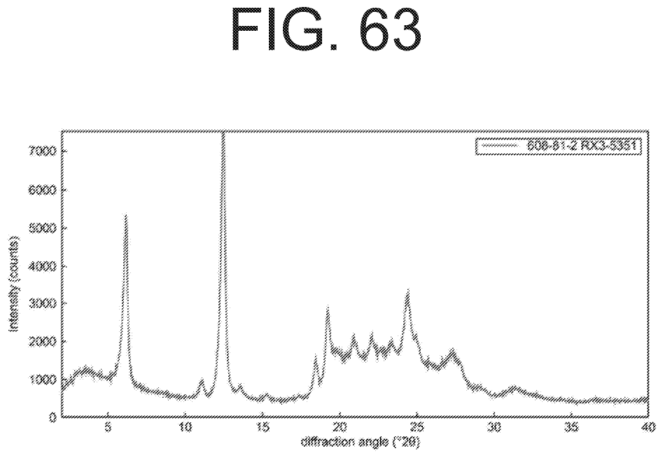

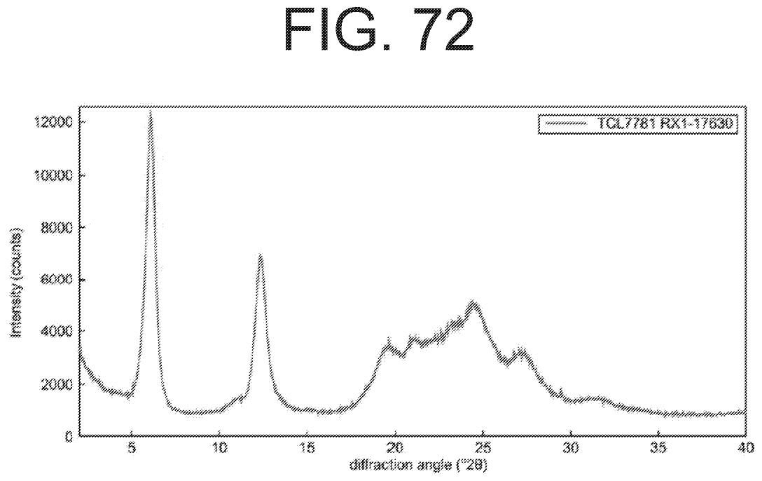

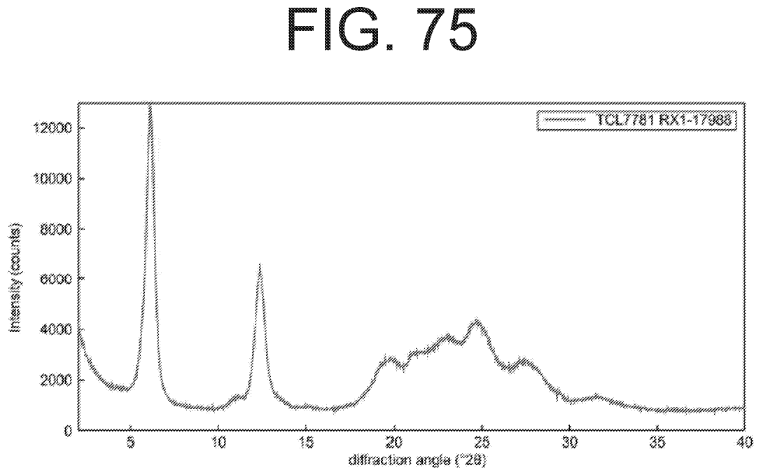

In some embodiments, the Compound of Formula (I) disclosed herein is a low-crystalline solid referred to herein as “Form 1”. In some embodiments, Form 1 of Compound of Formula (I) is substantially pure. In some embodiments, Form 1 of Compound of Formula (I) is characterized by an x-ray powder diffractogram substantially as shown in FIG. 2, FIG. 4, FIG. 6, FIG. 7, FIG. 11, FIG. 13, FIG. 14, FIG. 16, FIG. 17, FIG. 18, FIG. 19, FIG. 20, FIG. 21, FIG. 22, FIG. 23, FIG. 25, FIG. 26, FIG. 28, FIG. 29, FIG. 30, FIG. 31, FIG. 32, FIG. 33, FIG. 34, FIG. 35, FIG. 36, FIG. 37, FIG. 38, FIG. 39, FIG. 40, FIG. 41, FIG. 42, FIG. 43, FIG. 44, FIG. 45, FIG. 46, FIG. 47, FIG. 48, FIG. 50, FIG. 51, FIG. 52, FIG. 53, FIG. 56, FIG. 57, FIG. 58, FIG. 59, FIG. 63, FIG. 72 and/or FIG. 75.

In some embodiments, Form 1 of Compound of Formula (I) is at least 75% pure, at least 80% pure, at least 85% pure, at least 90% pure, at least 91% pure, at least 92% pure, at least 93% pure, at least 94% pure, at least 95% pure, at least 96% pure, at least 97% pure, at least 98% pure, or at least 99% pure. In some embodiments, Form 1 of Compound of Formula (I) is at least 95% pure. In some embodiments, Form 1 of Compound of Formula (I) is at least 98% pure. In some embodiments, Form 1 of Compound of Formula (I) is 75% pure, 80% pure, 85% pure, 90% pure, 91% pure, 92% pure, 93% pure, 94% pure, 95% pure, 96% pure, 97% pure, 98% pure, or 99% pure. In some embodiments, the purity of Form 1 is determined by LC.

In some embodiments, Form 1 of Compound of Formula (I) is characterized by an x-ray powder diffractogram having a signal at at least one, at least two, at least three, at least four, at least five, at least six, at least seven, at least eight, at least nine, at least ten, at least eleven, at least twelve, at least thirteen, at least fourteen, or at least fifteen two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2.

In some embodiments, Form 1 of Compound of Formula (I) is characterized by an x-ray powder diffractogram having a signal at at least one two-theta value, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least two two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least three two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least four two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least five two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2.

In some embodiments, Form 1 of Compound of Formula (I) is characterized by an x-ray powder diffractogram having a signal at at least six two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least seven two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least eight two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least nine two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least ten two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least eleven two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least twelve two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2.

In some embodiments, Form 1 of Compound of Formula (I) is characterized by an x-ray powder diffractogram having a signal at at least thirteen two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at at least fourteen two-theta values, ±0.2, chosen from 6.2, 11.1, 12.5, 13.6, 15.3, 18.5, 19.3, 19.8, 20.9, 22.1, 24.4, 25.0, 27.2, 27.8, and 31.2. In some embodiments, Form 1 of Compound of Formula (I) is characterized by an x-ray powder diffractogram having a signal at fifteen two-theta values, ±0.2, comprising 6.2, 11.1, 12.5, 13.6, 15.3, 18.5, 19.3, 19.8, 20.9, 22.1, 24.4, 25.0, 27.2, 27.8, and 31.2.

In some embodiments, Form 1 of Compound of Formula (I) is characterized by an x-ray powder diffractogram having a signal at at least one, at least two, at least three, at least four, at least five, at least six, at least seven, or at least eight two-theta values, ±0.2, chosen from 3.6, 6.1, 10.9, 12.5, 19.3, 20.8, 24.5, and 27.3.

In some embodiments, Form 1 of Compound of Formula (I) is characterized by an x-ray powder diffractogram having a signal at at least one two-theta value, ±0.2, chosen from 3.6, 6.1, 10.9, 12.5, 19.3, 20.8, 24.5, and 27.3. In some embodiments, Form 1 of Compound of Formula (I) is characterized by an x-ray powder diffractogram having a signal at at least two two-theta values, ±0.2, chosen from 3.6, 6.1, 10.9, 12.5, 19.3, 20.8, 24.5, and 27.3. In some embodiments, Form 1 of Compound of Formula (I) is characterized by an x-ray powder diffractogram having a signal at at least three two-theta values, ±0.2, chosen from 3.6, 6.1, 10.9, 12.5, 19.3, 20.8, 24.5, and 27.3. In some embodiments, Form 1 of Compound of Formula (I) is characterized by an x-ray powder diffractogram having a signal at at least four two-theta values, ±0.2, chosen from 3.6, 6.1, 10.9, 12.5, 19.3, 20.8, 24.5, and 27.3. In some embodiments, Form 1 of Compound of Formula (I) is characterized by an x-ray powder diffractogram having a signal at at least five two-theta values, ±0.2, chosen from 3.6, 6.1, 10.9, 12.5, 19.3, 20.8, 24.5, and 27.3.

In some embodiments, Form 1 of Compound of Formula (I) is characterized by an x-ray powder diffractogram having a signal at at least six two-theta values, ±0.2, chosen from 3.6, 6.1, 10.9, 12.5, 19.3, 20.8, 24.5, and 27.3. In some embodiments, Form 1 of Compound of Formula (I) is characterized by an x-ray powder diffractogram having a signal at at least seven two-theta values, ±0.2, chosen from 3.6, 6.1, 10.9, 12.5, 19.3, 20.8, 24.5, and 27.3. In some embodiments, Form 1 of Compound of Formula (I) is characterized by an x-ray powder diffractogram having a signal at eight two-theta values, ±0.2, comprising 3.6, 6.1, 10.9, 12.5, 19.3, 20.8, 24.5, and 27.3.

In some embodiments, Form 1 of Compound of Formula (I) is characterized by an x-ray powder diffractogram having a signal at at least one, at least two, at least three, at least four, at least five, at least six, at least seven, or at least eight two-theta values, ±0.2, chosen from 6.1, 11.4, 12.4, 19.8, 21.3, 22.8, 25.1, and 28.4.

In some embodiments, Form 1 of Compound of Formula (I) is characterized by an x-ray powder diffractogram having a signal at at least one two-theta value, ±0.2, chosen from 6.1, 11.4, 12.4, 19.8, 21.3, 22.8, 25.1, and 28.4. In some embodiments, Form 1 of Compound of Formula (I) is characterized by an x-ray powder diffractogram having a signal at at least two two-theta values, ±0.2, chosen from 6.1, 11.4, 12.4, 19.8, 21.3, 22.8, 25.1, and 28.4. In some embodiments, Form 1 of Compound of Formula (I) is characterized by an x-ray powder diffractogram having a signal at at least three two-theta values, ±0.2, chosen from 6.1, 11.4, 12.4, 19.8, 21.3, 22.8, 25.1, and 28.4. In some embodiments, Form 1 of Compound of Formula (I) is characterized by an x-ray powder diffractogram having a signal at at least four two-theta values, ±0.2, chosen from 6.1, 11.4, 12.4, 19.8, 21.3, 22.8, 25.1, and 28.4. In some embodiments, Form 1 of Compound of Formula (I) is characterized by an x-ray powder diffractogram having a signal at at least five two-theta values, ±0.2, chosen from 6.1, 11.4, 12.4, 19.8, 21.3, 22.8, 25.1, and 28.4.

In some embodiments, Form 1 of Compound of Formula (I) is characterized by an x-ray powder diffractogram having a signal at at least six two-theta values, ±0.2, chosen from 6.1, 11.4, 12.4, 19.8, 21.3, 22.8, 25.1, and 28.4. In some embodiments, Form 1 of Compound of Formula (I) is characterized by an x-ray powder diffractogram having a signal at at least seven two-theta values, ±0.2, chosen from 6.1, 11.4, 12.4, 19.8, 21.3, 22.8, 25.1, and 28.4. In some embodiments, Form 1 of Compound of Formula (I) is characterized by an x-ray powder diffractogram having a signal at eight two-theta values, ±0.2, comprising 6.1, 11.4, 12.4, 19.8, 21.3, 22.8, 25.1, and 28.4.

In some embodiments, Form 1 of Compound of Formula (I) is characterized by an x-ray powder diffractogram having a signal at at least one, at least two, or at least three two-theta values, ±0.2, chosen from 6.1, 21.3, and 22.8.

In some embodiments, Form 1 of Compound of Formula (I) is characterized by an x-ray powder diffractogram having a signal at at least one two-theta value, ±0.2, chosen from 6.1, 21.3, and 22.8. In some embodiments, Form 1 of Compound of Formula (I) is characterized by an x-ray powder diffractogram having a signal at at least two two-theta values, ±0.2, chosen from 6.1, 21.3, and 22.8. In some embodiments, Form 1 of Compound of Formula (I) is characterized by an x-ray powder diffractogram having a signal at three two-theta values, ±0.2, comprising 6.1, 21.3, and 22.8.

In some embodiments, Form 1 of Compound of Formula (I) may be produced by cooling a mixture of at least one solvent, at least one anti-solvent, and Compound of Formula (I). In some embodiments, the mixture is cooled from 60° C. to −15° C. In some embodiments, the at least one solvent is 1,1,1,3,3,3-hexafluoro-2-propanol. In some embodiments, the at least one solvent is 2,2,2-trifluorethanol. In some embodiments, the at least one anti-solvent is chosen from acetone, acetonitrile, ethanol, ethyl acetate, methanol, methyl ethyl ketone, 2-methyltetrahydrofuran, 2-propanol, tetrahydrofuran, and toluene.

In some embodiments, the at least one solvent is 1,1,1,3,3,3-hexafluoro-2-propanol and the at least one anti-solvent is acetonitrile. In some embodiments, the at least one solvent is 1,1,1,3,3,3-hexafluoro-2-propanol and the at least one anti-solvent is ethanol. In some embodiments, the at least one solvent is 1,1,1,3,3,3-hexafluoro-2-propanol and the at least one anti-solvent is methanol. In some embodiments, the at least one solvent is 1,1,1,3,3,3-hexafluoro-2-propanol and the at least one anti-solvent is methyl ethyl ketone. In some embodiments, the at least one solvent is 1,1,1,3,3,3-hexafluoro-2-propanol and the at least one anti-solvent is toluene. In some embodiments, the at least one solvent is 2,2,2-trifluorethanol and the at least one anti-solvent is acetone. In some embodiments, the at least one solvent is 2,2,2-trifluorethanol and the at least one anti-solvent is acetonitrile. In some embodiments, the at least one solvent is 2,2,2-trifluorethanol and the at least one anti-solvent is ethanol. In some embodiments, the at least one solvent is 2,2,2-trifluorethanol and the at least one anti-solvent is ethyl acetate. In some embodiments, the at least one solvent is 2,2,2-trifluorethanol and the at least one anti-solvent is methanol. In some embodiments, the at least one solvent is 2,2,2-trifluorethanol and the at least one anti-solvent is methyl ethyl ketone. In some embodiments, the at least one solvent is 2,2,2-trifluorethanol and the at least one anti-solvent is 2-methyltetrahydrofuran. In some embodiments, the at least one solvent is 2,2,2-trifluorethanol and the at least one anti-solvent is 2-propanol. In some embodiments, the at least one solvent is 2,2,2-trifluorethanol and the at least one anti-solvent is tetrahydrofuran. In some embodiments, the at least one solvent is 2,2,2-trifluorethanol and the at least one anti-solvent is toluene.

In some embodiments, Form 1 of Compound of Formula (I) is produced by evaporating at least one solvent. In some embodiments, the evaporation is at room temperature. In some embodiments, the at least one solvent is 1,1,1,3,3,3-hexafluoro-2-propanol. In some embodiments, the at least one solvent is 2,2,2-trifluorethanol.

In some embodiments, Form 1 of Compound of Formula (I) is produced by slurrying a mixture of at least one solvent and Compound of Formula (I). In some embodiments, the mixture is slurried at room temperature. In some embodiments, the mixture is slurried at 55° C. In some embodiments, the mixture is slurried for seven days. In some embodiments, the at least one solvent is chosen from acetone, acetonitrile, chloroform, dichloromethane, dioxane, N,N-dimethylformamide, dimethylsulfoxide, ethanol, ethyl acetate, diethyl ether, methanol, methyl ethyl ketone, 2-methyltetrahydrofuran, 2-propanol, tetrahydrofuran, toluene, water, acetone/water (95/5, v/v), ethanol/water (95/5, v/v), and THF/water (95/5, v/v).

In some embodiments, the at least one solvent is acetone and the mixture is slurried at 55° C. for seven days. In some embodiments, the at least one solvent is acetonitrile and the mixture is slurried at room temperature for seven days. In some embodiments, the at least one solvent is acetonitrile and the mixture is slurried at 55° C. for seven days. In some embodiments, the at least one solvent is chloroform and the mixture is slurried at room temperature for seven days. In some embodiments, the at least one solvent is dichloromethane and the mixture is slurried at room temperature for seven days. In some embodiments, the at least one solvent is dioxane and the mixture is slurried at room temperature for seven days. In some embodiments, the at least one solvent is N,N-dimethylformamide and the mixture is slurried at room temperature for seven days. In some embodiments, the at least one solvent is N,N-dimethylformamide and the mixture is slurried at 55° C. for seven days. In some embodiments, the at least one solvent is dimethylsulfoxide and the mixture is slurried at room temperature for seven days. In some embodiments, the at least one solvent is ethanol and the mixture is slurried at room temperature for seven days. In some embodiments, the at least one solvent is ethanol and the mixture is slurried at 55° C. for seven days. In some embodiments, the at least one solvent is ethyl acetate and the mixture is slurried at room temperature for seven days. In some embodiments, the at least one solvent is ethyl acetate and the mixture is slurried at 55° C. for seven days. In some embodiments, the at least one solvent is diethyl ether and the mixture is slurried at room temperature for seven days. In some embodiments, the at least one solvent is diethyl ether and the mixture is slurried at 55° C. for seven days. In some embodiments, the at least one solvent is methanol and the mixture is slurried at room temperature for seven days. In some embodiments, the at least one solvent is methanol and the mixture is slurried at 55° C. for seven days. In some embodiments, the at least one solvent is methyl ethyl ketone and the mixture is slurried at room temperature for seven days. In some embodiments, the at least one solvent is methyl ethyl ketone and the mixture is slurried at 55° C. for seven days. In some embodiments, the at least one solvent is 2-methyltetrahydrofuran and the mixture is slurried at room temperature for seven days. In some embodiments, the at least one solvent is 2-propanol and the mixture is slurried at room temperature for seven days. In some embodiments, the at least one solvent is tetrahydrofuran and the mixture is slurried at 55° C. for seven days. In some embodiments, the at least one solvent is toluene and the mixture is slurried at room temperature for seven days. In some embodiments, the at least one solvent is water and the mixture is slurried at room temperature for seven days. In some embodiments, the at least one solvent is water and the mixture is slurried at 55° C. for seven days. In some embodiments, the at least one solvent is acetonitrile/water (95/5, v/v) and the mixture is slurried at room temperature for seven days. In some embodiments, the at least one solvent is ethanol/water (95/5, v/v) and the mixture is slurried at room temperature for seven days. In some embodiments, the at least one solvent is tetrahydrofuran/water (95/5, v/v) and the mixture is slurried at room temperature for seven days.

In some embodiments, Form 1 of Compound of Formula (I) is produced by heating Compound of Formula (I). In some embodiments, the Compound of Formula (I) is heated to at least 100° C., at least 110° C., at least 120° C., at least 130° C., at least 140° C., at least 150° C., or at least 160° C. to less than 250° C. In some embodiments, the Compound of Formula (I) is heated at 100° C., at 110° C., at 120° C., at 130° C., at 140° C., at 150° C., or at 160° C. In some embodiments, the Compound of Formula (I) is heated at 145° C. In some embodiments, the Compound of Formula (I) is heated for at least 10 seconds, at least 20 seconds, at least 30 seconds, at least 40 seconds, at least 50 seconds, at least 60 seconds, at least 70 seconds, at least 80 seconds, at least 90 seconds, at least 100 seconds, at least 110 seconds, at least 120 seconds, at least 150 seconds, at least 180 seconds, at least 210 seconds, at least 240 seconds, at least 270 seconds, at least 5 minutes, at least 6 minutes, at least 7 minutes, at least 8 minutes, at least 9 minutes, or at least 10 minutes to less than 15 minutes. In some embodiments, the Compound of Formula (I) is heated for 10 seconds, 20 seconds, 30 seconds, 40 seconds, 50 seconds, 60 seconds, 70 seconds, 80 seconds, 90 seconds, 100 seconds, 110 seconds, 120 seconds, 150 seconds, 180 seconds, 210 seconds, 240 seconds, 270 seconds, 5 minutes, 6 minutes, 7 minutes, 8 minutes, 9 minutes, or 10 minutes. In some embodiments, the Compound of Formula (I) is heated for 1 minute.

In some embodiments, provided herein is a pharmaceutical composition comprising Compound of Formula (I) formed from combining Form 1 of Compound of Formula (I) and at least one additional component chosen from pharmaceutically acceptable carriers, pharmaceutically acceptable vehicles, and pharmaceutically acceptable excipients. In some embodiments, provided herein is a pharmaceutical composition consisting of Compound of Formula (I) formed from combining Form 1 of Compound of Formula (I) and at least one additional component chosen from pharmaceutically acceptable carriers, pharmaceutically acceptable vehicles, and pharmaceutically acceptable excipients. In some embodiments, provided herein is a pharmaceutical composition consisting essentially of Compound of Formula (I) formed from combining Form 1 of Compound of Formula (I) and at least one additional component chosen from pharmaceutically acceptable carriers, pharmaceutically acceptable vehicles, and pharmaceutically acceptable excipients.

Form 2 of Compound of Formula (I)

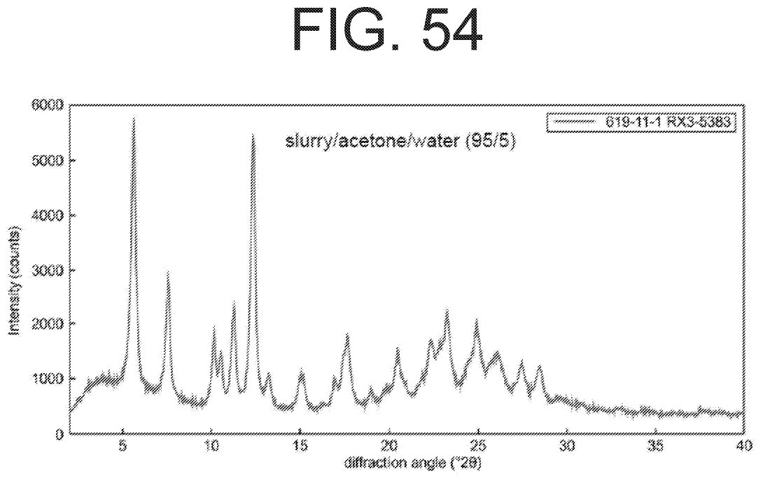

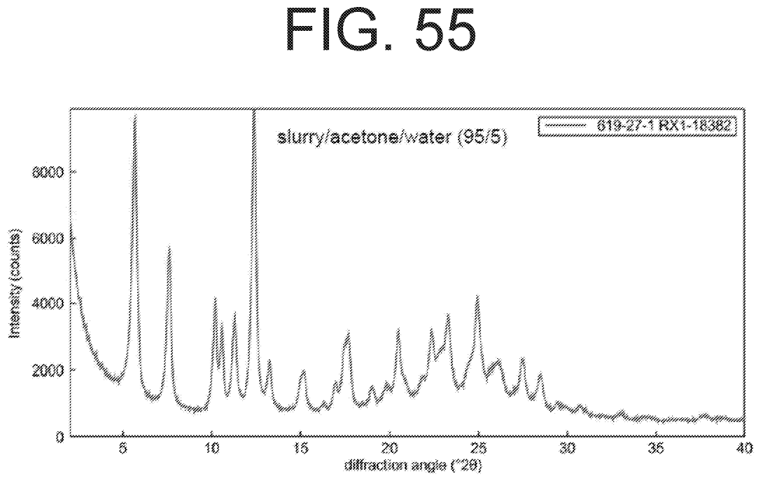

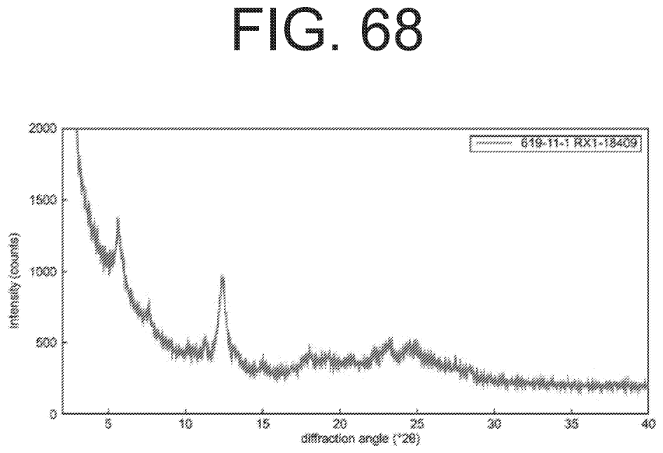

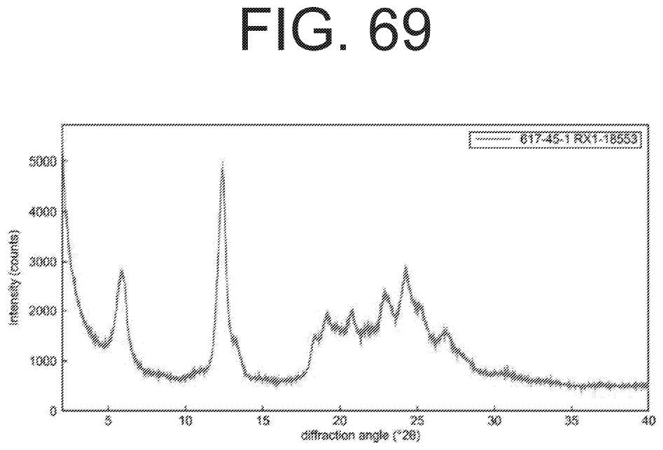

In some embodiments, Compound of Formula (I) disclosed herein is in the form of a crystalline solid. In some embodiments, the Compound of Formula (I) disclosed herein is in the form of Form 2. In some embodiments, Form 2 of the Compound of Formula (I) is characterized by an x-ray powder diffractogram substantially as shown in FIG. 54, FIG. 55, FIG. 68, and/or FIG. 69.

In some embodiments, Form 2 of Compound of Formula (I) is at least 75% pure, at least 80% pure, at least 85% pure, at least 90% pure, at least 91% pure, at least 92% pure, at least 93% pure, at least 94% pure, at least 95% pure, at least 96% pure, at least 97% pure, at least 98% pure, or at least 99% pure. In some embodiments, Form 2 of Compound of Formula (I) is at least 95% pure. In some embodiments, Form 2 of Compound of Formula (I) is at least 98% pure. In some embodiments, Form 2 of Compound of Formula (I) is 75% pure, 80% pure, 85% pure, 90% pure, 91% pure, 92% pure, 93% pure, 94% pure, 95% pure, 96% pure, 97% pure, 98% pure, or 99% pure. In some embodiments, Form 2 of Compound of Formula (I) is 95% pure. In some embodiments, Form 2 of Compound of Formula (I) is 98% pure. In some embodiments, the purity of Form 2 is determined by LC.

In some embodiments, Form 2 of Compound of Formula (I) is characterized by an x-ray powder diffractogram having a signal at at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or nineteen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4.

In some embodiments, Form 2 of Compound of Formula (I) is characterized by an x-ray powder diffractogram having a signal at at least one two-theta value, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least two two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least three two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least four two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least five two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4.

In some embodiments, Form 2 of Compound of Formula (I) is characterized by an x-ray powder diffractogram having a signal at at least six two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least seven two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least eight two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least nine two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least ten two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4.

In some embodiments, Form 2 of Compound of Formula (I) is characterized by an x-ray powder diffractogram having a signal at at least eleven two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least twelve two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least thirteen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least fourteen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least fifteen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4.

In some embodiments, Form 2 of Compound of Formula (I) is characterized by an x-ray powder diffractogram having a signal at at least sixteen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least seventeen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at at least eighteen two-theta values, ±0.2, chosen from 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4. In some embodiments, Form 2 of Compound of Formula (I) is characterized by an x-ray powder diffractogram having a signal at nineteen two-theta values, ±0.2, comprising 5.6, 7.6, 10.2, 10.6, 11.3, 12.4, 13.2, 15.1, 17.0, 17.7, 19.0, 19.8, 20.4, 22.3, 23.3, 24.9, 26.1, 27.4, and 28.4.

In some embodiments, Form 2 of Compound of Formula (I) is characterized by an x-ray powder diffractogram having a signal at at least one, at least two, at least three, at least four, at least five, at least six, at least seven, or eight two-theta values, ±0.2, chosen from 5.6, 7.6, 10.2, 10.6, 12.4, 15.1, 17.0, and 17.7.

In some embodiments, Form 2 of Compound of Formula (I) is characterized by an x-ray powder diffractogram having a signal at at least one two-theta value, ±0.2, chosen from 5.6, 7.6, 10.2, 10.6, 12.4, 15.1, 17.0, and 17.7. In some embodiments, Form 2 of Compound of Formula (I) is characterized by an x-ray powder diffractogram having a signal at at least two two-theta values, ±0.2, chosen from 5.6, 7.6, 10.2, 10.6, 12.4, 15.1, 17.0, and 17.7. In some embodiments, Form 2 of Compound of Formula (I) is characterized by an x-ray powder diffractogram having a signal at at least three two-theta values, ±0.2, chosen from 5.6, 7.6, 10.2, 10.6, 12.4, 15.1, 17.0, and 17.7. In some embodiments, Form 2 of Compound of Formula (I) is characterized by an x-ray powder diffractogram having a signal at at least four two-theta values, ±0.2, chosen from 5.6, 7.6, 10.2, 10.6, 12.4, 15.1, 17.0, and 17.7. In some embodiments, the Form 2 of Compound of Formula (I) is characterized by an x-ray powder diffractogram having a signal at at least five two-theta values, ±0.2, chosen from 5.6, 7.6, 10.2, 10.6, 12.4, 15.1, 17.0, and 17.7.

In some embodiments, Form 2 of Compound of Formula (I) is characterized by an x-ray powder diffractogram having a signal at at least six two-theta values, ±0.2, chosen from 5.6, 7.6, 10.2, 10.6, 12.4, 15.1, 17.0, and 17.7. In some embodiments, Form 2 of Compound of Formula (I) is characterized by an x-ray powder diffractogram having a signal at at least seven two-theta values, ±0.2, chosen from 5.6, 7.6, 10.2, 10.6, 12.4, 15.1, 17.0, and 17.7. In some embodiments, Form 2 of Compound of Formula (I) is characterized by an x-ray powder diffractogram having a signal at eight two-theta values, ±0.2, comprising 5.6, 7.6, 10.2, 10.6, 12.4, 15.1, 17.0, and 17.7.

In some embodiments, Form 2 of Compound of Formula (I) is characterized by an x-ray powder diffractogram having a signal at at least one, at least two, at least three, at least four, at least five, at least six, or seven, two-theta values, ±0.2, chosen from 5.6, 7.6, 10.2, 10.6, 12.4, 15.1, and 17.7.

In some embodiments, Form 2 of Compound of Formula (I) is characterized by an x-ray powder diffractogram having a signal at at least one two-theta value, ±0.2, chosen from 5.6, 7.6, 10.2, 10.6, 12.4, 15.1, and 17.7. In some embodiments, Form 2 of Compound of Formula (I) is characterized by an x-ray powder diffractogram having a signal at at least two two-theta values, ±0.2, chosen from 5.6, 7.6, 10.2, 10.6, 12.4, 15.1, and 17.7. In some embodiments, Form 2 of Compound of Formula (I) is characterized by an x-ray powder diffractogram having a signal at at least three two-theta values, ±0.2, chosen from 5.6, 7.6, 10.2, 10.6, 12.4, 15.1, and 17.7. In some embodiments, Form 2 of Compound of Formula (I) is characterized by an x-ray powder diffractogram having a signal at at least four two-theta values, ±0.2, chosen from 5.6, 7.6, 10.2, 10.6, 12.4, 15.1, and 17.7. In some embodiments, Form 2 of Compound of Formula (I) is characterized by an x-ray powder diffractogram having a signal at at least five two-theta values, ±0.2, chosen from 5.6, 7.6, 10.2, 10.6, 12.4, 15.1, and 17.7. In some embodiments, Form 2 of Compound of Formula (I) is characterized by an x-ray powder diffractogram having a signal at at least six two-theta values, ±0.2, chosen from 5.6, 7.6, 10.2, 10.6, 12.4, 15.1, and 17.7. In some embodiments, Form 2 of Compound of Formula (I) is characterized by an x-ray powder diffractogram having a signal at seven two-theta values, ±0.2, comprising 5.6, 7.6, 10.2, 10.6, 12.4, 15.1, and 17.7.

In some embodiments, Form 2 of Compound of Formula (I) is characterized by an x-ray powder diffractogram having a signal at at least one, at least two, at least three, at least four, or five two-theta values, ±0.2, chosen from 5.6, 7.6, 10.2, 10.6, and 12.4.

In some embodiments, Form 2 of Compound of Formula (I) is characterized by an x-ray powder diffractogram having a signal at at least one two-theta value, ±0.2, chosen from 5.6, 7.6, 10.2, 10.6, and 12.4. In some embodiments, Form 2 of Compound of Formula (I) is characterized by an x-ray powder diffractogram having a signal at at least two two-theta values, ±0.2, chosen from 5.6, 7.6, 10.2, 10.6, and 12.4. In some embodiments, Form 2 of Compound of Formula (I) is characterized by an x-ray powder diffractogram having a signal at at least three two-theta values, ±0.2, chosen from 5.6, 7.6, 10.2, 10.6, and 12.4. In some embodiments, Form 2 of Compound of Formula (I) is characterized by an x-ray powder diffractogram having a signal at at least four two-theta values, ±0.2, chosen from 5.6, 7.6, 10.2, 10.6, and 12.4. In some embodiments, Form 2 of Compound of Formula (I) is characterized by an x-ray powder diffractogram having a signal at five two-theta values, ±0.2, comprising 5.6, 7.6, 10.2, 10.6, and 12.4.

In some embodiments, Form 2 of Compound of Formula (I) is characterized by an x-ray powder diffractogram having a signal at at least one, at least two, at three two-theta values, ±0.2, chosen from 5.6, 7.6, and 12.4.

In some embodiments, Form 2 of Compound of Formula (I) is characterized by an x-ray powder diffractogram having a signal at at least one two-theta value, ±0.2, chosen from 5.6, 7.6, and 12.4. In some embodiments, Form 2 of Compound of Formula (I) is characterized by an x-ray powder diffractogram having a signal at at least two two-theta values, ±0.2, chosen from 5.6, 7.6, and 12.4. In some embodiments, Form 2 of Compound of Formula (I) is characterized by an x-ray powder diffractogram having a signal at three two-theta values, ±0.2, comprising 5.6, 7.6, and 12.4.

In some embodiments, Form 2 of Compound of Formula (I) is produced by slurrying a mixture comprising at least one solvent and the Compound of Formula (I). In some embodiments, the mixture is slurried at room temperature. In some embodiments, the mixture is slurried at at least 10° C., at least 20° C., at least 30° C., at least 40° C., at least 50° C., or at least 60° C. to less than 250° C. In some embodiments, the mixture is slurried at 10° C., at 20° C., at 30° C., at 40° C., at 50° C., or at 60° C. In some embodiments, the mixture is slurried at 55° C. In some embodiments, the mixture is slurried for at least 15 minutes, at least 30 minutes, at least 45 minutes, at least one hour, at least two hours, at least three hours, at least four hours, at least five hours, at least six hours, at least seven hours, at least eight hours, at least 12 hours, at least 16 hours, at least one day, at least two days, at least three days, at least four days, at least five days, at least six days, at least seven days, at least eight days, at least nine days, at least ten days, at least fifteen days, or at least one month to less than six months. In some embodiments, the mixture is slurried for seven days. In some embodiments, the at least one solvent is acetone. In some embodiments, the at least one solvent is acetone and the mixture is slurried for seven days at room temperature.

In some embodiments, provided herein is a pharmaceutical composition comprising a Compound of Formula (I) formed from combining Form 2 of Compound of Formula (I) and at least one additional component chosen from pharmaceutically acceptable carriers, pharmaceutically acceptable vehicles, and pharmaceutically acceptable excipients. In some embodiments, provided herein is a pharmaceutical composition consisting of a Compound of Formula (I) formed from combining Form 2 of Compound of Formula (I) and at least one additional component chosen from pharmaceutically acceptable carriers, pharmaceutically acceptable vehicles, and pharmaceutically acceptable excipients. In some embodiments, provided herein is a pharmaceutical composition consisting essentially of a Compound of Formula (I) formed from combining Form 2 of Compound of Formula (I) and at least one additional component chosen from pharmaceutically acceptable carriers, pharmaceutically acceptable vehicles, and pharmaceutically acceptable excipients.

Non-Crystalline Solid Form of Compound of Formula (I)

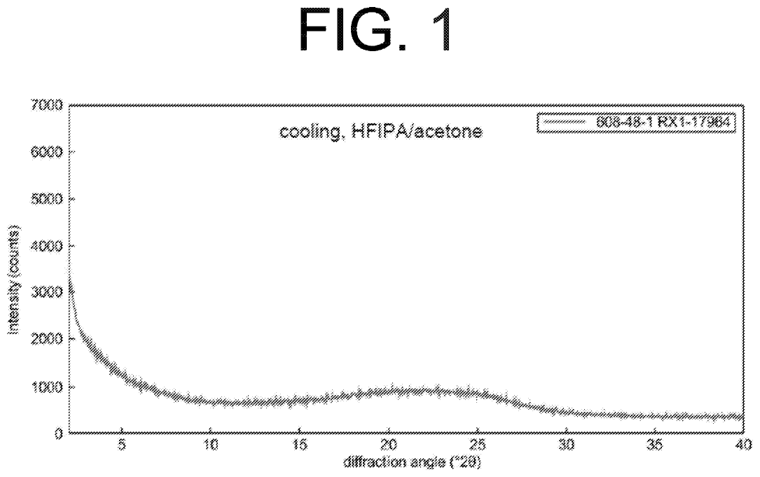

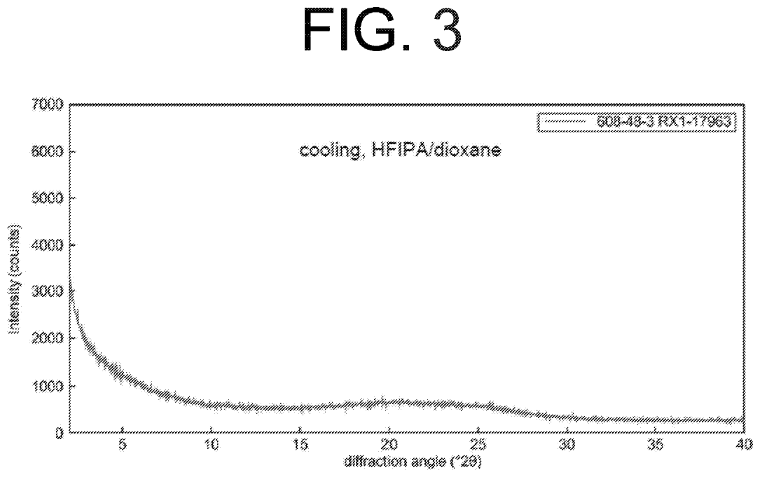

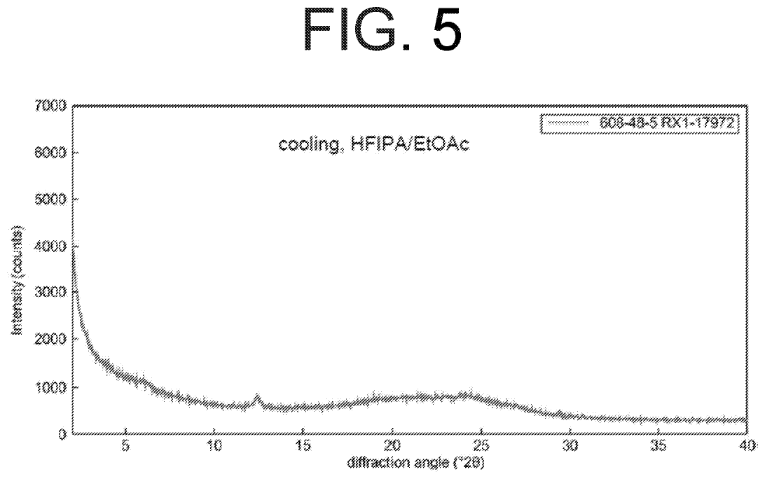

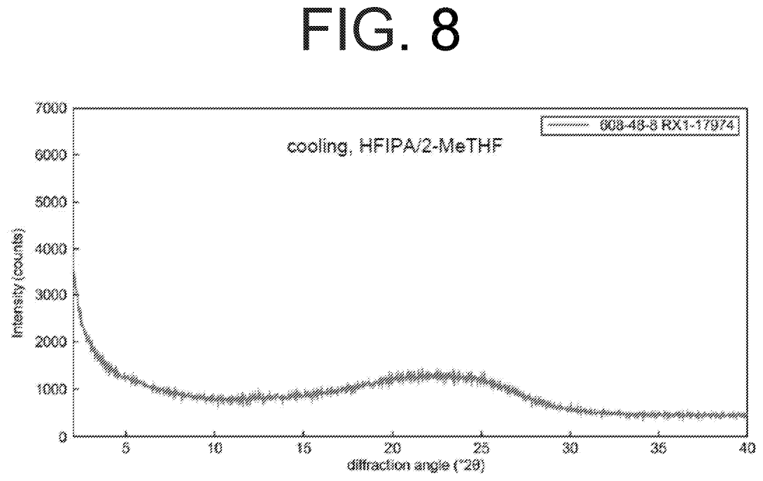

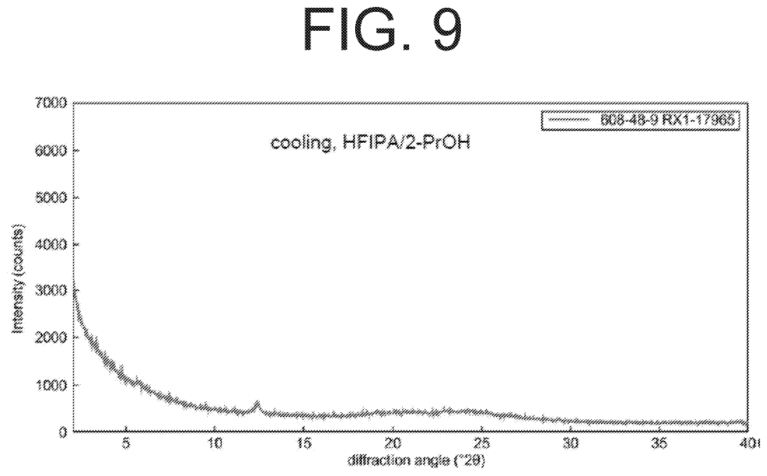

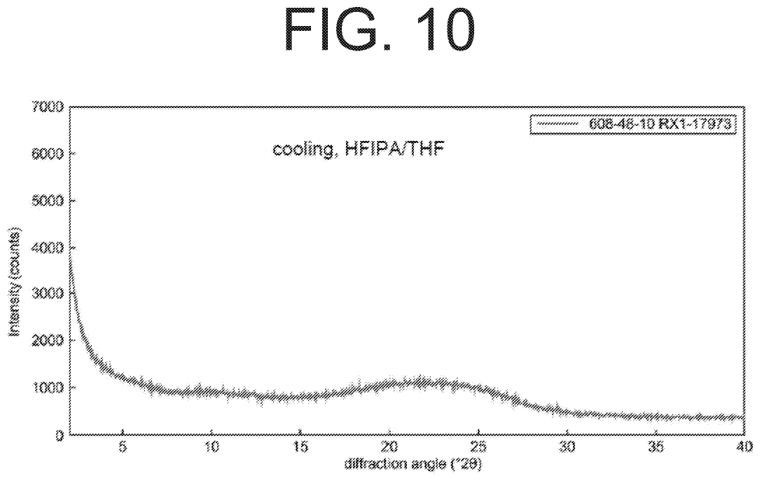

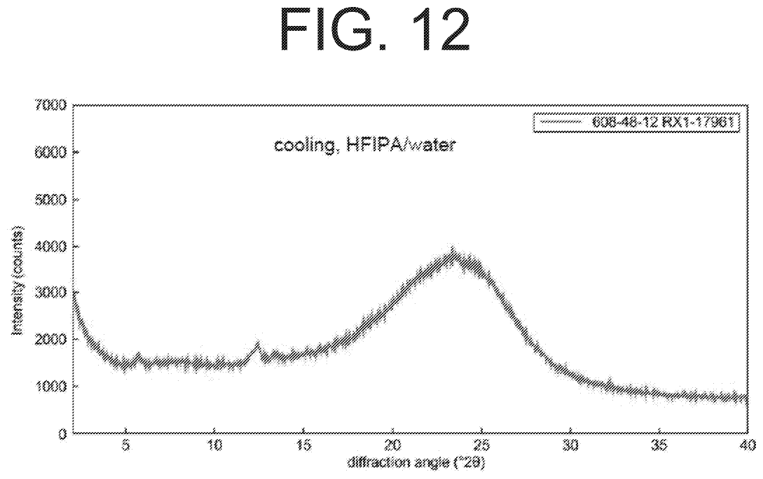

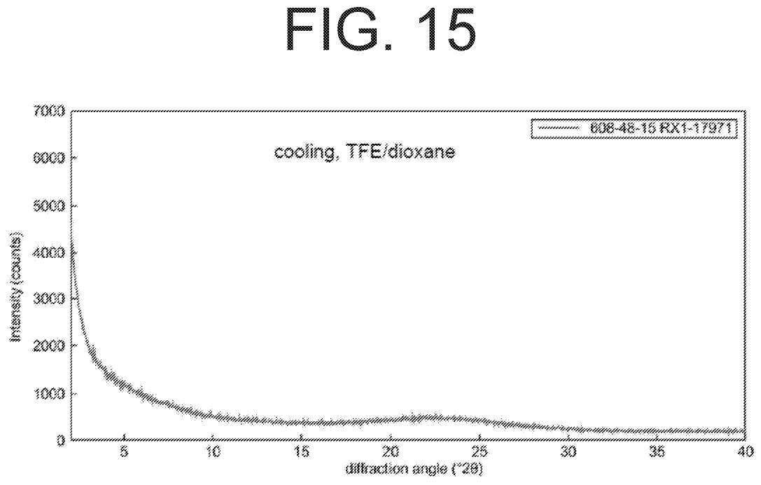

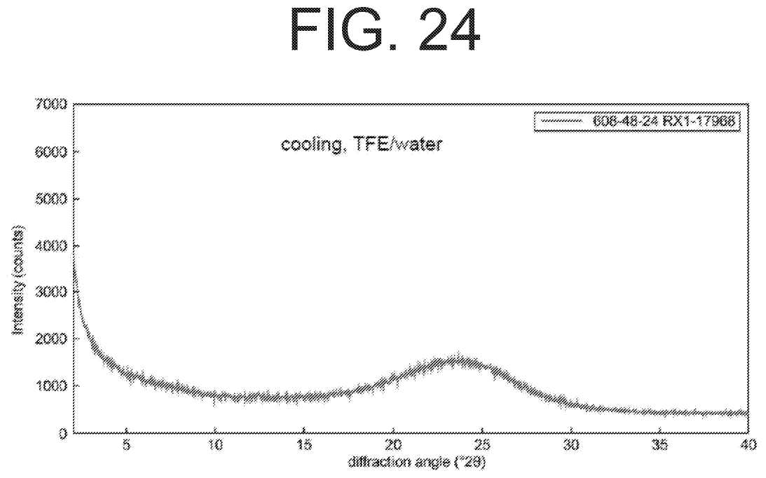

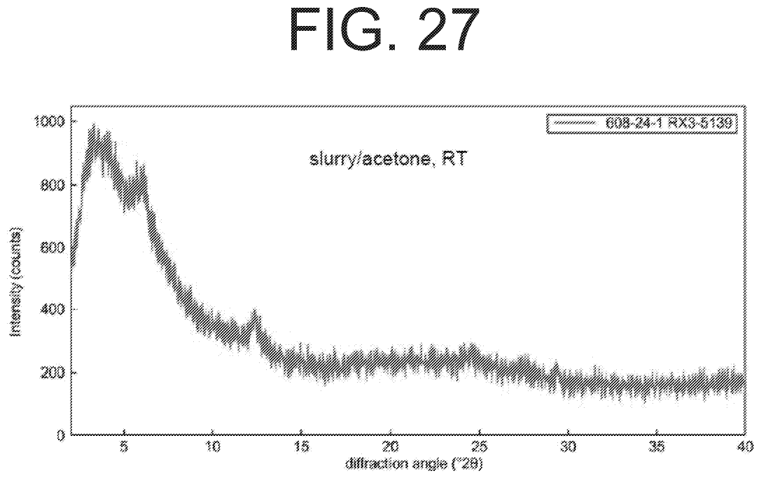

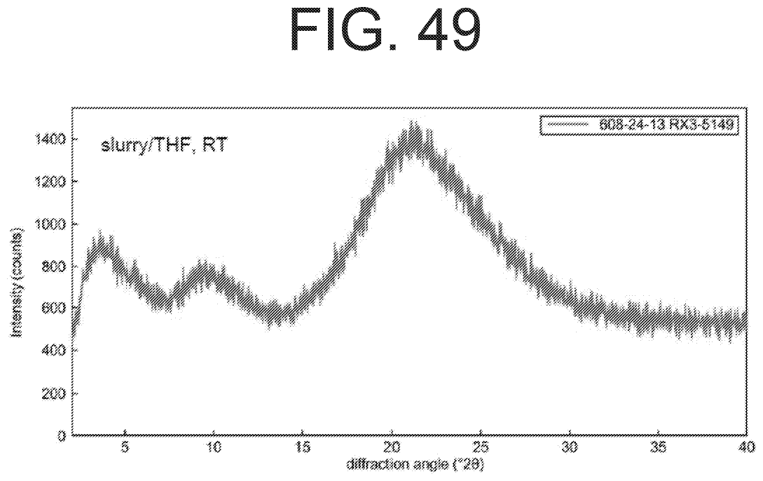

In some embodiments, the Compound of Formula (I) disclosed herein is in the form of a non-crystalline solid. In some embodiments, the non-crystalline solid form of the Compound of Formula (I) is substantially pure. In some embodiments, the purity of the non-crystalline solid form of the Compound of Formula (I) is determined by LC. In some embodiments, the non-crystalline solid form of Compound of Formula (I) is characterized by an x-ray powder diffractogram substantially as shown in FIG. 1, FIG. 3, FIG. 5, FIG. 8, FIG. 9, FIG. 10, FIG. 12, FIG. 15, FIG. 24, FIG. 27, and/or FIG. 49.

In some embodiments, a non-crystalline solid form of Compound of Formula (I) is produced by cooling a heated mixture of a solvent, an anti-solvent, and Compound of Formula (I). In some embodiments, the mixture is cooled from 60° C. to −15° C. In some embodiments, the solvent is 1,1,1,3,3,3-hexafluoro-2-propanol. In some embodiments, the solvent is 2,2,2-trifluorethanol. In some embodiments, the anti-solvent is chosen from acetone, dioxane, ethyl acetate, 2-methyltetrahydrofuran, 2-propanol, tetrahydrofuran, water, and a mixture thereof. In some embodiments, the solvent is 1,1,1,3,3,3-hexafluoro-2-propanol and the anti-solvent is acetone. In some embodiments, the solvent is 1,1,1,3,3,3-hexafluoro-2-propanol and the anti-solvent is dioxane. In some embodiments, the solvent is 1,1,1,3,3,3-hexafluoro-2-propanol and the anti-solvent is ethyl acetate. In some embodiments, the solvent is 1,1,1,3,3,3-hexafluoro-2-propanol and the anti-solvent is 2-methyltetrahydrofuran. In some embodiments, the solvent is 1,1,1,3,3,3-hexafluoro-2-propanol and the anti-solvent is 2-propanol. In some embodiments, the solvent is 1,1,1,3,3,3-hexafluoro-2-propanol and the anti-solvent is tetrahydrofuran. In some embodiments, the solvent is 1,1,1,3,3,3-hexafluoro-2-propanol and the anti-solvent is water. In some embodiments, the solvent is 2,2,2-trifluorethanol and the anti-solvent is dioxane. In some embodiments, the solvent is 2,2,2-trifluorethanol and the anti-solvent is water.

In some embodiments, a non-crystalline solid form of Compound of Formula (I) is produced by slurrying a mixture of at least one solvent and Compound of Formula (I). In some embodiments, the mixture is slurried at room temperature.

In some embodiments, the mixture is slurried for seven days. In some embodiments, the solvent is acetonitrile. In some embodiments, the at least one solvent is tetrahydrofuran.

In some embodiments, provided herein is a pharmaceutical composition comprising Compound of Formula (I) formed from combining non-crystalline solid form of Compound of Formula (I) and at least one additional component chosen from pharmaceutically acceptable carriers, pharmaceutically acceptable vehicles, and pharmaceutically acceptable excipients. In some embodiments, provided herein is a pharmaceutical composition consisting of Compound of Formula (I) formed from combining non-crystalline solid form of Compound of Formula (I) and at least one additional component chosen from pharmaceutically acceptable carriers, pharmaceutically acceptable vehicles, and pharmaceutically acceptable excipients. In some embodiments, provided herein is a pharmaceutical composition consisting essentially of Compound of Formula (I) formed from combining non-crystalline solid form of Compound of Formula (I) and at least one additional component chosen from pharmaceutically acceptable carriers, pharmaceutically acceptable vehicles, and pharmaceutically acceptable excipients.

Methods for Making Compound of Formula (I)

In some embodiments, provided herein is a method for making Compound of Formula (I) comprising slurrying Compound of Formula (I) in a solvent mixture comprising water and at least one first solvent chosen from acetonitrile, isopropanol, acetone, ethanol, and tetrahydrofuran. In some embodiments, the at least one first solvent is acetonitrile. In some embodiments, the at least one first solvent is ethanol. In some embodiments, the at least one first solvent is tetrahydrofuran.

In some embodiments, the ratio of the volume of the at least one first solvent to the volume of the water ranges from 99/1 to 1/99. In some embodiments, the ratio of the volume of the at least one first solvent to the volume of the water is 1/1. In some embodiments, the ratio of the volume of the at least one first solvent to the volume of the water is 95/5.

In some embodiments, the method further comprises combining a compound of Formula Int-3:

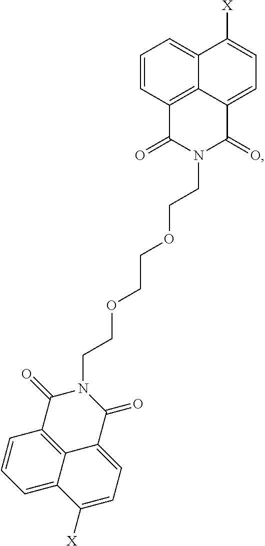

wherein X is chosen from F, Cl, Br, and I, and a compound of Formula Int-2:

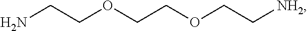

to form Compound of Formula (I). In some embodiments, the combining of the compound of Formula Int-3 and the compound of Formula Int-2 is carried out in benzene, xylene, acetonitrile, tetrahydrofuran, dimethylacetamide, dimethylformamide, N-methyl-2-pyrrolidone, ethanol, isopropanol, n-butanol, toluene, and/or pyridine. In some embodiments, the combining of the compound of Formula Int-3 and the compound of Formula Int-2 is carried out in toluene.

In some embodiments, the method further comprises combining a compound of Formula Int-1:

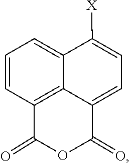

wherein X is chosen from F, Cl, Br, and I, and the compound of Formula Int-2 to form the compound of Formula Int-3. In some embodiments, the method is carried out in dimethylformamide, dimethylsulfoxide, N-methyl-2-pyrrolidone, dimethylacetamide, triethylamine and/or N,N-diisopropylethylamine.

In some embodiments, the compound of Formula Int-1 is recrystallized prior to the combining of the compound of Formula Int-1 and the compound of Formula Int-2.

In some embodiments, the method results in an increased yield relative to a method for making Compound of Formula (I) which does not comprise slurrying Compound of Formula (I) in a solvent mixture comprising at least one first solvent and water. In some embodiments, the method results in fewer process impurities in a composition of Compound of Formula (I) relative to a method for making Compound of Formula (I) which does not comprise slurrying Compound of Formula (I) in a solvent mixture comprising at least one first solvent and water.

In some embodiments, the method results in a lower number of process impurities in a composition comprising Compound of Formula (I) relative to a method for making Compound of Formula (I) which does not comprise slurrying Compound of Formula (I) in a solvent mixture comprising water and at least one first solvent chosen from acetonitrile, isopropanol, acetone, ethanol, and tetrahydrofuran. In some embodiments, the method results in at least one fewer process impurity, at least two fewer process impurities, at least three fewer process impurities, at least four fewer process impurities, at least five fewer process impurities, at least six fewer process impurities, at least seven fewer process impurities, at least eight fewer process impurities, at least nine fewer process impurities, at least ten fewer process impurities, at least eleven fewer process impurities, at least twelve fewer process impurities, at least thirteen fewer process impurities, at least fourteen fewer process impurities, or at least fifteen fewer process impurities. In some embodiments, the method results in one less process impurity, two fewer process impurities, three fewer process impurities, four fewer process impurities, five fewer process impurities, six fewer process impurities, seven fewer process impurities, eight fewer process impurities, nine fewer process impurities, ten fewer process impurities, eleven fewer process impurities, twelve fewer process impurities, thirteen fewer process impurities, fourteen fewer process impurities, or fifteen fewer process impurities relative to a method for making Compound of Formula (I) which does not comprise slurrying Compound of Formula (I) in a solvent mixture comprising water and at least one first solvent chosen from acetonitrile, isopropanol, acetone, ethanol, and tetrahydrofuran. In some embodiments, the method results in at least one fewer process impurity, at least two fewer process impurities, at least three fewer process impurities, at least four process impurities, or at least five fewer process impurities. In some embodiments, the method results in at least one fewer process impurity. In some embodiments, the method results in at least two fewer process impurities. In some embodiments, the method results in at least three fewer process impurities. In some embodiments, the method results in at least four fewer process impurities. In some embodiments, the method results in at least five fewer process impurities.

In some embodiments, the method results in a decrease in the concentration of process impurities in a composition comprising Compound of Formula (I) relative to a method for making Compound of Formula (I) which does not comprise slurrying Compound of Formula (I) in a solvent mixture comprising water and a first solvent chosen from acetonitrile, isopropanol, acetone, ethanol, and tetrahydrofuran. In some embodiments, the method results in a concentration of less than 25%, less than 22.5%, less than 20%, less than 17.5%, less than 15%, less than 14%, less than 13%, less than 12%, less than 11%, less than 10%, less than 9.5%, less than 9%, less than 8.5%, less than 8%, less than 7.5%, less than 7%, less than 6.5%, less than 6%, less than 5.5%, less than 5%, less than 4.75%, less than 4.5%, less than 4.25%, less than 4%, less than 3.75%, less than 3.5%, less than 3.25%, less than 3%, less than 2.9%, less than 2.8%, less than 2.7%, less than 2.6%, less than 2.5%, less than 2.4%, less than 2.3%, less than 2.2%, less than 2.1%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.75%, less than 0.7%, less than 0.65%, less than 0.6%, less than 0.55%, less than 0.5%, less than 0.45%, less than 0.4%, less than 0.35%, less than 0.3%, less than 0.25%, less than 0.2%, less than 0.15%, less than 0.1%, or less than 0.05% of process impurities. In some embodiments, the concentration of process impurities is determined by LC.

In some embodiments, the method results in a concentration of less than 15% of process impurities, as determined by LC. In some embodiments, the method results in a concentration of less than 12.5% of process impurities, as determined by LC. In some embodiments, the method results in a concentration of less than 10% of process impurities, as determined by LC. In some embodiments, the method results in a concentration of less than 7.5% of process impurities, as determined by LC. In some embodiments, the method results in a concentration of less than 5% of process impurities, as determined by LC. In some embodiments, the method results in a concentration of less than 4% of process impurities, as determined by LC. In some embodiments, the method results in a concentration of less than 3% of process impurities, as determined by LC. In some embodiments, the method results in a concentration of less than 2% of process impurities, as determined by LC. In some embodiments, the method results in a concentration of less than 1.5% of process impurities, as determined by LC. In some embodiments, the method results in a concentration of less than 1% of process impurities, as determined by LC. In some embodiments, the method results in a concentration of less than 0.75% of process impurities, as determined by LC. In some embodiments, the method results in a concentration of less than 0.5% of process impurities, as determined by LC. In some embodiments, the method results in a concentration of less than 0.35% of process impurities, as determined by LC. In some embodiments, the method results in a concentration of less than 0.25% of process impurities, as determined by LC. In some embodiments, the method results in a concentration of less than 0.15% of process impurities, as determined by LC. In some embodiments, the method results in a concentration of less than 0.1% of process impurities, as determined by LC. In some embodiments, the method results in a concentration of less than 0.05% of process impurities, as determined by LC.

Methods for Improving the Synthetic Yield of Compound of Formula (I)

In some embodiments, provided herein is a method for improving the synthetic yield of Compound of Formula (I) comprising slurrying Compound of Formula (I) in a solvent mixture comprising water and at least one first solvent chosen from acetonitrile, isopropanol, acetone, ethanol, and tetrahydrofuran. In some embodiments, the at least one first solvent is acetonitrile. In some embodiments, the at least one first solvent is ethanol. In some embodiments, the at least one first solvent is tetrahydrofuran.

In some embodiments, the ratio of the volume of the at least one first solvent to the volume of the water ranges from 99/1 to 1/99. In some embodiments, the ratio of the volume of the at least one first solvent to the volume of the water is 1/1. In some embodiments, the ratio of the volume of the at least one first solvent to the volume of the water is 95/5.

In some embodiments, the method further comprises combining a compound of Formula Int-3:

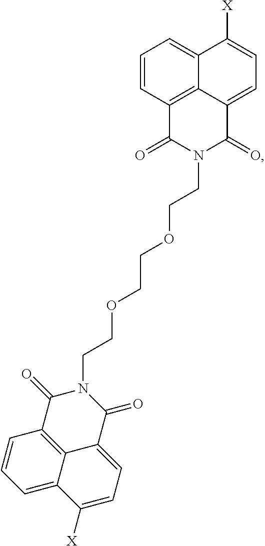

wherein X is chosen from F, Cl, Br, and I, and a compound of Formula Int-2:

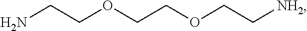

to form the Compound of Formula (I). In some embodiments, the combining of the compound of Formula Int-3 and the compound of Formula Int-2 is carried out in benzene, xylene, acetonitrile, tetrahydrofuran, dimethylacetamide, dimethylformamide, N-methyl-2-pyrrolidone, ethanol, isopropanol, n-butanol, toluene, and/or pyridine. In some embodiments, the combining of the compound of Formula Int-3 and the compound of Formula Int-2 is carried out in toluene.

In some embodiments, the method further comprises combining a compound of Formula Int-1:

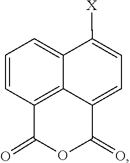

wherein X is chosen from F, Cl, Br, and I, and the compound of Formula Int-2 to form the compound of Formula Int-3. In some embodiments, the method is carried out in dimethylformamide, dimethylsulfoxide, N-methyl-2-pyrrolidone, dimethylacetamide triethylamine, and/or N,N-diisopropylethylamine.

In some embodiments, the compound of Formula Int-1 is recrystallized prior to combining the compound of Formula Int-1 and the compound of Formula Int-2.

In some embodiments, the method results in a lower number of process impurities in a composition comprising Compound of Formula (I) relative to a method for making the Compound of Formula (I) which does not comprise slurrying Compound of Formula (I) in a solvent mixture comprising water and at least one first solvent chosen from acetonitrile, isopropanol, acetone, ethanol, and tetrahydrofuran. In some embodiments, the method results in at least one fewer process impurity, at least two fewer process impurities, at least three fewer process impurities, at least four fewer process impurities, at least five fewer process impurities, at least six fewer process impurities, at least seven fewer process impurities, at least eight fewer process impurities, at least nine fewer process impurities, at least ten fewer process impurities, at least eleven fewer process impurities, at least twelve fewer process impurities, at least thirteen fewer process impurities, at least fourteen fewer process impurities, or at least fifteen fewer process impurities. In some embodiments, the method results in one less process impurity, two fewer process impurities, three fewer process impurities, four fewer process impurities, five fewer process impurities, six fewer process impurities, seven fewer process impurities, eight fewer process impurities, nine fewer process impurities, ten fewer process impurities, eleven fewer process impurities, twelve fewer process impurities, thirteen fewer process impurities, fourteen fewer process impurities, or fifteen fewer process impurities relative to a method for making Compound of Formula (I) which does not comprise slurrying Compound of Formula (I) in a solvent mixture comprising water and at least one first solvent chosen from acetonitrile, isopropanol, acetone, ethanol, and tetrahydrofuran. In some embodiments, the method results in at least one fewer process impurity, at least two fewer process impurities, at least three fewer process impurities, at least four process impurities, or at least five fewer process impurities. In some embodiments, the method results in at least one fewer process impurity. In some embodiments, the method results in at least two fewer process impurities. In some embodiments, the method results in at least three fewer process impurities. In some embodiments, the method results in at least four fewer process impurities. In some embodiments, the method results in at least five fewer process impurities.

In some embodiments, the method results in a decrease in the concentration of process impurities in a composition comprising Compound of Formula (I) relative to a method for making the Compound of Formula (I) which does not comprise slurrying the Compound of Formula (I) in a solvent mixture comprising water and at least one first solvent chosen from acetonitrile, isopropanol, acetone, ethanol, and tetrahydrofuran. In some embodiments, the method results in a concentration of less than 25%, less than 22.5%, less than 20%, less than 17.5%, less than 15%, less than 14%, less than 13%, less than 12%, less than 11%, less than 10%, less than 9.5%, less than 9%, less than 8.5%, less than 8%, less than 7.5%, less than 7%, less than 6.5%, less than 6%, less than 5.5%, less than 5%, less than 4.75%, less than 4.5%, less than 4.25%, less than 4%, less than 3.75%, less than 3.5%, less than 3.25%, less than 3%, less than 2.9%, less than 2.8%, less than 2.7%, less than 2.6%, less than 2.5%, less than 2.4%, less than 2.3%, less than 2.2%, less than 2.1%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.75%, less than 0.7%, less than 0.65%, less than 0.6%, less than 0.55%, less than 0.5%, less than 0.45%, less than 0.4%, less than 0.35%, less than 0.3%, less than 0.25%, less than 0.2%, less than 0.15%, less than 0.1%, or less than 0.05% of process impurities. In some embodiments, the concentration of process impurities is determined by LC.

In some embodiments, the method results in a concentration of less than 15% of process impurities, as determined by LC. In some embodiments, the method results in a concentration of less than 12.5% of process impurities, as determined by LC. In some embodiments, the method results in a concentration of less than 10% of process impurities, as determined by LC. In some embodiments, the method results in a concentration of less than 7.5% of process impurities, as determined by LC. In some embodiments, the method results in a concentration of less than 5% of process impurities, as determined by LC. In some embodiments, the method results in a concentration of less than 4% of process impurities, as determined by LC. In some embodiments, the method results in a concentration of less than 3% of process impurities, as determined by LC. In some embodiments, the method results in a concentration of less than 2% of process impurities, as determined by LC. In some embodiments, the method results in a concentration of less than 1.5% of process impurities, as determined by LC. In some embodiments, the method results in a concentration of less than 1% of process impurities, as determined by LC. In some embodiments, the method results in a concentration of less than 0.75% of process impurities, as determined by LC. In some embodiments, the method results in a concentration of less than 0.5% of process impurities, as determined by LC.

Methods of Reducing Process Impurities in a Composition Comprising a Compound of Formula (I)

In some embodiments, provided herein is a method of reducing process impurities in a composition comprising Compound of Formula (I) comprising slurrying Compound of Formula (I) in a solvent mixture comprising water and at least one first solvent chosen from acetonitrile, isopropanol, acetone, ethanol, and tetrahydrofuran. In some embodiments, the at least one first solvent is acetonitrile. In some embodiments, the at least one first solvent is ethanol. In some embodiments, the at least one first solvent is tetrahydrofuran.

In some embodiments, the ratio of the volume of the at least one first solvent to the volume of the water ranges from 99/1 to 1/99. In some embodiments, the ratio of the volume of the at least one first solvent to the volume of the water is 1/1. In some embodiments, the ratio of the volume of the at least one first solvent to the volume of the water is 95/5.

In some embodiments, the method further comprises combining a compound of Formula Int-3:

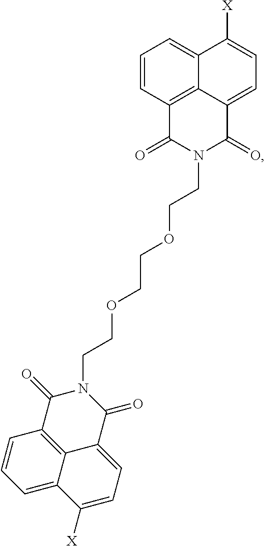

wherein X is chosen from F, Cl, Br, and I, and a compound of Formula Int-2:

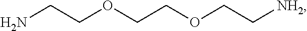

to form the Compound of Formula (I). In some embodiments, the combining of the compound of Formula Int-3 and the compound of Formula Int-2 is carried out in benzene, xylene, acetonitrile, tetrahydrofuran, dimethylacetamide, dimethylformamide, N-methyl-2-pyrrolidone, ethanol, isopropanol, n-butanol, toluene, and/or pyridine. In some embodiments, the combining of the compound of Formula Int-3 and the compound of Formula Int-2 is carried out in toluene.

In some embodiments, the method further comprises combining a compound of Formula Int-1:

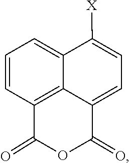

wherein X is chosen from F, Cl, Br, and I, and the compound of Formula Int-2 to form the compound of Formula Int-3. In some embodiments, the method is carried out in dimethylformamide, dimethylsulfoxide, N-methyl-2-pyrrolidone, dimethylacetamide, triethylamine, and/or N,N-diisopropylethylamine.

In some embodiments, the compound of Formula Int-1 is recrystallized prior to the combining of the compound of Formula Int-1 and the compound of Formula Int-2.

In some embodiments, the method results in a lower number of process impurities in a composition comprising Compound of Formula (I) relative to a method for making Compound of Formula (I) which does not comprise slurrying Compound of Formula (I) in a solvent mixture comprising water and a first solvent chosen from acetonitrile, isopropanol, acetone, ethanol, and tetrahydrofuran. In some embodiments, the method results in at least one fewer process impurity, at least two fewer process impurities, at least three fewer process impurities, at least four fewer process impurities, at least five fewer process impurities, at least six fewer process impurities, at least seven fewer process impurities, at least eight fewer process impurities, at least nine fewer process impurities, at least ten fewer process impurities, at least eleven fewer process impurities, at least twelve fewer process impurities, at least thirteen fewer process impurities, at least fourteen fewer process impurities, or at least fifteen fewer process impurities. In some embodiments, the method results in one less process impurity, two fewer process impurities, three fewer process impurities, four fewer process impurities, five fewer process impurities, six fewer process impurities, seven fewer process impurities, eight fewer process impurities, nine fewer process impurities, ten fewer process impurities, eleven fewer process impurities, twelve fewer process impurities, thirteen fewer process impurities, fourteen fewer process impurities, or fifteen fewer process impurities relative to a method for making Compound of Formula (I) which does not comprise slurrying Compound of Formula (I) in a solvent mixture comprising water and a first solvent chosen from acetonitrile, isopropanol, acetone, ethanol, and tetrahydrofuran. In some embodiments, the method results in at least one fewer process impurity, at least two fewer process impurities, at least three fewer process impurities, at least four fewer process impurities, or at least five fewer process impurities. In some embodiments, the method results in at least one fewer process impurity. In some embodiments, the method results in at least two fewer process impurities. In some embodiments, the method results in at least three fewer process impurities. In some embodiments, the method results in at least four fewer process impurities. In some embodiments, the method results in at least five fewer process impurities.

In some embodiments, the method results in a decrease in the concentration of process impurities in a composition comprising Compound of Formula (I) relative to a method for making Compound of Formula (I) which does not comprise slurrying Compound of Formula (I) in a solvent mixture comprising water and at least one first solvent chosen from acetonitrile, isopropanol, acetone, ethanol, and tetrahydrofuran. In some embodiments, the method results in a concentration of less than 25%, less than 22.5%, less than 20%, less than 17.5%, less than 15%, less than 14%, less than 13%, less than 12%, less than 11%, less than 10%, less than 9.5%, less than 9%, less than 8.5%, less than 8%, less than 7.5%, less than 7%, less than 6.5%, less than 6%, less than 5.5%, less than 5%, less than 4.75%, less than 4.5%, less than 4.25%, less than 4%, less than 3.75%, less than 3.5%, less than 3.25%, less than 3%, less than 2.9%, less than 2.8%, less than 2.7%, less than 2.6%, less than 2.5%, less than 2.4%, less than 2.3%, less than 2.2%, less than 2.1%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.75%, less than 0.7%, less than 0.65%, less than 0.6%, less than 0.55%, less than 0.5%, less than 0.45%, less than 0.4%, less than 0.35%, less than 0.3%, less than 0.25%, less than 0.2%, less than 0.15%, less than 0.1%, or less than 0.05% of process impurities. In some embodiments, the concentration of process impurities is determined by LC.

In some embodiments, the method results in a concentration of less than 15% of process impurities, as determined by LC. In some embodiments, the method results in a concentration of less than 12.5% of process impurities, as determined by LC. In some embodiments, the method results in a concentration of less than 10% of process impurities, as determined by LC. In some embodiments, the method results in a concentration of less than 7.5% of process impurities, as determined by LC. In some embodiments, the method results in a concentration of less than 5% of process impurities, as determined by LC. In some embodiments, the method results in a concentration of less than 4% of process impurities, as determined by LC. In some embodiments, the method results in a concentration of less than 3% of process impurities, as determined by LC. In some embodiments, the method results in a concentration of less than 2% of process impurities, as determined by LC. In some embodiments, the method results in a concentration of less than 1.5% of process impurities, as determined by LC. In some embodiments, the method results in a concentration of less than 1% of process impurities, as determined by LC. In some embodiments, the method results in a concentration of less than 0.75% of process impurities, as determined by LC. In some embodiments, the method results in a concentration of less than 0.5% of process impurities, as determined by LC. In some embodiments, the method results in a concentration of less than 0.35% of process impurities, as determined by LC. In some embodiments, the method results in a concentration of less than 0.25% of process impurities, as determined by LC. In some embodiments, the method results in a concentration of less than 0.15% of process impurities, as determined by LC. In some embodiments, the method results in a concentration of less than 0.1% of process impurities, as determined by LC. In some embodiments, the method results in a concentration of less than 0.05% of process impurities, as determined by LC.

Methods for Making Form 1 of Compound of Formula (I)

In some embodiments, provided herein is a method for making Form 1 of Compound of Formula (I) comprising isolating Form 1 from a mixture comprising Compound of Formula (I) and at least one solvent. In some embodiments, the at least one solvent is chosen from acetonitrile, chloroform, dichloromethane, 1,4-dioxane, dimethylformamide, dimethylsulfoxide, ethanol, ethyl acetate, diethyl ether, methanol, methylethylketone, 2-methyl-tetrahydrofuran, 2-propanol, tetrahydrofuran, toluene, and water. In some embodiments, the at least one solvent is chosen from acetonitrile, ethanol, methylethylketone, tetrahydrofuran, and water. In some embodiments, the at least one solvent comprises a first solvent and at least one second solvent. In some embodiments, the at least one first solvent is acetonitrile and the at least one second solvent is water.

In some embodiments, the ratio of the volume of the at least one first solvent to the volume of the at least one second solvent ranges from 99/1 to 1/99. In some embodiments, the ratio of the volume of the at least one first solvent to the volume of the at least one second solvent ranges from 50/1 to 1/50. In some embodiments, the ratio of the volume of the at least one first solvent to the volume of the at least one second solvent ranges from 25/1 to 1/25. In some embodiments, the ratio of the volume of the at least one first solvent to the volume of the at least one second solvent ranges from 15/1 to 1/15. In some embodiments, the ratio of the volume of the at least one first solvent to the volume of the at least one second solvent ranges from 10/1 to 1/10. In some embodiments, the ratio of the volume of the at least one first solvent to the volume of the at least one second solvent ranges from 5/1 to 1/5. In some embodiments, the ratio of the volume of the at least one first solvent to the volume of the at least one second solvent is 1/1.

Methods for Making Form 2 of Compound of Formula (I)

In some embodiments, provided herein is a method for making Form 2 of Compound of Formula (I) comprising isolating Form 2 from a mixture of Compound of Formula (I) and at least one first solvent. In some embodiments, the mixture further comprises at least one second solvent. In some embodiments, the at least one first solvent is acetone. In some embodiments, the at least one second solvent is water. In some embodiments, the at least one first solvent is acetone and the at least one second solvent is water.

In some embodiments, the ratio of the volume of the at least one first solvent to the volume of the at least one second solvent ranges from 99/1 to 1/99. In some embodiments, the ratio of the volume of the at least one first solvent to the volume of the at least one second solvent is 95/5.

Methods for Making Diacetate Salt of Compound of Formula (I)

In some embodiments, provided herein is a method for making a diacetate salt of Compound of Formula (I).

In some embodiments, the method comprises recrystallizing a compound of Formula Int-1:

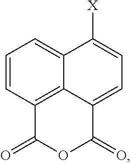

wherein X is chosen from F, Cl, Br, and I, from a solvent e.g., dimethylacetamide.

In some embodiments, the method further comprises combining an optionally recrystallized compound of Formula Int-1:

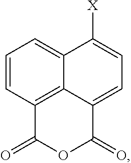

wherein X is chosen from F, Cl, Br, and I, and a compound of Formula Int-2:

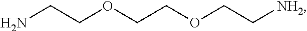

to form a compound of Formula Int-3:

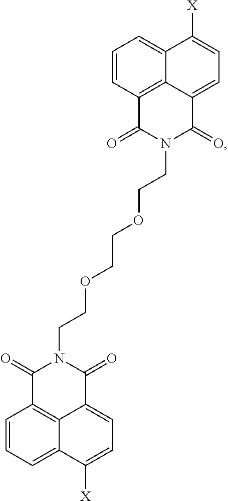

wherein X is chosen from F, Cl, Br, and I. In some embodiments, the combining of the optionally recrystallized compound of Formula Int-1 and the compound of Formula Int-2 is carried out in dimethylformamide, dimethylsulfoxide, N-methyl-2-pyrrolidone, dimethylacetamide, triethylamine and/or N,N-diisopropylethylamine.

In some embodiments, the method further comprises combining the compound of Formula Int-3:

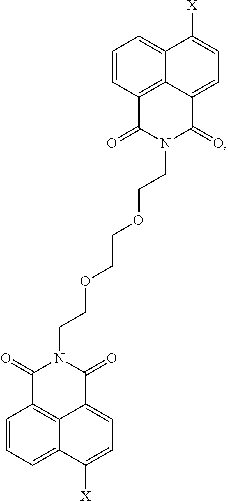

wherein X is chosen from F, Cl, Br, and I, and a compound of Formula Int-2:

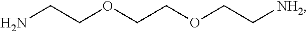

to form Compound of Formula (I):

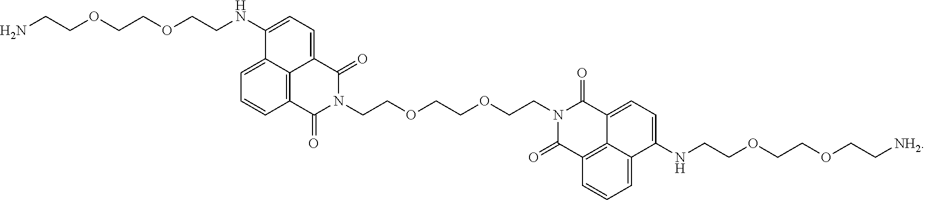 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

In some embodiments, the combining of the compound of Formula Int-3 and the compound of Formula Int-2 is carried out in benzene, xylene, acetonitrile, tetrahydrofuran, dimethylacetamide, dimethylformamide, N-methyl-2-pyrrolidone, ethanol, isopropanol, n-butanol, toluene, and/or pyridine. In some embodiments, the combining of the compound of Formula Int-3 and the compound of Formula Int-2 is carried out in toluene.

In some embodiments, the method further comprises slurrying a Compound of Formula (I) in a solvent mixture comprising water and at least one first solvent chosen from acetonitrile, isopropanol, acetone, ethanol, and tetrahydrofuran. In some embodiments, the at least one first solvent is acetonitrile. In some embodiments, the at least one first solvent is ethanol. In some embodiments, the at least one first solvent is tetrahydrofuran. In some embodiments, the ratio of the volume of the at least one first solvent to the volume of the water ranges from 99/1 to 1/99. In some embodiments, the ratio of the volume of the at least one first solvent to the volume of the water is 1/1. In some embodiments, the ratio of the volume of the at least one first solvent to the volume of the water is 95/5.

In some embodiments, the method further comprises combining a Compound of Formula (I) with at least one acid. In some embodiments, the at least one acid is chosen from hydrochloric acid, hydrobromic acid, sulfuric acid, phosphoric acid, nitric acid, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, and polygalacturonic acid. In some embodiments, the at least one acid is acetic acid.

In some embodiments, the method further comprises combining a diacetate salt of Compound of Formula (I), activated carbon, and at least one solvent. In some embodiments, the at least one solvent is chosen from acetonitrile, ethanol, tetrahydrofuran, and water. In some embodiments, the at least one solvent comprises water and acetonitrile. In some embodiments, the ratio of the volume of acetonitrile to the volume of water ranges from 99/1 to 1/99. In some embodiments, the ratio of the volume of acetonitrile to the volume of water ranges from 75/1 to 1/75. In some embodiments, the ratio of the volume of acetonitrile to the volume of water ranges from 50/1 to 1/50. In some embodiments, the ratio of the volume of acetonitrile to the volume of water ranges from 25/1 to 1/25. In some embodiments, the ratio of the volume of acetonitrile to the volume of water ranges from 15/1 to 1/15. In some embodiments, the ratio of the volume of acetonitrile to the volume of water ranges from 10/1 to 1/10. In some embodiments, the ratio of the volume of acetonitrile to the volume of water ranges from 5/1 to 1/5. In some embodiments, the ratio of the volume of acetonitrile to the volume of water is 1/1.

In some embodiments, the method further comprises isolating a diacetate salt of Compound of Formula (I) from a mixture comprising a diacetate salt of Compound of Formula (I), activated carbon, and at least one solvent. In some embodiments, the isolating comprises filtering the mixture through at least one filtration aid. In some embodiments, the at least one filtration aid is chosen from diatomaceous earth and at least one membrane filter. In some embodiments, the at least one membrane filter has a thickness ranging from 0.1 μm to 1 μm. In some embodiments, the at least one membrane filter has a thickness of 0.1 μm, 0.2 μm, 0.3 μm, 0.4 μm, 0.5 μm, 0.6 μm, 0.7 μm, 0.8 μm, 0.9 μm, or 1 μm. In some embodiments, the at least one membrane filter is 0.2 μm thick.

In some embodiments, provided herein is a method for making a diacetate salt of Compound of Formula (I) comprising isolating the diacetate salt of Compound of Formula (I) from a mixture comprising the diacetate salt of Compound of Formula (I), activated carbon, and at least one solvent. In some embodiments, the at least one solvent is chosen from acetonitrile, ethanol, tetrahydrofuran, and water. In some embodiments, the at least one solvent comprises water and acetonitrile. In some embodiments, the ratio of the volume of acetonitrile to the volume of water ranges from 99/1 to 1/99. In some embodiments, the ratio of the volume of acetonitrile to the volume of water ranges from 75/1 to 1/75. In some embodiments, the ratio of the volume of acetonitrile to the volume of water ranges from 50/1 to 1/50. In some embodiments, the ratio of the volume of acetonitrile to the volume of water ranges from 25/1 to 1/25. In some embodiments, the ratio of the volume of acetonitrile to the volume of water ranges from 15/1 to 1/15. In some embodiments, the ratio of the volume of acetonitrile to the volume of water ranges from 10/1 to 1/10. In some embodiments, the ratio of the volume of acetonitrile to the volume of water ranges from 5/1 to 1/5. In some embodiments, the ratio of the volume of acetonitrile to the volume of water is 1/1.

In some embodiments, the isolating comprises filtering the mixture through at least one filtration aid. In some embodiments, the at least one filtration aid is chosen from diatomaceous earth and at least one membrane filter. In some embodiments, the at least one membrane filter has a thickness ranging from 0.1 μm to 1 μm. In some embodiments, the at least one membrane filter has a thickness of 0.1 μm, 0.2 μm, 0.3 μm, 0.4 μm, 0.5 μm, 0.6 μm, 0.7 μm, 0.8 μm, 0.9 μm, or 1 μm. In some embodiments, the membrane filter is 0.2 μm thick.

In some embodiments, the method further comprises combining a Compound of Formula (I) with at least one acid. In some embodiments, the at least one acid is chosen from hydrochloric acid, hydrobromic acid, sulfuric acid, phosphoric acid, nitric acid, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, and polygalacturonic acid. In some embodiments, the at least one acid is acetic acid.

In some embodiments, the method further comprises slurrying Compound of Formula (I) in a solvent mixture comprising water and at least one first solvent chosen from acetonitrile, isopropanol, acetone, ethanol, and tetrahydrofuran. In some embodiments, the at least one first solvent is acetonitrile. In some embodiments, the at least one first solvent is ethanol. In some embodiments, the at least one first solvent is tetrahydrofuran.

In some embodiments, the ratio of the volume of the at least one first solvent to the volume of the water ranges from 99/1 to 1/99. In some embodiments, the ratio of the volume of the at least one first solvent to the volume of the water is 1/1. In some embodiments, the ratio of the volume of the at least one first solvent to the volume of the water is 95/5.

In some embodiments, the method further comprises combining a compound of Formula Int-3:

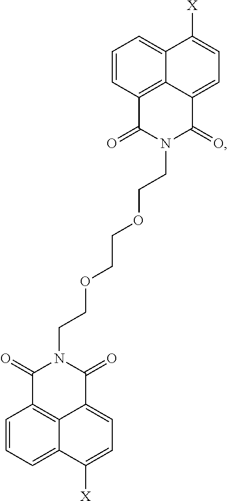

wherein X is chosen from F, Cl, Br, and I, and a compound of Formula Int-2:

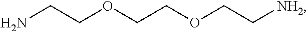

to form the Compound of Formula (I). In some embodiments, the combining of the compound of Formula Int-3 and the compound of Formula Int-2 is carried out in benzene, xylene, acetonitrile, tetrahydrofuran, dimethylacetamide, dimethylformamide, N-methyl-2-pyrrolidone, ethanol, isopropanol, n-butanol, toluene, and/or pyridine. In some embodiments, the combining of the compound of Formula Int-3 and the compound of Formula Int-2 is carried out in toluene.

In some embodiments, the method further comprises combining a compound of Formula Int-1:

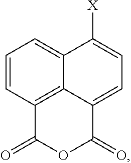

wherein X is chosen from F, Cl, Br, and I, and the compound of Formula Int-2 to form the compound of Formula Int-3. In some embodiments, the combining of the compound of Formula Int-1 and the compound of Formula Int-2 is carried out in dimethylformamide, dimethylsulfoxide, N-methyl-2-pyrrolidone, dimethylacetamide, triethylamine, and/or N,N-diisopropylethylamine.

In some embodiments, the compound of Formula Int-1 is recrystallized prior to carrying out the combining of the compound of Formula Int-1 and the compound of Formula Int-3.

In some embodiments, the method results in an increased yield relative to a method for making the diacetate salt of Compound of Formula (I) which does not comprise slurrying Compound of Formula (I) in a solvent mixture comprising water and at least one first solvent chosen from acetonitrile, isopropanol, acetone, ethanol, and tetrahydrofuran and/or isolating Diacetate Salt of Compound of Formula (I) from a mixture comprising Diacetate Salt of Compound of Formula (I), activated carbon, and at least one solvent. In some embodiments, the method results in fewer types of process impurities in a composition comprising the diacetate salt of Compound of Formula (I) which does not comprise slurrying Compound of Formula (I) in a solvent mixture comprising water and at least one first solvent chosen from acetonitrile, isopropanol, acetone, ethanol, and tetrahydrofuran and/or isolating Diacetate Salt of Compound of Formula (I) from a mixture comprising Diacetate Salt of Compound of Formula (I), activated carbon, and at least one solvent.

In some embodiments, the method results in a lower number of process impurities in a composition comprising Diacetate Salt of Compound of Formula (I) relative to a method for making Diacetate Salt of Compound of Formula (I) which does not comprise isolating Diacetate Salt of Compound of Formula (I) from a mixture comprising Diacetate Salt of Compound of Formula (I), activated carbon, and at least one solvent. In some embodiments, the method results in at least one fewer process impurity, at least two fewer process impurities, at least three fewer process impurities, at least four fewer process impurities, at least five fewer process impurities, at least six fewer process impurities, at least seven fewer process impurities, at least eight fewer process impurities, at least nine fewer process impurities, at least ten fewer process impurities, at least eleven fewer process impurities, at least twelve fewer process impurities, at least thirteen fewer process impurities, at least fourteen fewer process impurities, or at least fifteen fewer process impurities. In some embodiments, the method results in one less process impurity, two fewer process impurities, three fewer process impurities, four fewer process impurities, five fewer process impurities, six fewer process impurities, seven fewer process impurities, eight fewer process impurities, nine fewer process impurities, ten fewer process impurities, eleven fewer process impurities, twelve fewer process impurities, thirteen fewer process impurities, fourteen fewer process impurities, or fifteen fewer process impurities relative to a method for making Compound of Formula (I) which does not comprise slurrying Compound of Formula (I) in a solvent mixture comprising water and a first solvent chosen from acetonitrile, isopropanol, acetone, ethanol, and tetrahydrofuran. In some embodiments, the method results in at least one fewer process impurity, at least two fewer process impurities, at least three fewer process impurities, at least four process impurities, or at least five fewer process impurities. In some embodiments, the method results in at least one fewer process impurity. In some embodiments, the method results in at least two fewer process impurities. In some embodiments, the method results in at least three fewer process impurities. In some embodiments, the method results in at least four fewer process impurities. In some embodiments, the method results in at least five fewer process impurities.

In some embodiments, the method results in a decrease in the concentration of process impurities in a composition comprising Diacetate Salt of Compound of Formula (I) relative to a method for making Diacetate Salt of Compound of Formula (I) which does not comprise isolating Diacetate Salt of Compound of Formula (I) from a mixture comprising Diacetate Salt of Compound of Formula (I), activated carbon, and at least one solvent. In some embodiments, the method results in a concentration of less than 25%, less than 22.5%, less than 20%, less than 17.5%, less than 15%, less than 14%, less than 13%, less than 12%, less than 11%, less than 10%, less than 9.5%, less than 9%, less than 8.5%, less than 8%, less than 7.5%, less than 7%, less than 6.5%, less than 6%, less than 5.5%, less than 5%, less than 4.75%, less than 4.5%, less than 4.25%, less than 4%, less than 3.75%, less than 3.5%, less than 3.25%, less than 3%, less than 2.9%, less than 2.8%, less than 2.7%, less than 2.6%, less than 2.5%, less than 2.4%, less than 2.3%, less than 2.2%, less than 2.1%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.75%, less than 0.7%, less than 0.65%, less than 0.6%, less than 0.55%, less than 0.5%, less than 0.45%, less than 0.4%, less than 0.35%, less than 0.3%, less than 0.25%, less than 0.2%, less than 0.15%, less than 0.1%, or less than 0.05% of process impurities. In some embodiments, the concentration of process impurities is determined by LC.

In some embodiments, the method results in a concentration of less than 15% of process impurities, as determined by LC. In some embodiments, the method results in a concentration of less than 12.5% of process impurities, as determined by LC. In some embodiments, the method results in a concentration of less than 10% of process impurities, as determined by LC. In some embodiments, the method results in a concentration of less than 7.5% of process impurities, as determined by LC. In some embodiments, the method results in a concentration of less than 5% of process impurities, as determined by LC. In some embodiments, the method results in a concentration of less than 4% of process impurities, as determined by LC. In some embodiments, the method results in a concentration of less than 3% of process impurities, as determined by LC. In some embodiments, the method results in a concentration of less than 2% of process impurities, as determined by LC. In some embodiments, the method results in a concentration of less than 1.5% of process impurities, as determined by LC. In some embodiments, the method results in a concentration of less than 1% of process impurities, as determined by LC. In some embodiments, the method results in a concentration of less than 0.75% of process impurities, as determined by LC. In some embodiments, the method results in a concentration of less than 0.5% of process impurities, as determined by LC. In some embodiments, the method results in a concentration of less than 0.35% of process impurities, as determined by LC. In some embodiments, the method results in a concentration of less than 0.25% of process impurities, as determined by LC. In some embodiments, the method results in a concentration of less than 0.15% of process impurities, as determined by LC. In some embodiments, the method results in a concentration of less than 0.1% of process impurities, as determined by LC. In some embodiments, the method results in a concentration of less than 0.05% of process impurities, as determined by LC.

In some embodiments, provided herein is a pharmaceutical composition comprising a diacetate salt of Compound of Formula (I) produced according to any of the methods described herein and at least one additional component chosen from pharmaceutically acceptable carriers, pharmaceutically acceptable vehicles, and pharmaceutically acceptable excipients. In some embodiments, provided herein is a pharmaceutical composition consisting of a diacetate salt of Compound of Formula (I) produced according to any of the methods described herein and at least one additional component chosen from pharmaceutically acceptable carriers, pharmaceutically acceptable vehicles, and pharmaceutically acceptable excipients. In some embodiments, provided herein is a pharmaceutical composition consisting essentially of a diacetate salt of Compound of Formula (I) produced according to any of the methods described herein and at least one additional component chosen from pharmaceutically acceptable carriers, pharmaceutically acceptable vehicles, and pharmaceutically acceptable excipients.

### ADDITIONAL EMBODIMENTS

Embodiment 1. A method for making Form 1 of 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione) (“Compound of Formula (I)”) comprising isolating Form 1 from a mixture comprising Compound of Formula (I) and at least one solvent.

Embodiment 2. The method according to embodiment 1, wherein the at least one solvent is chosen from acetonitrile, chloroform, dichloromethane, 1,4-dioxane, dimethylformamide, dimethylsulfoxide, ethanol, ethyl acetate, diethyl ether, methanol, methylethylketone, 2-methyl-tetrahydrofuran, 2-propanol, tetrahydrofuran, toluene, and water.

Embodiment 3. The method according to embodiment 2, wherein the at least one solvent is chosen from acetonitrile, ethanol, methylethylketone, tetrahydrofuran, and water.

Embodiment 4. The method according to any one of embodiments 1 to 3, wherein the at least one solvent comprises a first solvent and a second solvent.

Embodiment 5. The method according to any of embodiments 2 to 4, wherein the at least one first solvent is acetonitrile and the at least one second solvent is water.

Embodiment 6. The method according to embodiment 4 or 5, wherein the ratio of the volume of the at least one first solvent to the volume of the at least one second solvent ranges from 99/1 to 1/99.

Embodiment 7. The method according to embodiment 6, wherein the ratio of the amount of the at least one first solvent to the amount of the at least one second solvent ranges from 5/1 to 1/5.

Embodiment 8. The method according to embodiment 7, wherein the ratio of the amount of the at least one first solvent to the amount of the at least one second solvent is 1/1.

Embodiment 9. A method for making a Compound of Formula (I) comprising:

slurrying Compound of Formula (I) in a solvent mixture comprising at least one first solvent chosen from acetonitrile, isopropanol, acetone, ethanol, and tetrahydrofuran; and water.

Embodiment 10. The method according to embodiment 9, wherein the at least one first solvent is acetonitrile.

Embodiment 11. The method according to embodiment 9, further comprising:

combining a compound of Formula Int-3:

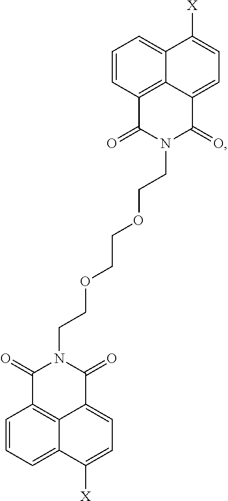

wherein X is chosen from F, Cl, Br, and I, and a compound of Formula Int-2:

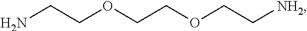

to form the Compound of Formula (I).

Embodiment 12. The method according to embodiment 11, wherein the combining of the compound of Formula Int-3 and the compound of Formula Int-2 is carried out in toluene.

Embodiment 13. The method according to embodiment 11, further comprising:

combining a compound of Formula Int-1:

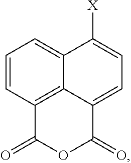

and the compound of Formula Int-2 to form the compound of Formula Int-3.

Embodiment 14. The method according to embodiment 13, wherein the combining of the compound of Formula Int-1 and the compound of Formula Int-2 is carried out in dimethylformamide, dimethylsulfoxide, N-methyl-2-pyrrolidone, dimethylacetamide, triethylamine, and/or N,N-diisopropylethylamine.

Embodiment 15. The method according to embodiment 13, wherein the compound of Formula Int-1 is recrystallized prior to combining with a compound of Formula Int-2.

Embodiment 16. The method according to any one of embodiments 9 to 15, wherein the ratio of acetonitrile to water is about 99/1 to about 1/99.

Embodiment 17. The method according to any one of embodiments 9 to 16, wherein the ratio of acetonitrile to water is about 1/1.

Embodiment 18. The method according to any one of embodiments 9 to 17, wherein the method results in an increased synthetic yield relative to a method for making Compound of Formula (I) which does not comprise slurrying Compound of Formula (I) in a solvent mixture comprising a first solvent and water.

Embodiment 19. The method according to any one of embodiments 9 to 17, wherein the method results in fewer process impurities in a composition of Compound of Formula (I) relative to a method for making Compound of Formula (I) which does not comprise slurrying Compound of Formula (I) in a solvent mixture comprising a first solvent and water.

Embodiment 20. The method according to embodiment 19, wherein the method results in a decrease in the concentration of process impurities.

Embodiment 21. The method according to embodiment 19, wherein the method results in a concentration of less than 1% of process impurities, as determined by LC.

Embodiment 22. The method according to embodiment 19, wherein the method results in a lower number of process impurities.

Embodiment 23. The method according to embodiment 19, wherein the method results in at least three fewer process impurities.

Embodiment 24. A method of improving the synthetic yield of Compound of Formula (I) comprising:

slurrying Compound of Formula (I) in a solvent mixture comprising a first solvent chosen from acetonitrile, isopropanol, acetone, ethanol, and tetrahydrofuran; and water.

Embodiment 25. The method according to embodiment 24, further comprising:

combining a compound of Formula Int-3:

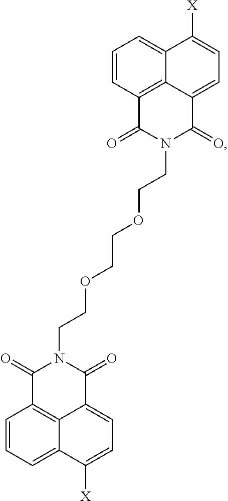

wherein X is chosen from F, Cl, Br, and I, and a compound of Formula Int-2:

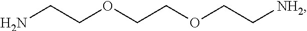

to form the Compound of Formula (I).

Embodiment 26. The method according to embodiment 25, wherein the combining of the compound of Formula Int-3 and the compound of Formula Int-2 is carried out in toluene.

Embodiment 27. The method according to embodiment 25, further comprising:

combining a compound of Formula Int-1:

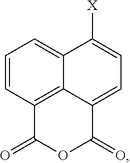

wherein X is chosen from F, Cl, Br, and I, and the compound of Formula Int-2 to form the compound of Formula Int-3.

Embodiment 28. The method according to embodiment 27, wherein the combining of the compound of Formula Int-1 and the compound of Formula Int-2 is carried out in dimethylformamide, dimethylsulfoxide, N-methyl-2-pyrrolidone, dimethylacetamide, triethylamine, and/or N,N-diisopropylethylamine.

Embodiment 29. The method according to embodiment 27, wherein the compound of Formula Int-1 is recrystallized prior to combining with the compound of Formula Int-2.

Embodiment 30. The method according to any one of embodiments 24 to 29, wherein the method results in an increased synthetic yield relative to a method for making Compound of Formula (I) which does not comprise slurrying Compound of Formula (I) in a solvent mixture comprising at least one first solvent chosen from acetonitrile, isopropanol, acetone, ethanol, and tetrahydrofuran; and water.

Embodiment 31. The method according to any one of embodiments 24 to 29, wherein the method results in fewer process impurities in a composition of Compound of Formula (I) relative to a method for making Compound of Formula (I) which does not comprise slurrying Compound of Formula (I) in a solvent mixture comprising at least one first solvent chosen from acetonitrile, isopropanol, acetone, ethanol, and tetrahydrofuran; and water.

Embodiment 32. The method according to embodiment 31, wherein the method results in a decrease in concentration of process impurities.

Embodiment 33. The method according to embodiment 31, wherein the method results in a concentration of less than 1% of process impurities, as determined by LC.

Embodiment 34. The method according to embodiment 31, wherein the method results in a lower number of process impurities.

Embodiment 35. The method according to embodiment 31, wherein the method results in at least three fewer process impurities.

Embodiment 36. A method of reducing process impurities in a composition comprising Compound of Formula (I), comprising:

slurrying Compound of Formula (I) in a solvent mixture comprising at least one first solvent chosen from acetonitrile, ethanol, methylethylketone, and tetrahydrofuran; and water.

Embodiment 37. The method according to embodiment 36, further comprising:

combining a compound of Formula Int-3:

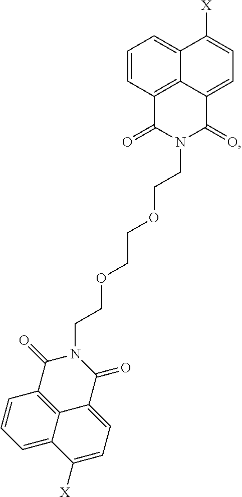

wherein X is chosen from F, Cl, Br, and I, and a compound of Formula Int-2:

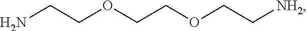

to form Compound of Formula (I).

Embodiment 38. The method according to embodiment 37, wherein the combining of the compound of Formula Int-3 and the compound of Formula Int-2 is carried out in toluene.

Embodiment 39. The method according to embodiment 37, further comprising:

combining a compound of Formula Int-1:

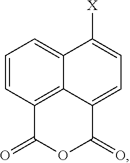

wherein X is chosen from F, Cl, Br, and I, and the compound of Formula Int-2 to form the compound of Formula Int-3.

Embodiment 40. The method according to embodiment 39, wherein the combining of the compound of Formula Int-1 and the compound of Formula Int-2 is carried out in dimethylformamide, dimethylsulfoxide, N-methyl-2-pyrrolidone, dimethylacetamide, triethylamine, and/or N,N-diisopropylethylamine.

Embodiment 41. The method according to embodiment 39, wherein the compound of Formula Int-1 is recrystallized prior to combining with the compound of Formula Int-2.

Embodiment 42. The method according to any one of embodiments 36 to 41, wherein the method results in an increased synthetic yield relative to a method for making Compound of Formula (I) which does not comprise slurrying Compound of Formula (I) in a solvent mixture comprising at least one first solvent chosen from acetonitrile, ethanol, methylethylketone, and tetrahydrofuran; and water.

Embodiment 43. The method according to any one of embodiments 36 to 41, wherein the method results in fewer process impurities in a composition of Compound of Formula (I) relative to a method for making Compound of Formula (I) which does not comprise slurrying Compound of Formula (I) in a solvent mixture comprising a first solvent chosen from acetonitrile, ethanol, methylethylketone, and tetrahydrofuran; and water.

Embodiment 44. The method according to embodiment 43, wherein the method results in a decrease in the concentration of process impurities.

Embodiment 45. The method according to embodiment 43, wherein the method results in a concentration of less than 1% of process impurities, as determined by LC.

Embodiment 46. The method according to embodiment 43, wherein the method results in a lower number of process impurities.

Embodiment 47. The method according to embodiment 43, wherein the method results in three fewer process impurities.

Embodiment 48. A method for making a diacetate salt of Compound of Formula (I) comprising:

isolating the diacetate salt of Compound of Formula (I) from a mixture comprising the diacetate salt of Compound of Formula (I), activated carbon, and at least one solvent.

Embodiment 49. The method according to embodiment 48, wherein the at least one solvent is chosen from acetonitrile, ethanol, tetrahydrofuran, and water.

Embodiment 50. The method according to embodiment 48, wherein the at least one solvent comprises water and acetonitrile.

Embodiment 51. The method according to embodiment 50, wherein the ratio of the volume of acetonitrile to the volume of water ranges from 99/1 to 1/99.

Embodiment 52. The method according to embodiment 51, wherein the ratio of the volume of acetonitrile to the volume of water is 1/1.

Embodiment 53. The method according to embodiment 48, wherein the isolating of the diacetate salt of Compound of Formula (I) comprises filtering the mixture through at least one filtration aid.

Embodiment 54. The method according to embodiment 53, wherein the at least one filtration aid is chosen from diatomaceous earth and at least one membrane filter.

Embodiment 55. The method according to embodiment 48, further comprising:

combining Compound of Formula (I) with at least one acid.

Embodiment 56. The method according to embodiment 55, wherein the at least one acid is chosen from hydrochloric acid, hydrobromic acid, sulfuric acid, phosphoric acid, nitric acid, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, and polygalacturonic acid.

Embodiment 57. The method according to embodiment 55 or 56, wherein the at least one acid is acetic acid.

Embodiment 58. The method according to embodiment 48, further comprising:

slurrying Compound of Formula (I) in a solvent mixture comprising at least one first solvent chosen from acetonitrile, isopropanol, acetone, ethanol, and tetrahydrofuran; and water.

Embodiment 59. The method according to embodiment 58, wherein the first solvent is acetonitrile.

Embodiment 60. The method according to embodiment 58 or 59, wherein the ratio of the volume of acetonitrile to the volume of water ranges from 10/1 to 1/10.

Embodiment 61. The method according to embodiment 58 or 59, wherein the ratio of the volume of acetonitrile to the volume of water is 1/1.

Embodiment 62. The method according to embodiment 48, further comprising:

combining a compound of Formula Int-3:

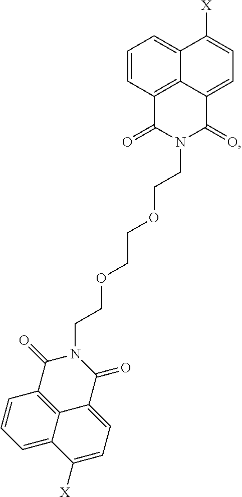

wherein X is chosen from F, Cl, Br, and I, and a compound of Formula Int-2:

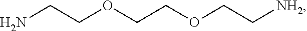

to form a Compound of Formula (I).

Embodiment 63. The method according to embodiment 62, wherein the combining of the compound of Formula Int-3 and the compound of Formula Int-2 is carried out in toluene.

Embodiment 64. The method according to embodiment 58, further comprising:

combining a compound of Formula Int-1:

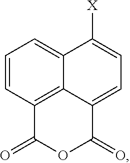

wherein X is chosen from F, Cl, Br, and I, and the compound of Formula Int-2 to form the compound of Formula Int-3.

Embodiment 65. The method according to embodiment 64, wherein the combining of the compound of Formula Int-1 and the compound of Formula Int-2 is carried out in dimethylformamide, dimethylsulfoxide, N-methyl-2-pyrrolidone, dimethylacetamide, triethylamine, and/or N,N-diisopropylethylamine.

Embodiment 66. The method according to embodiment 64, wherein the compound of Formula Int-1 is recrystallized prior to combining with the compound of Formula Int-2.

Embodiment 67. The method according to any one of embodiments 48 to 66, wherein the method results in an increased synthetic yield relative to a method for making the diacetate salt of Compound of Formula (I) which does not comprise slurrying the Compound of Formula (I) and/or isolating the diacetate salt of Compound of Formula (I).

Embodiment 68. The method according to any one of embodiments 48 to 66, wherein the method results in fewer process impurities in a composition comprising the diacetate salt of Compound of Formula (I) relative to a method for making the diacetate salt of Compound of Formula (I) which does not comprise slurrying the Compound of Formula (I) and/or isolating the diacetate salt of Compound of Formula (I).

Embodiment 69. The method according to embodiment 68, wherein the method results in a decrease in the concentration of process impurities.

Embodiment 70. The method according to embodiment 68, wherein the method results in a concentration of less than 1% of process impurities, as determined by LC.

Embodiment 71. The method according to embodiment 68, wherein the method results in a lower number of process impurities.

Embodiment 72. The method according to embodiment 68, wherein the method results in three fewer process impurities.

Embodiment 73. A diacetate salt of Compound of Formula (I) obtained by the method according to any one of embodiments 48 to 72.

Embodiment 74. A pharmaceutical composition comprising a diacetate salt of Compound of Formula (I) formed from combining the diacetate salt of Compound of Formula (I) produced according to the method of any one of embodiments 48 to 72 or the diacetate salt of Compound of Formula (I) according to embodiment 73 and at least one additional component chosen from pharmaceutically acceptable carriers, pharmaceutically acceptable vehicles, and pharmaceutically acceptable excipients.

Embodiment 75. A pharmaceutical composition consisting of a diacetate salt of Compound of Formula (I) formed from combining the diacetate salt of Compound of Formula (I) produced according to the method of any one of embodiments 48 to 72 or the diacetate salt of Compound of Formula (I) according to embodiment 73 and at least one additional component chosen from pharmaceutically acceptable carriers, pharmaceutically acceptable vehicles, and pharmaceutically acceptable excipients.

Embodiment 76. A pharmaceutical composition consisting essentially of a diacetate salt of Compound of Formula (I) formed from combining the diacetate salt of Compound of Formula (I) produced according to the method of any one of embodiments 48 to 72 or the diacetate salt of Compound of Formula (I) according to embodiment 73 and at least one additional component chosen from pharmaceutically acceptable carriers, pharmaceutically acceptable vehicles, and pharmaceutically acceptable excipients.

Embodiment 77. Form 2 of Compound of Formula (I).

Embodiment 78. Substantially pure Form 2 of Compound of Formula (I) according to embodiment 77.

Embodiment 79. Form 2 of Compound of Formula (I) according to embodiment 77 or 78, wherein the Form 2 is at least 98% pure, as determined by LC.

Embodiment 80. Form 2 of Compound of Formula (I) according to any one of embodiments 77 to 79 characterized by an x-ray powder diffractogram substantially as shown in FIG. 54.

Embodiment 81. Form 2 of Compound of Formula (I) according to any one of embodiments 77 to 80, characterized by an x-ray powder diffractogram having a signal at at least three two-theta values, ±0.2, chosen from 5.6, 7.6, 10.2, 10.6, 12.4, 15.1, 17.0, and 17.7.

Embodiment 82. Form 2 of Compound of Formula (I) according to any one of embodiments 77 to 81, characterized by an x-ray powder diffractogram having a signal at at least five two-theta values, ±0.2, chosen from 5.6, 7.6, 10.2, 10.6, 12.4, 15.1, 17.0, and 17.7.

Embodiments 83. Form 2 of Compound of Formula (I) according to any one of embodiments 77 to 82, characterized by an x-ray powder diffractogram having a signal at at least seven two-theta values, ±0.2, chosen from 5.6, 7.6, 10.2, 10.6, 12.4, 15.1, 17.0, and 17.7.

Embodiment 84. A method for making Form 2 of Compound of Formula (I) comprising isolating Form 2 from a mixture comprising Compound of Formula (I) and at least one first solvent.

Embodiment 85. The method according to embodiment 84, wherein the mixture further comprises at least one second solvent.

Embodiment 86. The method according to embodiment 84 or 85, wherein the at least one first solvent is acetone.

Embodiment 87. The method according to any one of embodiments 84 to 86, wherein the at least one second solvent is water.

Embodiment 88. The method according to any one of embodiments 85 to 87, wherein the ratio of the volume of the at least one first solvent to the volume of the at least one second solvent ranges from 1/1 to 99/1.

Embodiment 89. The method according to any one of embodiments 85 to 88, wherein the ratio of the volume of the at least one first solvent to the volume of the at least one second solvent is 95/5.

Embodiment 90. A method for making Form 2 of Compound of Formula (I) comprising:

slurrying Compound of Formula (I) in a solvent mixture comprising acetone and water.

Embodiment 91. The method according to embodiment 90, further comprising:

combining a compound of Formula Int-3:

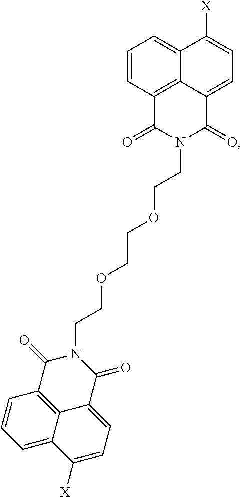

wherein X is chosen from F, Cl, Br, and I, and a compound of Formula Int-2:

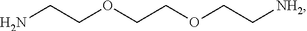

to form the Compound of Formula (I).

Embodiment 92. The method according to embodiment 91, further comprising:

combining a compound of Formula Int-1:

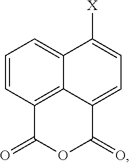

wherein X is chosen from F, Cl, Br, and I, and the compound of Formula Int-2 to form the compound of Formula Int-3.

Embodiment 93. The method according to any one of embodiments 90 to 92, wherein the ratio of the volume of acetone to the volume of water ranges from 99/1 to 1/99.

Embodiment 94. The method according to embodiment 93, wherein the ratio of the volume of acetone to the volume of water is 95/5.

Embodiment 95. Form 2 of Compound of Formula (I) obtained by the method according to any one of embodiments 84 to 94.

Embodiment 96. A pharmaceutical composition comprising Compound of Formula (I) formed from combining Form 2 of the Compound of Formula (I) according to any one of embodiments 77 to 83 or 95 and at least one additional component chosen from pharmaceutically acceptable carriers, pharmaceutically acceptable vehicles, and pharmaceutically acceptable excipients.

Embodiment 97. A pharmaceutical composition consisting of Compound of Formula (I) formed from combining Form 2 of the Compound of Formula (I) according to any one of embodiments 77 to 83 or 95 and at least one additional component chosen from pharmaceutically acceptable carriers, pharmaceutically acceptable vehicles, and pharmaceutically acceptable excipients.

Embodiment 98. A pharmaceutical composition consisting essentially of Compound of Formula (I) formed from combining Form 2 of the Compound of Formula (I) according to any one of embodiments 77 to 83 or 95 and at least one additional component chosen from pharmaceutically acceptable carriers, pharmaceutically acceptable vehicles, and pharmaceutically acceptable excipients.

In order that the disclosure herein may be more fully understood, the following examples are set forth. It should be understood that these examples are for illustrative purposes only and are not to be construed as limiting this disclosure in any manner.

### EXAMPLES

Abbreviations

The following abbreviations are used herein:
ACN: acetonitrile
DCM: dichloromethane
DMF: N,N-dimethylformamide
DMSO: dimethylsulfoxide
DSC: differential scanning calorimetry
DVS: dynamic vapor sorption
EtOH: ethanol
EtOAc: ethyl acetate
Et₂O: diethyl ether
HFIPA: 1,1,1,3,3,3-hexafluoro-2-propanol
HPLC: high performance liquid chromatography
MeOH: methanol
MEK: methyl ethyl ketone
2-MeTHF: 2-methyltetrahydrofuran
NMR: nuclear magnetic resonance
2-PrOH: 2-propanol
TG: thermogravimetric
THF: tetrahydrofuran
TFE: 2,2,2-trifluoroethanol
UPLC: ultra performance liquid chromatography
XRPD: x-ray powder diffraction

General methods and experimental details for preparing Compound of Formula (I) of the present disclosure are set forth below.

X-Ray Powder Diffraction

The Rigaku Smart-Lab X-ray diffraction system was configured for reflection Bragg-Brentano geometry using a line source X-ray beam. The x-ray source was a Cu Long Fine Focus tube that was operated at 40 kV and 44 ma. That source provided an incident beam profile (at the sample) that changed from a narrow line at high angles to a broad rectangle at low angles. Beam conditioning slits were used on the X-ray source to ensure that the maximum beam size is less than 10 mm, both along the line and normal to the line. The Rigaku Smart-Lab was operated to give peak widths of 0.1 degrees two-theta or less. The axial divergence of the X-ray beam was controlled by 5.0-degree Soller slits in both the incident and diffracted beam paths.

Powder samples were prepared in a low-background Si holder using light manual pressure to keep the sample surfaces flat and level with the reference surface of the sample holder. Each sample was analyzed from 2 to 40 degrees two-theta using a continuous scan of 6 degrees two-theta per minute, with an effective step size of 0.02 degrees two-theta.

Differential Scanning Calorimetry

DSC analyses were carried out employing a TA Instruments 02000 instrument. The instrument temperature calibration was performed using an indium sample. The DSC cell was kept under a nitrogen purge of ˜50 mL/minute during each analysis. The sample was placed in a standard, crimped, aluminum pan and was heated from 25° C. to 350° C., at a rate of 10° C./minute.

Thermogravimetric Analysis

Thermogravimetric analysis was carried out using a TA Instruments Q50 instrument. The instrument balance was calibrated using class M weights and the temperature calibration was performed using alumel. The nitrogen purge was ˜40 mL/minute at the balance and ˜60 mL/minute at the furnace. Each sample was placed into a pre-tared platinum pan and heated from 20° C. to 350° C. at a rate of 10° C./minute.

Dynamic Vapor Sorption Analysis

DVS analysis was carried out using a TA Instruments Q5000 Dynamic Vapor Sorption analyzer. The instrument was calibrated with standard weights and a sodium bromide standard for humidity. Approximately 10-25 mg of sample was loaded into a metal-coated quartz pan for analysis. The sample was analyzed at 25° C. with a maximum equilibration time of one hour in 10% relative humidity (RH) steps from 5% to 95% RH (adsorption cycle) and from 95% to 5% RH (desorption cycle). The movement from one step to the next occurred either after satisfying the equilibrium criterion of 0.01% weight change or, if the equilibrium criterion was not met, after one hour. The percent weight change values were calculated using Microsoft Excel®.

Optical Microscopy

Optical microscopy experiments were carried out employing a Leica DM 2500 P compound microscope. The sample was placed onto a glass slide and images were captured using a QImaging MicroPublisher 3.3 RTV camera. Images were collected at 10× magnification.

Nuclear Magnetic Resonance Spectroscopy

Solution-phase proton NMR (¹H-NMR) spectra were acquired on a Bruker DRX-500 spectrometer. Samples were prepared by dissolving material in DMSO-d₆, filtering, and placing the samples into individual 5-mm NMR tubes for subsequent spectral acquisition. The temperature was maintained at 298 K. A 5-mm cryoprobe operating at an observing frequency of 499.89 MHz was used.

Ultra Performance Liquid Chromatography

Purity of Compound of Formula (I) or a diacetate salt of Compound of Formula (I) was determined using ultra performance liquid chromatography. Specifications for the instrument and column are shown in Table 1.

TABLE 1
Ultra performance liquid chromatography conditions

| Instrument | Agilent 1290 Infinity II UPLC | | |
|---|---|---|---|
| Column | Waters Acquity UPLC BEH C18, 2.1 × | | |
| | 100 mm, 1.7 μm | | |
| Gradient | Time | % A | % B |
| | 0 | 95 | 5 |
| | 0.3 | 95 | 5 |
| | 2.0 | 0 | 100 |
| | 5.5 | 0 | 100 |
| | 6.0 | 95 | 5 |
| | 7.0 | 95 | 5 |

| | |
|---|---|
| Flow Rate | 0.61 mL/min |
| Injection Volume | 1.00 μL |
| Wait Time After Draw: | 0.8 s |
| Injection Valve to Bypass | Checked |
| for Delay Volume | |
| Reduction: | |
| Wavelength | 210 nm and 440 nm |
| Column Temp | 30° C. |
| Run Time | 7 minutes |

Mobile phase A is a 0.1% (v:v) aqueous solution of trifluoroacetic acid. Mobile phase B is a mixture of 90% by volume acetonitrile and 10% by volume mobile phase A. Purity of the Compound of Formula (I) and amounts of impurities were calculated using the following formulae:

a. Purity
The percent of Compound of Formula (I) in each sample was calculated using the following formula:

$$Assay ( \% ) = \frac{A s}{A w} \times \frac{C w}{C s} \times 100 \%$$

wherein,
As=Peak area of sample tested
Aw=Average peak area of all injections of the working standard
Cw=Concentration of working standard (in μg/mL)
Cs=Concentration of sample tested (in μg/mL)

b. Impurities
The percent of each impurity in each sample was calculated using the following formula:

$$Impurity ( \% ) = \frac{A s}{A w} \times \frac{C w}{C s} \times 100 \%$$

wherein,
As=Peak area of impurity in sample tested
Aw=Average peak area of all injections of a working standard
Cw=Concentration of a working standard (in μg/mL)
Cs=Concentration of sample tested (in μg/mL)

### Example 1: Polymorph Screening

Compound of Formula (I) was exposed to a variety of solvents and conditions, as shown in Table 1, to detect and determine any polymorphic properties of Compound of Formula (I). Samples obtained from each experiment were analyzed by XRPD and the results and corresponding figures are listed in the right-hand column of Table 2.

TABLE 2
Polymorph screening conditions.

| Method | Solvent | Conditions | Solid Form | FIG. |
|---|---|---|---|---|
| Cooling | HFIPA | acetone (AS), 60° C. → −15° C. | non-crystalline | 1 |
| | | ACN (AS), 60° C. → −15° C. | Form 1 | 2 |
| | | dioxane (AS), 60° C. → −15° C. | non-crystalline | 3 |
| | | EtOH (AS), 60° C. → −15° C. | Form 1 | 4 |
| | | EtOAc (AS), 60° C. → −15° C. | non-crystalline | 5 |
| | | MeOH (AS), 60° C. → −15° C. | Form 1 | 6 |
| | | MEK (AS), 60° C. → −15° C. | Form 1 | 7 |
| | | 2-MeTHF (AS), 60° C → −15° C. | non-crystalline | 8 |
| | | 2-PrOH (AS), 60° C. → −15° C. | non-crystalline | 9 |
| | | THF (AS), 60° C. → −15° C. | non-crystalline | 10 |
| | | toluene (AS), 60° C. → −15° C. | Form 1 | 11 |
| | | water (AS), 60° C. → −15° C. | non-crystalline | 12 |
| | TFE | acetone (AS), 60° C. → −15° C. | Form 1 | 13 |
| | | ACN (AS), 60° C. → −15° C. | Form 1 | 14 |
| | | dioxane (AS), 60° C. → −15° C. | non-crystalline | 15 |
| | | EtOH (AS), 60° C. → −15° C. | Form 1 | 16 |
| | | EtOAc (AS), 60° C. → −15° C. | Form 1 | 17 |
| | | MeOH (AS), 60° C. → −15° C. | Form 1 | 18 |
| | | MEK (AS), 60° C. → −15° C. | Form 1 | 19 |
| | | 2-MeTHF (AS), 60° C. → −15° C. | Form 1 | 20 |
| | | 2-PrOH (AS), 60° C. → −15° C. | Form 1 | 21 |
| | | THF (AS), 60° C. → −15° C. | Form 1 | 22 |
| | | toluene (AS), 60° C. → −15° C. | Form 1 | 23 |
| | | water (AS), 60° C. → −15° C. | non-crystalline | 24 |
| Evaporation | HFIPA | open vial, room temp. | Form 1 | 25 |
| | TFE | open vial, room temp. | Form 1 | 26 |
| Slurry | acetone | Room temp., 7 days | non-crystalline | 27 |
| | | 55° C., 7 days | Form 1 | 28 |
| | ACN | Room temp., 7 days | Form 1 | 29 |
| | | 55° C., 7 days | Form 1 | 30 |
| | chloroform | Room temp., 7 days | Form 1 | 31 |
| | DCM | Room temp., 7 days | Form 1 | 32 |
| | 1,4-dioxane | Room temp., 7 days | Form 1 | 33 |
| | DMF | Room temp., 7 days | Form 1 | 34 |
| | | 55° C., 7 days | Form 1 | 35 |
| | DMSO | Room temp., 7 days | Form 1 | 36 |
| | EtOH | Room temp., 7 days | Form 1 | 37 |
| | | 55° C., 7 days | Form 1 | 38 |
| | EtOAc | Room temp., 7 days | Form 1 | 39 |
| | | 55° C., 7 days | Form 1 | 40 |
| | Et₂O | Room temp., 7 days | Form 1 | 41 |
| | | 40° C., 7 days | Form 1 | 42 |
| | MeOH | Room temp., 7 days | Form 1 | 43 |
| | | 55° C., 7 days | Form 1 | 44 |
| | MEK | Room temp., 7 days | Form 1 | 45 |
| | | 55° C., 7 days | Form 1 | 46 |
| | 2-MeTHF | Room temp., 7 days | Form 1 | 47 |
| | 2-PrOH | Room temp., 7 days | Form 1 | 48 |
| | THF | Room temp., 7 days | non-crystalline | 49 |
| | | 55° C., 7 days | Form 1 | 50 |
| | toluene | Room temp., 7 days | Form 1 | 51 |
| | water | Room temp., 7 days | Form 1 | 52 |
| | | 55° C., 7 days | Form 1 | 53 |
| | acetone/water | Room temp., 7 days | Form 2 | 54 |
| | (95/5, v/v) | | Form 2 | 55 |
| | CAN/water | Room temp., 7 days | Form 1 | 56 |
| | (95/5, v/v) | | | |
| | EtOH/water | Room temp., 7 days | Form 1 | 57 |
| | (95/5, v/v) | | | |
| | THF/water | Room temp., 7 days | Form 1 | 58 |
| Heating | none | 145° C., 1 min. | Form 1 | 59 |

AS = anti-solvent

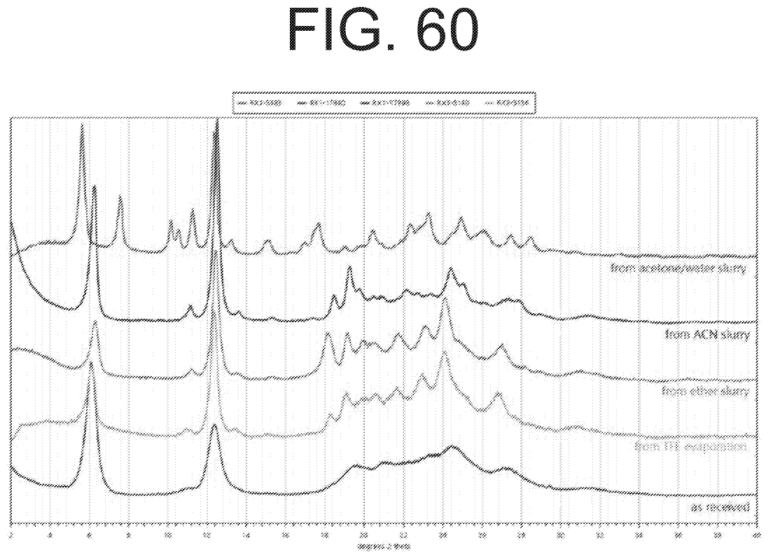

Compound of Formula (I) was surprisingly and unexpectedly found to exist in three solid forms: Form 1 (low-crystalline), Form 2 (crystalline), and non-crystalline. FIG. 60 shows an overlay comparison of XRPD patterns of samples of Compound of Formula (I) obtained from: an acetone/water slurry (trace A; Form 2); an acetonitrile slurry (trace B; Form 1); ether slurry (trace C; Form 1); 2,2,2-trifluoroethanol evaporation (trace D; Form 1); and synthetic method similar to the method of Example 4 (trace E; Form 1).

### Example 2: Characterization of Solid Forms

As discussed in Example 1, it was surprisingly and unexpectedly discovered that Compound of Formula (I) can exist in three solid state forms: Form 1 (low-crystalline), Form 2 (crystalline), and non-crystalline. Form 1 and Form 2 were characterized according to the analytical methods described herein.

Form 1 of Compound of Formula (I)

Form 1 was obtained from stirring a slurry of acetonitrile and Compound of Formula (I) at 55° C. for 7 days.

X-Ray Powder Diffraction

The product was analyzed using x-ray powder diffraction. FIG. 30 shows an XRPD pattern for this sample. Based on the XRPD pattern, this sample was determined to be Form 1. A list of positions and relative intensities of signals in the XRP diffractogram for this sample of Form 1 is below in Table 3.

TABLE 3
XRPD Signals (degrees two-theta) displayed
by Form 1 of Compound of Formula (I).

| Position | Relative Intensity |
|---|---|
| (degrees two-theta) | (%) |
| 6.2 | 71.22 |
| 11.1 | 4.33 |
| 12.5 | 100 |
| 13.6 | 3.41 |
| 15.3 | 1.92 |
| 18.5 | 10.19 |
| 19.3 | 19.58 |
| 19.8 | 6.51 |
| 20.9 | 3.5 |
| 22.1 | 4.4 |
| 24.4 | 13.74 |
| 25 | 6.75 |
| 27.2 | 3.78 |
| 27.8 | 5.24 |
| 31.2 | 2.37 |

Differential Scanning Calorimetry

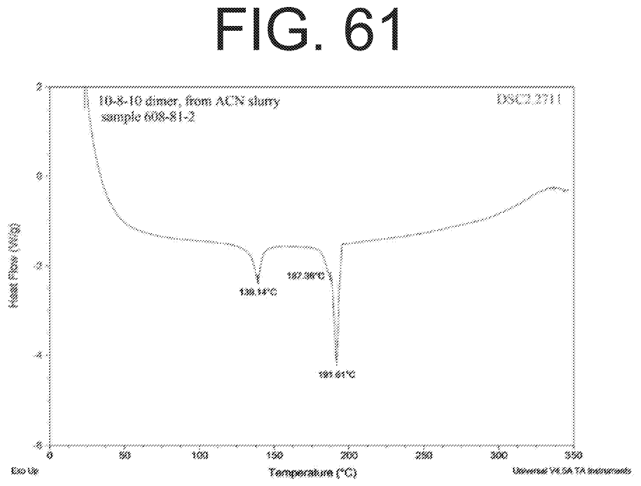

Form 1 was subjected to differential scanning calorimetry. FIG. 61 shows a DSC trace for this Form. Endotherms were observed at 139.1° C. and 191.6° C., along with a shoulder at 187.4° C.

Dynamic Vapor Sorption

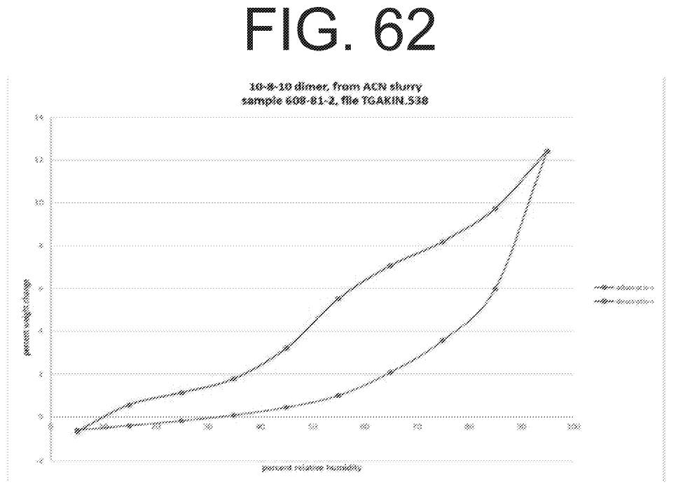

Form 1 was analyzed by dynamic vapor sorption. FIG. 62 shows a DVS trace for this sample of Form 1. The following weight changes were observed: 0.6% weight loss upon drying at 5% relative humidity; 13.04% weight gain when cycling from 5% to 95% relative humidity; and 13.12% weight loss when cycling from 95% to 5% relative humidity.

Post-DVS X-Ray Powder Diffraction Analysis

After Form 1 was subjected to DVS cycling, it was analyzed using x-ray powder diffraction. FIG. 63 shows an XRPD pattern for this sample. It was found to be unchanged relative to XRPD analysis of the same sample prior to DVS analysis (see FIG. 30).

¹H-Nuclear Magnetic Resonance Spectroscopy

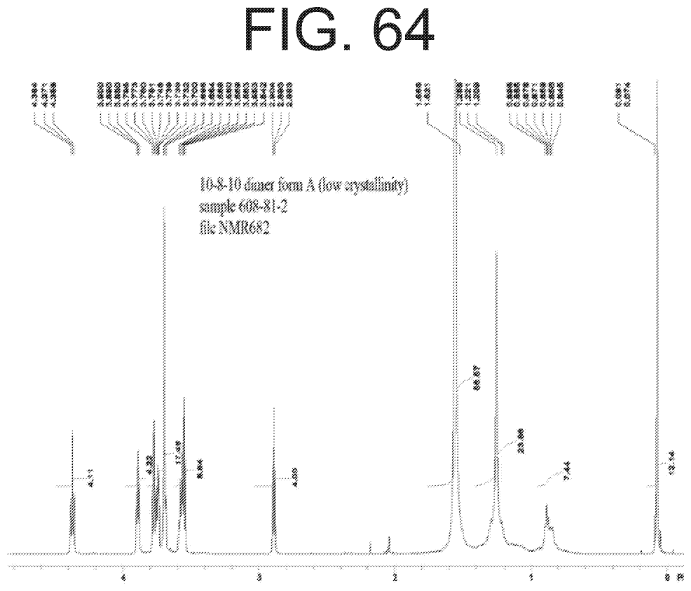

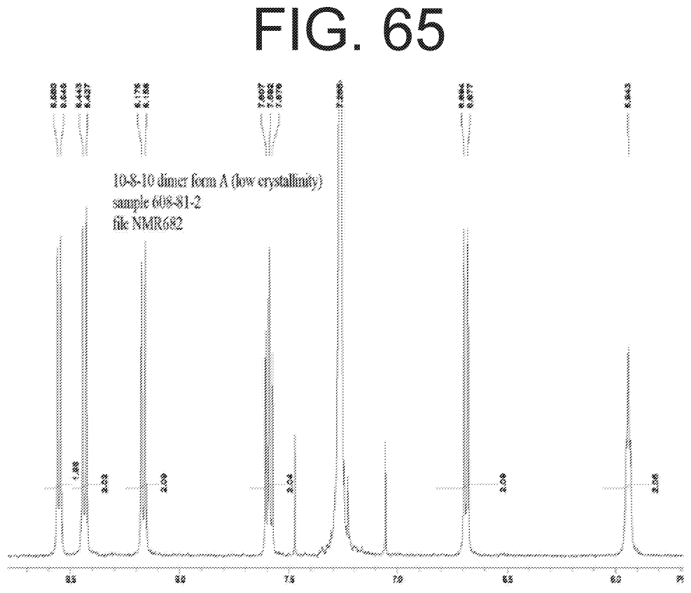

Form 1 was analyzed by ¹H-NMR spectroscopy. FIGS. 64 and 65 show the obtained spectra. The ¹H-NMR spectra were consistent with the structure of Compound of Formula (I) (signals from some impurities below 2.5 ppm were present).

Form 2 of Compound of Formula (I)

Form 2 of Compound of Formula (I) was obtained from stirring a slurry of acetone/water (95/5, v/v) and Compound of Formula (I) at room temperature for 7 days.

X-Ray Powder Diffraction

The product was analyzed using x-ray powder diffraction. FIG. 54 shows an XRPD pattern for this sample. Based on the XRPD pattern, this sample was determined to be Form 2. A list of positions and relative intensities of signals in the XRP diffractogram for this sample of Form 2 is below in Table 4.

TABLE 4
XRPD Signals (degrees two-theta) displayed
by Form 2 of Compound of Formula (I).

| Position | Relative Intensity |
|---|---|
| (degrees two-theta) | (%) |
| 5.6 | 88.4 |
| 7.6 | 34.1 |
| 10.2 | 20 |
| 10.6 | 14.4 |
| 11.3 | 33.13 |
| 12.4 | 100 |
| 13.2 | 7.8 |
| 15.1 | 11.5 |
| 17.0 | 6.7 |
| 17.7 | 37.2 |
| 19.0 | 2 |
| 19.8 | 2.3 |
| 20.4 | 13.6 |
| 22.3 | 20.4 |
| 23.3 | 23.1 |
| 24.9 | 21.5 |
| 26.1 | 17 |
| 27.4 | 7.7 |
| 28.4 | 9 |

Differential Scanning Calorimetry

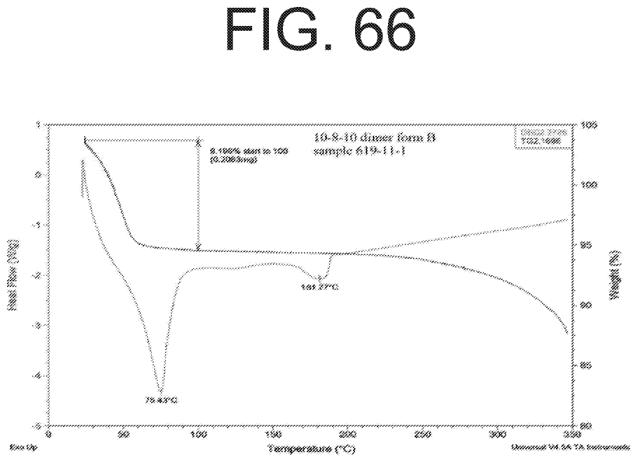

Form 2 was analyzed by differential scanning calorimetry. FIG. 66 shows a DSC trace for this sample. Endotherms were observed at 75.4° C. and 181.3° C.

Thermogravimetric Analysis

Form 2 was analyzed by thermogravimetric analysis. FIG. 66 shows a TGA trace for this sample. A weight loss of 9.2% was observed when heating from room temperature to 100° C., which may indicate the loss of 4.5 moles of water or 1.4 moles of acetone.

Dynamic Vapor Sorption

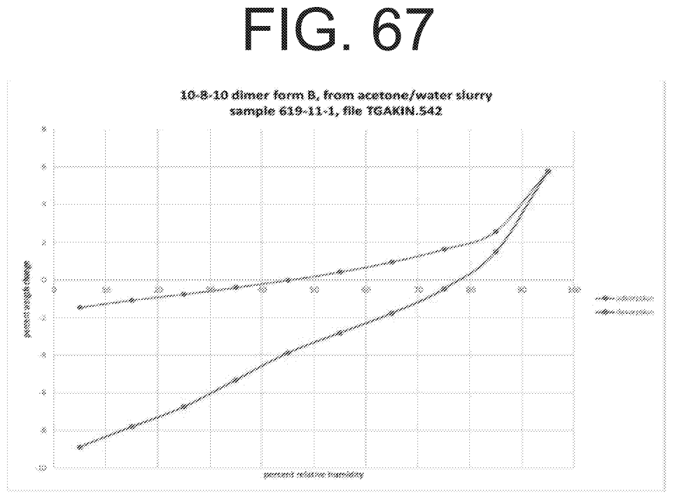

Form 2 was analyzed by dynamic vapor sorption. FIG. 67 shows a DVS trace for this sample. The following weight changes were observed: 1.44% weight loss upon drying at 5% relative humidity; 7.2% weight gain when cycling from 5% to 95% relative humidity; and 14.63% weight loss when cycling from 95% to 5% relative humidity.

Post-DVS X-Ray Powder Diffraction Analysis

After the sample of Form 2 was subjected to DVS cycling, it was analyzed using x-ray powder diffraction. FIG. 68 shows an XRPD pattern for this sample. The sample was found to be less crystalline relative to XRPD analysis of the same sample prior to DVS analysis (see FIG. 54).

Post-Heating X-Ray Powder Diffraction Analysis

A sample of Form 2 was heated at 80° C. for 20 minutes. After cooling, the sample was analyzed using x-ray powder diffraction. FIG. 69 shows an XRPD pattern for this sample. The sample was found to be less crystalline than the same sample prior to DVS analysis (see FIG. 54).

¹H-Nuclear Magnetic Resonance Spectroscopy

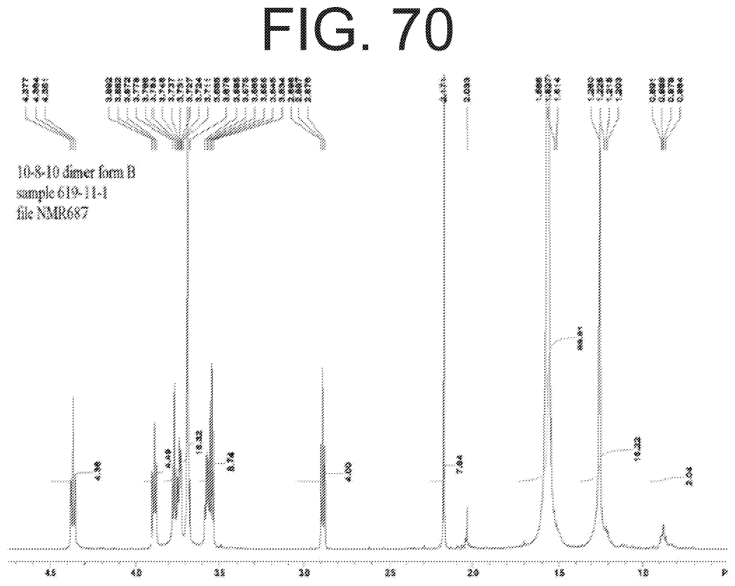

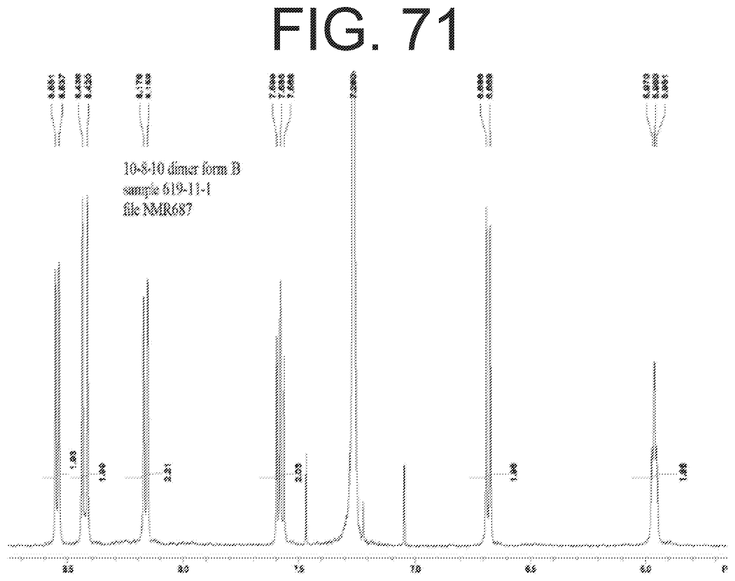

Form 2 was analyzed by ¹H-NMR spectroscopy. FIGS. 70 and 71 show the obtained spectra. The ¹H-NMR spectra are consistent with the structure of Compound of Formula (I) (some signals from impurities are present below 2.5 ppm). The ¹H-NMR spectra also show the presence of 1.3 moles of acetone, suggesting that Form 2 is an acetone solvate.

### Example 3: Characterization of Compound of Formula (I)

The isolated sample of Compound of Formula (I), synthesized by a method similar to the method of Example 4, was analyzed by methods described herein.

X-Ray Powder Diffraction

Compound of Formula (I) was analyzed using x-ray powder diffraction. FIG. 72 shows an XRPD pattern for this sample. Based on the XRPD pattern, this sample was determined to be Form 1. A peak listing is shown below in Table 5.

TABLE 5
XRPD Signals (degrees two-theta) displayed
by sample of Compound of Formula (I).

| Position | Relative Intensity |
|---|---|
| (degrees two-theta) | (%) |
| 6.1 | 100 |
| 11.3 | 8.3 |
| 12.5 | 64.5 |
| 20 | 10.6 |
| 23.3 | 6.4 |
| 24.7 | 17.2 |

Differential Scanning Calorimetry

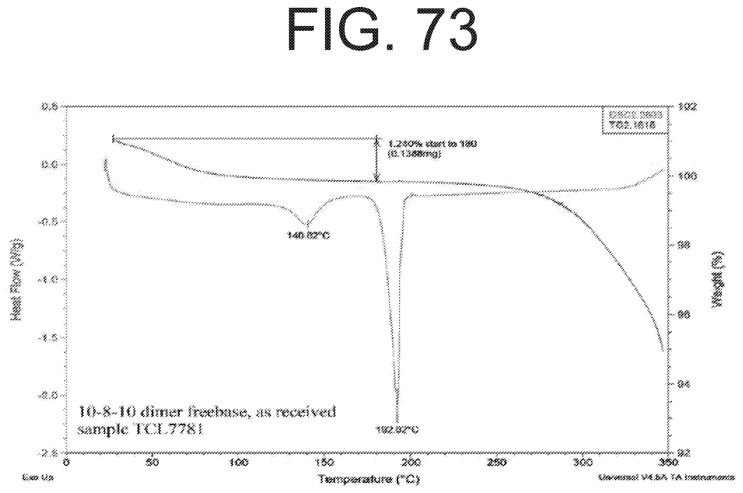

Compound of Formula (I) was subjected to differential scanning calorimetry. FIG. 73 shows a DSC trace for this sample. Endotherms were observed at 140.8° C. and 192.0° C.

Thermogravimetric Analysis

Compound of Formula (I) was subjected to thermogravimetric analysis. FIG. 73 shows a TGA trace for this sample. A weight loss of 1.2% was observed when heating from room temperature to 180° C.

Dynamic Vapor Sorption

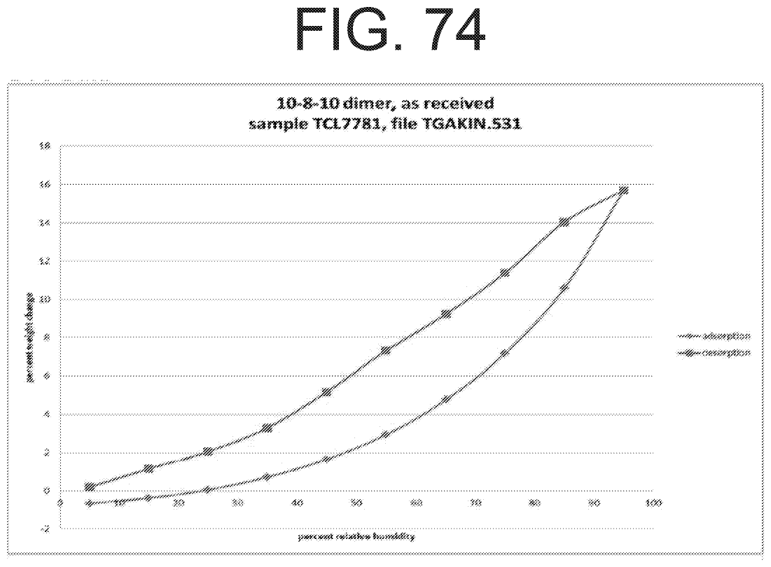

Compound of Formula (I) was subjected to dynamic vapor sorption analysis. FIG. 74 shows a DVS trace for this sample. The following weight changes were observed: 0.66% weight loss upon drying at 5% relative humidity; 16.36% weight gain when cycling from 5% to 95% relative humidity; and 15.51% weight loss when cycling from 95% to 5% relative humidity.

Post-DVS X-Ray Powder Diffraction Analysis

After Compound of Formula (I) was subjected to DVS cycling, it was analyzed using x-ray powder diffraction. FIG. 75 shows an XRPD pattern for this sample. It was found to be unchanged relative to XRPD analysis of the same sample prior to DVS analysis (see FIG. 72).

Optical Microscopy

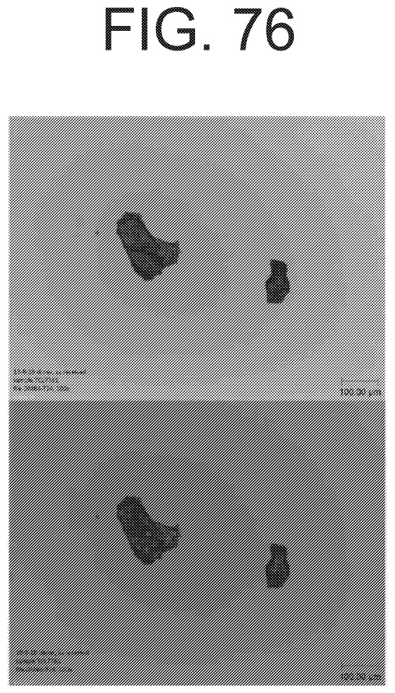

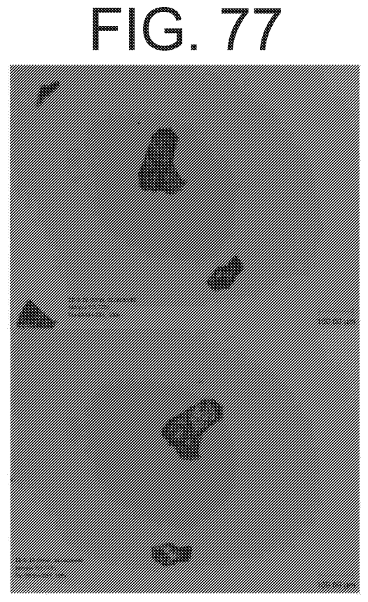

Compound of Formula (I) was examined using a microscope at 10× magnification. FIGS. 76A, 76B, 77A, and 77B show the isolated solid.

¹H-Nuclear Magnetic Resonance Spectroscopy

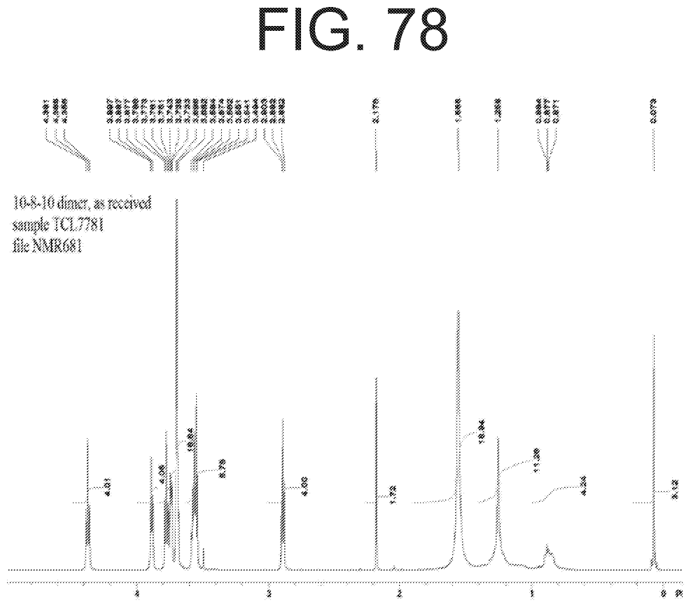

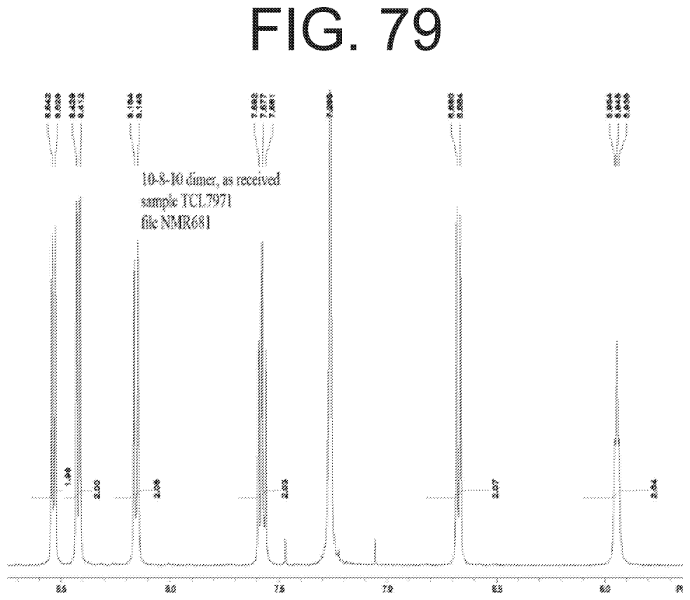

Compound of Formula (I) was analyzed by ¹H-NMR spectroscopy. FIGS. 78 and 79 show the obtained spectra. The ¹H-NMR spectra are consistent with the structure of Compound of Formula (I) (signals from some impurities appear below 2.5 ppm).

Solubility Measurements

The mass of a sample of Compound of Formula (I) was determined, and to that sample was added an aliquot of solvent, as shown in Table 5. Visual inspection was used to determine whether dissolution occurred. The solubility of Compound of Formula (I) in each solvent was calculated by dividing the weight of the sample by the total amount of solvent used to dissolve the sample. Table 6 lists the solubility of Compound of Formula (I) in each solvent.

TABLE 6
Solubility of Compound of Formula (I) in Various Solvents.

| | Sample | Solvent | |
|---|---|---|---|
| | Weight | Amount | Solubility |
| Solvent | (mg) | (mL) | (mg/mL) |
| acetone | 15.3 | 15 | <1 |
| acetonitrile (CAN) | 17.3 | 15 | <1 |
| dichloromethane (DCM) | 18.2 | 15 | <1 |
| 1,4-dioxane | 17.0 | 15 | <1 |
| N,N-dinnethylformamide (DMF) | 17.1 | 15 | <1 |
| dimethylsulfoxide (DMSO) | 16.1 | 15 | <1 |
| ethanol (EtOH) | 17.0 | 15 | <1 |
| ethyl acetate (EtOAc) | 18.6 | 15 | <1 |
| diethyl ether (Et₂O) | 18.3 | 15 | <1 |
| 1,1,1,3,3,3-hexafluoro-2-propanol | 17.6 | 1 | 18 |
| (HFIPA) | | | |
| methanol (MeOH) | 16.9 | 15 | <1 |
| methyl ethyl ketone (MEK) | 18.1 | 15 | <1 |
| 2-methyl tetrahydrofuran (2-MeTHF) | 18.1 | 15 | <1 |
| 2-propanol (2-PrOH) | 15.6 | 15 | <1 |
| tetrahydrofuran (THF) | 15.9 | 15 | <1 |
| 2,2,2-trifluoroethanol (TFE) | 17.5 | 1 | 18 |
| toluene | 16.5 | 15 | <1 |
| water | 15.8 | 15 | <1 |
| acetone/water (95/5, v/v) | 18.4 | 15 | <1 |
| acetonitrile/water (95/5, v/v) | 16.4 | 15 | <1 |
| ethanol/water (95/5, v/v) | 19.0 | 15 | <1 |
| tetrahydrofuran/water (95/5, v/v) | 18.7 | 15 | <1 |

### Example 4: Synthesis of Compound of Formula (I)

Scheme 1, below, depicts a synthesis of the Compound of Formula (I).

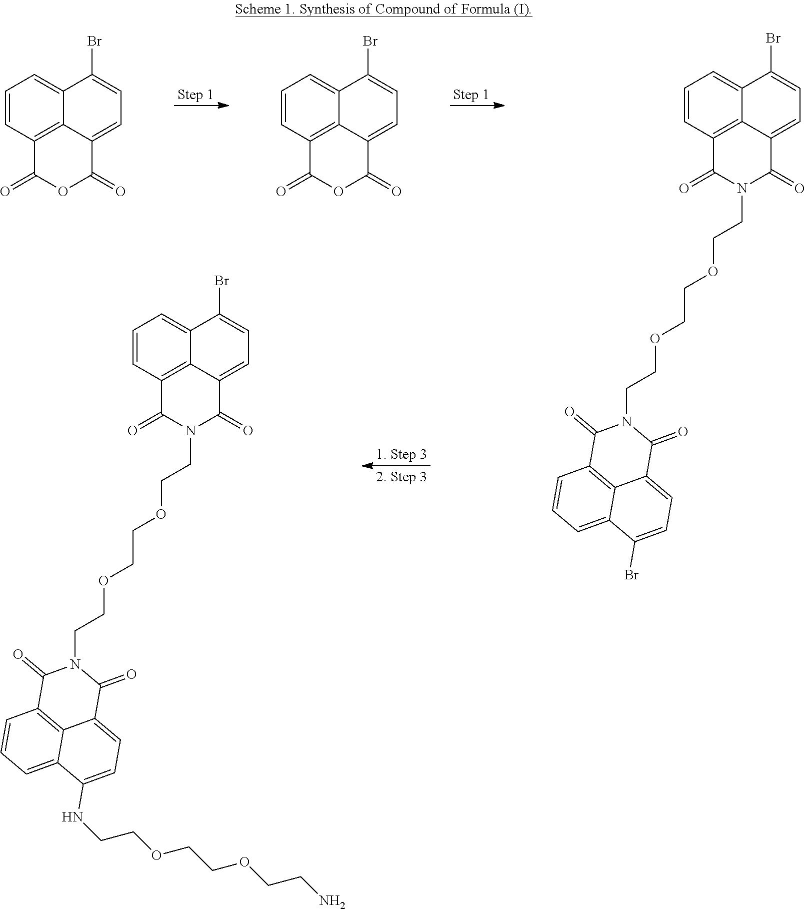 [SMILES: NCCOCCOCCNc1ccc2c3c(cccc13)C(=O)N(CCOCCOCCN1C(=O)c3cccc4c(Br)ccc(c34)C1=O)C2=O ; O=C1OC(=O)c2ccc(Br)c3cccc1c23 ; O=C1OC(=O)c2ccc(Br)c3cccc1c23 ; O=C1c2cccc3c(Br)ccc(c23)C(=O)N1CCOCCOCCN1C(=O)c2cccc3c(Br)ccc(c23)C1=O]

### Preparation of 4-bromo-1,8-naphthalic anhydride

Dimethylacetamide (5 volumes) and 4-bromo-1,8-naphthalic anhydride (1 equivalent) were combined and stirred while heating to 70° C. for approximately 1 hour. After heating, the mixture was allowed to cool to room temperature and stirred overnight. The resulting mixture was filtered and the filter cake was washed with dimethylacetamide (1.5 volumes), followed by tert-butyl methyl ether (4 volumes). The collected solid material was then dried to a constant weight in a vacuum oven at 65° C. to afford the title compound.

### Preparation of 2,2′-((ethane-1,2-diylbis(oxy))bis(ethane-2,1-diyl))bis(6-bromo-1H-benzo[de]isoquinoline-1,3(2H)-dione)

To a mixture of 4-bromo-1,8-naphthalic anhydride (2.5 equivalents) and dimethylacetamide (8 volumes) was added N,N-diisopropylethylamine (2.5 equivalents). The mixture was then cooled to 5±5° C. A mixture of 2,2′(ethylenedioxy)-bis(ethylamine) (1 equivalent) and dimethylacetamide (3 volumes) was then added to the previously cooled mixture of 4-bromo-1,8-naphthalic anhydride at a rate that maintained the temperature of the combined mixture no more than 25° C. After the addition was complete, the resulting mixture was stirred overnight (at least 12 hours) at room temperature. The mixture was then stirred at 80° C. for 6 hours. The resulting slurry was hot filtered, and the filter cake was washed with dimethylacetamide (4 washes at 3 volumes each) and then tert-butyl methyl ether (4 washes at 3 volumes each). The collected material was then dried to a constant weight in a vacuum oven at 45° C. to afford the title compound.

Preparation of Compound of Formula (I)

Step 3

A mixture of 2,2′-((ethane-1,2-diylbis(oxy))bis(ethane-2,1-diyl))bis(6-bromo-1H-benzo[de]isoquinoline-1,3(2H)-dione) (material used from Step 2), toluene (20 volumes) and 2,2′(ethylenedioxy)bis(ethylamine) (20 volumes) was stirred at 80° C. for at least 24 hours. Upon cooling, the mixture was stirred for at least 48 hours. The resulting slurry was then filtered and washed with toluene (3 washes at 7 volumes each) and then acetonitrile (4 washes at 7 volumes). The isolated cake was dried by being blown with a stream of nitrogen while on a filter and the material was shielded from ambient light. The acetonitrile content of the isolated material was determined via gas chromatography. The overall yield (i.e., steps 1-3) ranged from about 40.5% to about 44.5%. In contrast, other syntheses of a Compound of Formula (I) afford the desired material in much lower yield. See, e.g., U.S. Pat. No. 6,410,505 at cols. 9-10 (reporting yield of 23% achieved with a one-pot method).

Step 4

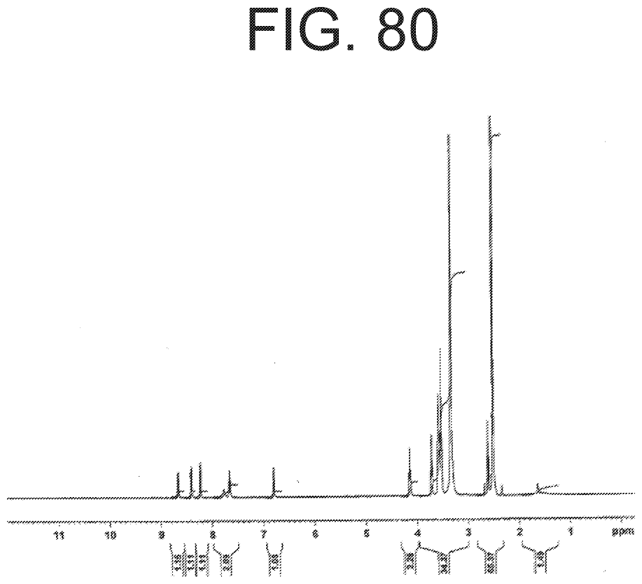

The product of Step 3 was combined with water (10 volumes) and acetonitrile (sufficient additional acetonitrile was added to result in a total of 10 volumes acetonitrile when combined with amount determined to be present in the product of Step 3). The mixture was then heated to reflux (approx. 80° C.), and maintained at that temperature for 15-20 minutes. Heating and agitation were ceased and the mixture was cooled to room temperature over 40 hours. The slurry was filtered under nitrogen and shielded from light. The filter cake was then washed 3 times with an acetonitrile/water mixture (3 volumes, 1/1). After washing and filtration, the yield of the material was determined to be about 59% at about 92.7% purity by UPLC. ESI m/z: 401.4 [M+H]⁺. FIG. 80 shows a ¹H-NMR spectrum of the isolated material.

### Example 5: Synthesis of Diacetate Salt of Compound of Formula (I)

A mixture of Compound of Formula (I) obtained from the method of Example 4 (1 equivalent), water (20 volumes), and acetic acid (2 equivalents) was stirred at room temperature for at least 5 hours. The resulting slurry was filtered through a celite pad and the celite pad was washed with water (about 0.5-1 volume). The purity of the filtrate was assessed using UPLC and determined to be about 90%.

The filtrate was transferred to another flask and to that flask was added activated carbon (0.1 mass equivalents relative to the amount of Compound of Formula (I) initially added). The resulting mixture was stirred at room temperature for at least 8 hours. After stirring finished, the purity of the mixture was assessed by UPLC, and then the mixture was filtered through a Celite® pad and then through a 0.2 μm membrane filter. The filter cake was washed with water (2 volumes).

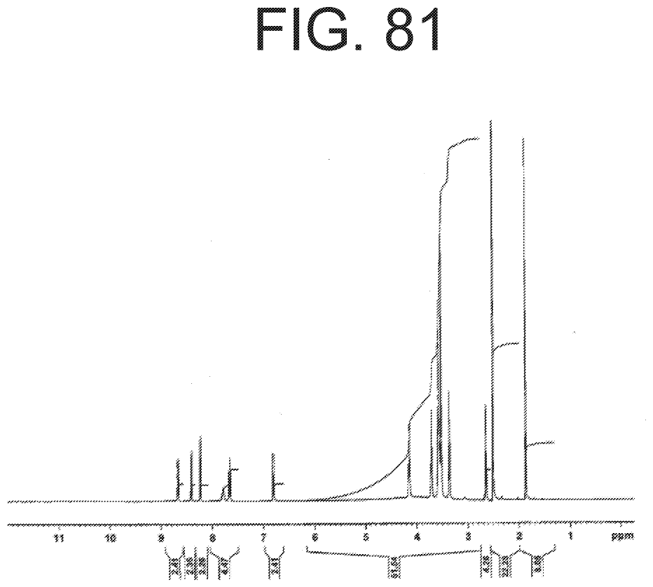

The resulting filtrate was then lyophilized to afford the title compound as a yellow/orange powder with a yield of about 70%. The purity was determined using UPLC to be greater than 98%. FIG. 81 shows a ¹H-NMR spectrum of the isolated material.

### Example 6: Purification of Diacetate Salt of Compound of Formula (I)

The diacetate salt of the Compound of Formula (I) produced according to Example 5 (prior to treatment with activated carbon) was subjected to filtration through various amounts of activated carbon to study the minimization and/or removal of process impurities. Table 5 sets out the various purification conditions and time points and shows the amount of various impurities that were identified after treatment of the diacetate salt of the Compound of Formula (I) with varying amounts of activated carbon. It was surprisingly found that treatment of the isolated material with increasing amounts of activated carbon led to a decrease in the total amount of impurity and, in some cases, elimination of some impurities. Purity was assessed using UPLC as described herein. The method of this Example resulted in a higher purity of the diacetate salt of the Compound of Formula (I) than the method of Example 7.

TABLE 7
Purity analysis after treatment with activated carbon. (N/D = not detected.)

| | | HPLC Area % | | | | | |
|---|---|---|---|---|---|---|---|
| | | Compound | | | | | |
| | Carbon | of Formula | Impurity | Impurity | Impurity | Impurity | Impurity |
| Time (day) | Treatment | (I) | #1 | #2 | #3 | #4 | #5 |
| Initial | None | 92.2 | 1.45 | 1.08 | 4.13 | 0.28 | 0.70 |
| 2 | #1 (500 mg) | 92.62 | 1.39 | 1.07 | 3.97 | 0.17 | 0.63 |
| 2 + 2 hours | #2 (1 g) | 93.34 | 1.25 | 0.91 | 3.72 | 0.16 | 0.47 |
| 2 + 4 hours | #2 (1 g) | 93.4 | 1.18 | 0.97 | 3.70 | 0.14 | 0.47 |
| 2 + 6 hours | #2 (1 g) | 93.51 | 1.20 | 0.90 | 3.69 | 0.12 | 0.45 |
| Day 3 AM | #2 (1 g) | 93.95 | 1.16 | 0.84 | 3.49 | N/D | 0.42 |
| Day 3 PM | #3 (1.5 g) | 94.31 | 1.01 | 0.89 | 3.37 | N/D | 0.30 |
| Day 4 AM | #4 (2.0 g) | 95.46 | 0.70 | 0.72 | 2.94 | N/D | 0.18 |
| Day 4 PM | #5 (2.5 g) | 96.17 | 0.49 | 0.64 | 2.56 | N/D | 0.13 |
| Day 5 AM | #6 (3.0 g) | 97.15 | 0.22 | 0.56 | 2.07 | N/D | N/D |
| Day 5 PM | #6 | 97.72 | 0.13 | 0.45 | 1.70 | N/D | N/D |
| Day 6 AM | #7 (3.5 g) | 98.26 | N/D | 0.38 | 1.36 | N/D | N/D |
| Day 6 PM | #8 (4 g) | 98.57 | N/D | 0.33 | 1.10 | N/D | N/D |
| Day 7 AM | #9 (4.5 g) | 98.81 | N/D | 0.27 | 0.91 | N/D | N/D |
| Day 8 AM | #10 (5 g) | 99.15 | N/D | 0.20 | 0.65 | N/D | N/D |
| Day 9 AM | #11 (5.5 g) | 99.27 | N/D | 0.20 | 0.53 | N/D | N/D |
| Day 9 PM | #12 (6 g) | 99.43 | N/D | 0.17 | 0.39 | N/D | N/D |
| Day 10 AM | #13 (6.5 g) | 99.54 | N/D | 0.14 | 0.32 | N/D | N/D |
| Day 10 AM | #14 (7.0 g) | 99.57 | N/D | 0.14 | 0.30 | N/D | N/D |
| Day 11 PM | #15 (7.5 g) | 99.60 | N/D | 0.14 | 0.27 | N/D | N/D |
| Day 12 AM | #16 (8.0 g) | 99.65 | N/D | 0.12 | 0.23 | N/D | N/D |

1. A method for making Form 1 of 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione) (“Compound of Formula (I)”) comprising isolating Form 1 from a mixture comprising Compound of Formula (I) and at least one solvent chosen from acetonitrile, chloroform, dichloromethane, 1,4-dioxane, dimethylformamide, dimethylsulfoxide, ethanol, ethyl acetate, diethyl ether, methanol, methylethylketone, 2-methyl-tetrahydrofuran, 2-propanol, tetrahydrofuran, toluene, and water.

2-4. (canceled)

5. The method according to claim 1, wherein the at least one solvent is acetonitrile and water.

6-8. (canceled)

9. A method for making a Compound of Formula (I) comprising:
slurrying Compound of Formula (I) in a solvent mixture comprising at least one first solvent chosen from acetonitrile, isopropanol, acetone, ethanol, and tetrahydrofuran; and water.

10. The method according to claim 9, wherein the at least one first solvent is acetonitrile.

11. The method according to claim 9, further comprising:
combining a compound of Formula Int-3: 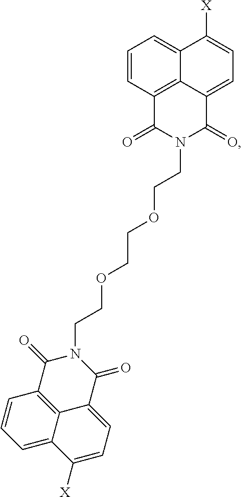
wherein X is chosen from F, Cl, Br, and I, and a compound of Formula Int-2: 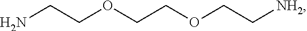
to form the Compound of Formula (I).

12. The method according to claim 11, wherein the combining of the compound of Formula Int-3 and the compound of Formula Int-2 is carried out in toluene.

13. The method according to claim 11, further comprising:
combining a compound of Formula Int-1: 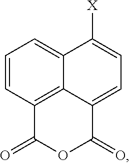
wherein X is chosen from F, Cl, Br, and I, and the compound of Formula Int-2 to form the compound of Formula Int-3.

14. The method according to claim 13, wherein the combining of the compound of Formula Int-1 and the compound of Formula Int-2 is carried out in dimethylformamide, dimethylsulfoxide, N-methyl-2-pyrrolidone, dimethylacetamide, triethylamine, and/or N,N-diisopropylethylamine.

15. The method according to claim 13, wherein the compound of Formula Int-1 is recrystallized prior to combining with a compound of Formula Int-2.

16. The method according to claim 10, wherein the ratio of acetonitrile to water is about 99/1 to about 1/99.

17. The method according to claim 10, wherein the ratio of acetonitrile to water is about 1/1.

18. The method according to claim 9, wherein the method results in an increased synthetic yield relative to a method for making Compound of Formula (I) which does not comprise slurrying Compound of Formula (I) in a solvent mixture comprising a first solvent and water.

19. The method according to claim 9, wherein the method results in fewer process impurities in a composition of Compound of Formula (I) relative to a method for making Compound of Formula (I) which does not comprise slurrying Compound of Formula (I) in a solvent mixture comprising a first solvent and water.

20. The method according to claim 19, wherein the method results in a decrease in the concentration of process impurities.

21. The method according to claim 19, wherein the method results in a concentration of less than 1% of process impurities, as determined by LC.

22. The method according to claim 19, wherein the method results in a lower number of process impurities.

23-47. (canceled)

48. A method for making a diacetate salt of Compound of Formula (I) comprising:
combining Compound of Formula (I) with at least one acid chosen from hydrochloric acid, hydrobromic acid, sulfuric acid, phosphoric acid, nitric acid, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, and polygalacturonic acid;
isolating the diacetate salt of Compound of Formula (I) from a mixture comprising the diacetate salt of Compound of Formula (I), activated carbon, and at least one solvent chosen from acetonitrile, ethanol, tetrahydrofuran, and water.

49-61. (canceled)

62. The method according to claim 48, further comprising:
combining a compound of Formula Int-3: 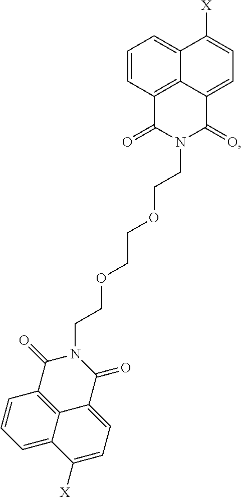
wherein X is chosen from F, Cl, Br, and I, and a compound of Formula Int-2: 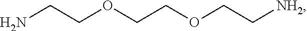
to form a Compound of Formula (I).

63-76. (canceled)

77. Form 2 of Compound of Formula (I).

78-80. (canceled)

81. Form 2 of Compound of Formula (I) according to claim 77, characterized by an x-ray powder diffractogram having a signal at least three two-theta values, ±0.2, chosen from 5.6, 7.6, 10.2, 10.6, 12.4, 15.1, 17.0, and 17.7.

82-98. (canceled)
